(19) United States
(12) Patent Application Publication
(10) Pub. No.: US 2009/0074666 A1
(43) Pub. Date: Mar. 19, 2009

(54) Peptide vaccine for influenza virus

(76) Inventors: Jonas Angstrom, Goteborg (SE); Halina Miller-Podraza, Vastra Frolunda (SE); Martina Pantzar, Saro (SE); Karl-Anders Karlsson, Goteborg (SE); Maria Blomqvist, Itasalmi (FI); Annamari Heiskanen, Helsinki (FI); Ritva Niemela, Helsinki (FI); Jari Helin, Vantaa (FI); Jari Natunen, Vantaa (FI); Tero Satomaa, Helsinki (FI); Olli Aitio, Helsinki (FI)

Correspondence Address: BIRCH STEWART KOLASCH & BIRCH, PO BOX 747, FALLS CHURCH, VA, 22040-0747, US

(21) Appl. No.: 11/918,791

(22) PCT Filed: Apr. 20, 2006
(86) PCT No.: PCT/FI2006/050157

§ 371 Date: Mar. 4, 2008

(30) Foreign Application Priority Data
Apr. 20, 2005 (FI) 200550405
Feb. 27, 2006 (FI) 20060200

(51) Int. Cl. A61K 39/145; G01N 33/566; A61B 10/00; G06G 7/58; A61P 31/16; C12Q 1/70; C07K 14/11

(52) U.S. Cl. 424/9.2; 436/501; 530/396; 424/186.1; 435/5; 703/11

(57) ABSTRACT

The invention relates to the method for evaluating the potential of a chemical entity, such as an antibody, to bind to a peptide epitope derived from the divalent sialoside binding site of hemagglutinin protein of influenza virus. The invention also provides peptide epitopes for use in the prevention and/or treatment of influenza or for the development of such treatment or vaccine against influenza.

### BACKGROUND OF THE INVENTION

Influenza virus infect the airways of a patient and initially cause general respiratory symptoms, which may result in high morbidity and mortality rates, especially in elderly persons. Thus, good targets for attacking the virus are constantly searched for. The significance of hemagglutinin protein of influenza virus in the pathogenesis of the virus has been known for a relatively long time. Consequently, in the field of vaccine and antibody development an aim has been to develop vaccines against conserved regions of influenza virus hemagglutinins. For example, a patent application of Takara Shuzo (EP0675199) describes antibodies which recognizes the stem region of certain influenza virus subtypes. WO0032228 describes vaccines containing hemagglutinin epitope peptides 91-108, 307-319, 306-324 and for non-caucasian populations peptide 458-467. Lu et al. 2002 describe a conserved site 92-105. Lin and Cannon 2002 describes conserved residues Y88, T126, H174, E181, L185 and G219. Hennecke et al. 2000 studied complex of hemagglutinin peptide HA306-318 with T-cell receptor and a HLA-molecule. Some conserved peptide structures have been reported in the primary binding site and a mutation which changes the binding specificity from α6-sialic acids to α3-sialic acids.

The present invention is also directed to a specific larger polylactosamine structure Neu5Acα6Galβ4GlcNAcβ3[Neu5Acα6Galβ4GlcNAcβ3Galβ4GlcNAcβ6]Galβ4Glc with specifically elongated β6-linked branch and analogues thereof, which bind to a specific large binding site on the surface of hemagglutinin. Preferred analogues include sialic acids such as natural or synthetic sialic acid analogues capable of replacing Neu5Ac in one or both of the sialic acid binding sites disclosed in the invention.

The present invention with large polylactosamines also abled the inventors to design analogs for the high affinity ligands for the large epitopes. The invention is specifically directed to epitopes according to the formula SAα3/6Galβ4[Glc(NAc)₀ₒᵣ₁)]₀ₒᵣ₁{β3Gal[β4Glc(NAc)₀ₒᵣ₁]₀ₒᵣ₁}₀ₒᵣ₁ linked with spacer to form dimeric or oligomeric or polymeric structures having specific distances between two active sialylated terminal structures, wherein SA is sialic acid, preferably N-acetylneuraminic acid, Neu5Ac. SA may be a natural or synthetic sialic acid analogue capable of replacing Neu5Ac in one or both of the sialic acid binding sites disclosed in the invention.

Previously, some random polymers of SAα6Galβ4Glc(NAc) and analogs has been represented including conjugates linked to polyacrylamide and other polymers (WO9814215). In another polymer based approach sialyllactoses were randomly conjugated on polyglutamic acid. The present invention is directed to conjugates having specific distances between two sialyl-epitopes.

Previously, trimeric conjugates of Neu5Acα3Galβ4Glc has been represented on cyclic peptides. The peptides were however designed to cross-link the traditional primary sialic acid binding epitopes on different domains of trimeric hemagglutinin protein and the distances between the epitopes are substantially longer than according the present invention (Organon of Japan, poster, International Glycoconjugate Meeting Haag, 2001).

The prior art further describes divalent sialic acid conjugates. These have moderately higher effect in blocking hemagglutination. It was assumed that the effect of the conjugates is based on the cross-linking two hemagglutinin surfaces on to each other, in face to face manner, while the present invention aims to cross-linking two sites on the same hemagglutinin. These prior art studies also described monosaccharide based dimers (Glick et al., 1991). From these studies it cannot be known if it is possible to cross-link larger oligosaccharides according to the invention and what kind of spacers would be needed to accomplish that.

Sialyloligosaccharide complexes with the primary sialic acid binding site of influenza hemagglutinin have been known for example with saccharide sequences Neu5Acα6Galβ4Glc, Neu5Acα6Galβ4GlcNAc and Neu5Acα6Galβ4GlcNAcβ3Galβ4Glc and similar a3-sialylated structures. A site close to the secondary site is known as complex with Neu5Acα3Galβ4Glc (Sauter et al., 1992). The orientation of the saccharide and the binding interactions and location of sialic acid are different in the secondary sialic acid binding site with the large polylactosamine epitopes according to the invention in comparison to the first report about the so called secondary binding site. No previous data seem to exist about saccharide binding to the bridging site which was revealed by the invention to be essential for the carbohydrate binding.

In a specific embodiment the present invention is further directed to polylactosamine epitopes with α3-sialylated polylactosamine epitopes. It was found out that branched α3-sialylated polylactosamine epitopes bind also effectively to some human influenza viruses. Branched structures were discovered to be clearly more effective and reproducible binders to influenza virus than corresponding non-branched structures with only one sialic acid. The binding strains includes avian type of viruses. It appears that the high affinity bindings caused by the polylactosamine backbone allow effective evolutionary changes between different types of terminally sialylated structures. Currently the influenza strains binding to human are more α6-sialic acid specific, but change may occur quickly. Therefore effective medicines against more “zoonotic” influenzas spreading to human from chicken or possibly from ducks need to be developed. There are examples of outbreaks of “chicken influenza” like the notorious Hong Kong −97 strain, which was luckily stopped by slaughtering all chickens in Hong Kong and thus resulted in only a few human casualties. The major fear of authorities such as WHO is the spread of such altered strains avoiding resistance in population based on the previous influenza seasons and leading to global infection, pandemy, of lethal viruses with probable α3-sialic acid binding. A major catastrophy of this type was the Spanish flu in 1918. An outbreak of an easily spreading influenza virus is very difficult to stop. There are currently effective medicines though sialidase inhibitors, if effective also against to non-human sialidases, could be of some use. In a preferred embodiment the present invention is directed to combined use of α3- and α6-sialylated polylactosamines against influenza viruses, especially human influenza viruses and in another embodiment against influenza viruses of cattle (/or wild animals) including especially pigs, horses, chickens(hens) and ducks.

The prior art describes binding to α3-sialylated polylactosamine structures including linear structures NeuNAcα3Galβ4GlcNAcβ3Galβ4GlcβCer and NeuNAcα3Galβ4GlcNAcβ3Galβ4GlcNAcβ3Galβ4GlcβCer and with similar binding effectivity NeuNAcα3Galβ4GlcNAcβ3(NeuNAcα3Galβ4GlcNAcβ6)Galβ4GlcNAcβ3Galβ4GlcβCer (Suzuki et al 1992). This article does not recognize the branched structure as highly activity possibly due to use of B Lee-strain which seem to have low selectivity among structures in contrast to many A strains used in the present invention. The present invention further shows the glycolipid independent activity of the structures.

In silico screening of ligands for a model structure is disclosed for instance in EP1118619 B1 and WO0181627.

### A BRIEF DESCRIPTION OF FIGURES AND SCHEMES

FIG. 1. Atomic coordinates of influenza virus hemagglutinin X-31 from PDB-database.

FIG. 2. The complex structure between influenza virus hemagglutinin and the oligosaccharide 7. Yellow structure indicates the oligosaccharide position. Some key aminoacid residues are marked with red.

FIG. 3. “Top view of the complex between the oligosaccharide 7 (yellow) and the influenza virus hemagglutinin, the upper structure. The red color indicate nonconserved aminoacids, white the N-glycan, and blue the conserved aminoacid in region close to the binding site. The structure below indicates the protein structure without the oligosaccharide.

FIG. 4. “Right side” view of the complex between the oligosaccharide 7 (yellow) and the influenza virus hemagglutinin, the upper structure. The red color indicate nonconserved aminoacids, white the N-glycan, and blue the conserved aminoacid in region close to the binding site. The structure below indicates the protein structure without the oligosaccharide.

FIG. 5. “Front view of the complex between the oligosaccharide 7 (yellow) and the influenza virus hemagglutinin, the upper structure. The red color indicate nonconserved aminoacids, white the N-glycan, and blue the conserved aminoacid in region close to the binding site. The structure on the right indicates the protein structure without the oligosaccharide.

FIG. 6. represents divalent conjugates of two Neu5Acα6LacNAc, structure 27, Table 3.

FIG. 7. represents divalent conjugates of two Neu5Acα3Lac, structure 26, Table 3.

FIG. 8. represents divalent conjugates of one Neu5Acα6LacNAc, and one Neu5Acα6LacNAcβ3Lac, structure 28, Table 3.

FIG. 9 represents divalent conjugates of two Neu5Acα6LacNAcβ3Lac, structure 25, Table 3.

FIG. 10. Example of midproducts of enzymetic synthesis Scheme 2. The peaks at m/z 911.4, 933.3 and 949.3 represent [M+H]⁺ (massa plus proton), [M+Na]⁺ and [M+K]⁺, respectively of the oligosaccharide GlcNAcβ3[Galβ4GlcNAcβ6]Galβ4Glc and the peaks at m/z 1224.4 and 1240.4 represent [M+Na]⁺ and [M+K]⁺ of GlcNAcβ3[Neu5Acα6Galβ4GlcNAcβ6]Galβ4Glc. The sialylated product can be effectively purified by ion exchange chromatography. MALDI-TOF mass spectrometry linear positive mode.

FIG. 11. Example of midproducts of enzymetic synthesis Scheme 2. The peak at m/z 933.3 represent [M+Na]⁺ of the oligosaccharide GlcNAcβ3[Galβ4GlcNAcβ6]Galβ4Glc and the peak at m/z 1095 represent a putative hexasaccharide impurity originating from the starting material removable by chromatography or during following reaction steps. MALDI-TOF mass spectrometry reflector positive mode.

FIG. 12. Example of midproducts of enzymetic synthesis Scheme 1. The peak at m/z 1362 represent [M−H]⁻ of the oligosaccharide Galβ4GlcNAcβ6[Neu5Acα6Galβ4GlcNAcβ3]Galβ4Glc. MALDI-TOF mass spectrometry reflector negative mode.

FIG. 13. Example of midproducts of enzymetic synthesis Scheme 1. The marked peaks represent various ion forms [M+(H, Na, K, K+Na, or 2K)]⁺ of the oligosaccharide GlcNAcβ3Galβ4GlcNAcβ6[Neu5Acα6Galβ4GlcNAcβ3]Galβ4Glc. MALDI-TOF mass spectrometry linear positive mode.

FIG. 14. Example of midproducts of enzymetic synthesis Scheme 1. The marked peak represent various ion forms [M−H]⁻ of the oligosaccharide Galβ4GlcNAcβ3Galβ4GlcNAcβ6[Neu5Acα6Galβ4GlcNAcβ3]Galβ4Glc. MALDI-TOF mass spectrometry linear negative mode.

FIG. 15. Example analysis of the end product 7. The marked peak represent various ion forms [M−H]⁻, [M+Na−H]⁻, and [M+K−H]⁻ of the oligosaccharide Neu5Acα6Galβ4GlcNAcβ3Galβ4GlcNAcβ3[Neu5Acα6Galβ4GcNAcβ6]Galβ4Glc. MALDI-TOF mass spectrometry linear negative mode.

FIG. 16. Abbreviations used in Schemes 1-6.

FIG. 17. ELISA assay of serum antibodies of test subjects 1-6 (S1-6) on malcimide immobilized peptides 1 and 2 and peptide HA11, Y-axis indicate the absorbance units.

FIG. 18. ELISA assay of serum antibodies of test subjects 1-6 (S1-6) on steptavidin immobilized peptides 1-3, Y-axis indicate the absorbance units.

Scheme 1. An enzymatic synthesis scheme for a branch specifically constructed oligosaccharide structure.

Scheme 2. An enzymatic synthesis scheme for a branch specifically constructed oligosaccharide structure.

Scheme 3. An enzymatic synthesis scheme for a branch specifically constructed oligosaccharide structure.

Scheme 4. An enzymatic synthesis scheme for a branch specifically constructed oligosaccharide structure.

Scheme 5. An enzymatic synthesis scheme for a branch specifically constructed oligosaccharide structure.

Scheme 6. An enzymatic synthesis scheme for a branch specifically constructed oligosaccharide structure.

### DETAILED DESCRIPTION OF THE INVENTION

The invention reveals novel peptide vaccine compositions, and peptides for analysis and development of antibodies, when the peptides are derived from carbohydrate binding sites of carbohydrate binding proteins (lectins/adhesions) of pathogens, in a preferred embodiment human pathogens such as influenza virus.

The preferred carbohydrate binding sites are carbohydrate binding sites of pathogens comprising large carbohydrate binding sites involving binding to multiple monosaccharide units, more preferably including binding sites for two sialic acid structures. The invention is specifically directed to use of several peptides derived from carbohydrate binding site(s) of a pathogen surface protein, preferably from different parts of the carbohydrate binding site, more preferably from two different sialic acid epitope binding sites or one sialic acid binding site and conserved/semiconserved carbohydrate binding site bridging the sialic acid binding sites.

The invention reveals that conserved or semiconserved amino acid residues form reasonably conserved peptide epitopes at the binding sites of sialylated glycans, preferably binding sites disclosed in the invention. The preferred peptides are derived from the hemagglutinin protein of human influenza protein. It is realized that these epitopes can be used for development of antibodies and vaccines.

The useful antigenic peptides disclosed in the invention are available on the surface of the pathogen, preferably on viral surface.

The peptides which are 1) derived from the carbohydrate binding site (or in a separate embodiment more generally from a conserved binding site of low molecular weight ligand) and which are 2) present on the surface of a pathogen are referred here as “antigen peptides”.

### Length of Preferred Epitopes of Antigen Peptides

“Short epitopes” of about 5 amino acid residues. Prior art has studied long peptides covering usually 10-20 amino acid residues. The present invention is directed to peptide epitopes exposed on the viral surface. The epitopes are selected to direct immune reactions to conserved linear epitopes. The epitopes are relatively short about 5 amino acid residues long, preferably 3 to 8 amino acid residues, more preferably 4 to 7 aminoacid residues, most preferably 5 to 6 amino acid residues long.

Additional residues to improve presentation. The about 5 amino acid residues long sequence preferred amino acid epitopes may be further linked to assisting structures. The preferred assisting structures includes amino acid residues elongating the short epitope by residues giving additional binding strength and/or improving the natural type presentation of the short epitopes. Additional residues may be included at amino terminal and/or carboxy terminal side of the short epitopes. Preferably there are 1-3 additional residues on either or both side of the short epitopes, more preferably 1-2 additional residues.

Conformational structures. The preferred short epitopes and/additional residues may further include conformational structures to improve the three dimensional presentation of the short epitope. The preferred conformational structures includes
A) conformational conjugation structures, such as a chemical linker structure improving the conformation of the peptides
B) single amino acid residue presentation improvement, which preferably includes replacement of non-accessible single residue, with a non-affecting structure such as linkage to a carrier or replacement by alanine or glycine residue.

The conformational structures include natural 3D analogues of the epitopes on the viral surfaces:

1) disulfide bridge mimicking structures, which may include natural disulfide bridges or chemical linkages linking cysteine residues to carrier
2) bridging structures including bridging structures
forming a loop for natural type representation
bridging between two peptide epitopes

Recognition by immune system. The peptides are recognizable by the immune system of the patient and can induce immune reaction against the peptides. The immune reaction such as an antibody reaction and/or cell mediated immune reaction can recognize the peptide epitope on the surface of the virus and diminish or reduces its activity in causing disease. In a preferred embodiment the invention is specifically directed to peptides recognized by antibodies of a patient and development of such peptides to vaccines.

Preferred immune recognition by relevant species such as human and/or pandemic animal species. It is realized that most of the prior art has studied the immunoreactivity of various, in general long, peptide epitopes with regard to species used for immunological experiments such as mice, rats, rabbits or guinea pigs. It is realized that studies with regard to these immune systems is not relevant with regard to the human disease and there is multitude of results supporting this fact. The results have been very varying and does not reveal useful short epitopes with regard to human immune system.

The present invention is directed to analysis of the effect of the antigen peptides in animal species from which influenza infection is known to effectively spread to humans (see U.S. patent application No. 20050002954). Preferred animal species are avian species and/or pig. The preferred avian species includes poultry animals such as chicken and ducks, and wild bird species such as ducks, swans and other migratory water birds spreading influenza virus.

The present invention revealed that the short peptide epitopes are useful against viruses spreading from the relevant species to human patients. It was realized that the epitopes are recognizable on the surfaces of viruses and antibodies binding to peptides would block the carbohydrate binding sites of the viruses.

Screening of antibodies. The invention is directed to screening methods to reveal natural antibodies binding to peptides, preferably peptides derived from carbohydrate binding sites of human pathogens especially carbohydrate binding sites of pathogens comprising large carbohydrate binding sites involving binding to multiple monosaccharide units, more preferably including binding sites for two sialic acid structures. Preferably the invention is directed to screening of human natural antibody sequences against peptides derived from viruses or bacteria, more preferably against carbohydrate binding sites of influenza viruses.

It is realized that antibodies may be screened by affinity methods involving binding of antibodies to the peptide epitopes. The peptide epitopes may be conjugated to solid phase for the screening, preferably for screening of human antibodies. In a preferred embodiment the peptides are screened from blood, blood cells or blood derivative such as plasma or serum of a patient. In another embodiment the antibodies are screened from a phage display library derived from blood cells of a patient or several patients or normal subjects, preferably expected to have immune reaction and antibodies against the peptides disclosed in the invention.

Screening of peptides. The invention is further directed to screening of the preferred peptide epitopes and analogous peptides and conjugates thereof against human immune reactions for development of the optimal vaccines and antibody development products.

The invention is further directed to further screening of, and binding analysis of peptides, which are recognized by patients immune system preferably by natural antibodies of a patient. The invention is directed to screening methods to reveal further peptides derived from carbohydrate binding proteins (adhesions/lectins) of human pathogens, especially carbohydrate binding sites of pathogens comprising large carbohydrate binding sites involving binding to multiple monosaccharide units, more preferably including binding sites for two sialic acid structures.

Preferred types of influenza viruses. The influenza viruses are preferably viruses involving risk for human infection, including human influenza viruses, and/or potentially human infecting pandemic influenza viruses such as avian influenza viruses. More specifically the preferred virus is influenza A, influenza B and influenza C viruses, even more preferably influenza A or B, and most preferably influenza A. Preferably influenza A is a strain infecting or potentially infecting humans such as strains containing hemagglutinin type H1, H2, H3, H4, or H5.

### Preferred Peptides or Groups of Peptides for Influenza Viruses

The invention is directed to specific peptide epitopes and variants thereof for treatment of influenza (including prophylactic or preventive treatments). The invention is specifically directed to specific peptide epitopes and groups thereof for treatment of specific subtypes of influenza such as influenzas involving hemagglutinin types H1, H2, H3, H4, or H5, more preferably H1, H2, H3, or H5.

### Preferred Conserved Amino Acid Epitopes Antigen Peptides, for Vaccine or Antibody Development

The present invention is preferably directed to following peptide epitopes, and any linear tripeptides or tetrapeptides derivable thereof or combinations thereof for vaccine and antibody development, preferably directed for the treatment of human influenza. The invention is further directed to elongated versions of the peptides containing 1-3 amino acid residues at N- and/or C-terminus of the peptide. The numbering of the peptides is based on the X31-hemagglutinin if not other wise indicated. This indicated corresponding position of the peptides in three dimensional structure of the hemagglutinin and same position with regard to conserved cysteine bridge for Peptide 1 and Peptide 2 and presence in the loop structure as described for Peptide 3.

The invention is specifically directed to sequencing and analysing corresponding peptides from new influenza strains, because the viruses have tendency to mutate to avoid human immune system. The invention further revealed that it is possible to use several peptides according to the invention. Persons resistant to influenza virus had antibodies against 2 or 3 peptides. The invention is directed to vaccines against single type of influenza H1, H2, H3, H4 or H5.

The invention is further directed to peptide compositions comprising at least one peptide, more preferably at least two and most preferably at least three peptide, against at least two, more preferably at least three, different hemagglutinin subtypes, preferably against H1, H3, and/or H5. In a specific embodiment the invention is directed to peptides of H5-hemagglutinins aimed for treatment or prevention of avian influenza.

It is further realized that similar peptides may be derived from other influenza virus hemagglutinins. The invention is specifically directed to defining structurally same peptide positions from influenza B, Influenza C and other hemaglutinin subtypes such as H6, H7, H8, or H9.

It is further realized that the peptides may be used in combination with known and published/patented peptide vaccines against influenza and/or other influenza drug. The invention is specifically directed to the use of the vaccines together with hemagglutinin binding inhibiting molecules according to the invention, preferably divalent sialosides. The invention is further directed to the use of the molecules together with neuraminidase inhibitor drugs against influenza such as Tamiflu of Roche or Zanamivir of GSK or Peramivir of Biocryst or second generation neuraminidase inhibitors such as divalent ones developed by Sankyo and Biota

The peptides are preferably aimed for use as conjugates as polyvalent and/or immunomodulator/adjuvant conjugates. The preferred epitopes do not comprise in a preferred embodiment additional, especially long amino acid sequences, preferably less than 7 amino acid, more preferably less than 5, more preferably less than 3 and most preferably less 1 or 0 additional amino acid residues, directly continuing from the original hemagglutinin sequence

Many peptide vaccines have been described against influenza virus. These contain various peptides of the virus usually conjugated to carriers, or other immunogenic peptides and/or adjuvants and further including adjuvant molecules to increase antigenicity.

Person skilled in the art can determine the corresponding amino acid position from other influenza hemagglutinins in relation to most conserved amino acid residues and/or position of disulfide bridges and design similar peptides containing 1-3 different, more preferably 1-2 different amino acid residues, most preferably only one different amino acid residue. Design of analogs and elongated variants of the peptides involves analysis of the surface presentation of the peptides, so that these would be accessible for analytic/diagnostic and/or therapeutic recognition by specific binding agents, such as antibodies, peptides (such as phage display peptides), combinatorial chemistry libraries and/or aptamers.

### Preferred Hemagglutinin Peptides

Region of Amino Acid at Positions of about 210- to 230 of Hemagglutinin

Similarity is observed between influenza A viruses for example as partial sequence KVR and isoforms in hemagglutinin type H1 sequences, WVR in H3 and KVN in H5. The region is favoured because presence on the surface of the virus available for immune recognition and because antibodies binding to the region would interfere with carbohydrate binding of the virus. The peptides form a conserved loop type epitope which can be further used for production of cyclic peptides.

### Preferred KVR-Region Peptides of H1 Similar Peptides

The conserved amino acid (from amino terminus to C-terminus) Lys222-Val223-Arg224 KVR homologous to WVR-region of X31 hemagglutinin forms an excellent target for recognition of influenza virus. This relatively conserved sequence is present e.g. in the sequence RPKVRDQ of A/South Carolina/1/1918 (H1N1), also known as “Spanish Flu”-hemagglutinin. The peptide was modelled as an exposed sequence on the surface of the virus. The peptide sequence is preserved in hundreds human influenza A viruses. The region comprise a tripeptide Lys222-Val223-Arg224 (KVR), which is a preferred peptide epitope according to the invention and present in longer peptide epitopes. Preferred peptide epitopes includes heptapeptide RPKVRDQ and further includes pentapeptides: RPKVR, PKVRD, KVRDQ and hexapaptides RPKVRD and PKVRDQ. The proline is preferred as an amino acid affecting the conformation of the peptide, the D-residues is preferred as a semi-conserved amino acid residue, it may be replaced by similar type amino acid residue

### Conserved Peptide 3 Region of Hemagglutinin 2, H2

The invention revealed that human hemagglutinin 2 also contains conserved Peptide I region the examples of the sequences includes RPFVNGQ AND RPKVNGL at position 99-105, see Table 8, the epitope comprises additional aminoacid residues K and E-especially at N-terminal side, with consensus sequence RPXVNG or PXVNG, RPXVN, RPXV, PXVN, XVNG, RPX, PXV, XVN wherein X is any aminoacid preferably E or K

### Preferred WVR-Region Peptides of H3 Similar Peptides

The conserved amino acid (from amino terminus to C-terminus) Trp222-Val223-Arg224 WVR of region B of X31 hemagglutinin forms another excellent target for recognition of influenza virus. The peptide was modelled as an exposed sequence on the surface of the virus. The peptide sequence is preserved in more than hundred human influenza A viruses. The region comprise a tripeptide Lys222-Val223-Arg224 (WVR), which is a preferred peptide epitope according to the invention and present in longer peptide epitopes. Preferred peptide epitopes includes heptapeptide RPWVRGL and further includes pentapeptides: elongated variants pentapeptides, RPWVR, PWVRG, WVRGL and hexapaptides RPWVRG and PWVRGL. The proline is preferred as an amino acid affecting the conformation of the peptide, the L-residues is preferred as a semi-conserved amino acid residue, it may be replaced by similar hydrophobic amino acid residue. The preferred variants include ones where W is replaced by R-residue.

### Preferred KVN-Region Peptides of H5 Similar Peptides

The conserved amino acids Lys222-Val223-Asn224 (KVN, from amino terminus to C-terminus) observable for example from H5-hemagglutinins A/Vietnam/1203/2004 (H5N1) or A/duck/Malaysia/F119-3/97 (H5N3), corresponding to conserved region B of X31 hemagglutinin forms a further target for recognition of influenza virus. The peptide was modelled as an exposed sequence on the surface of the virus. The peptide sequence is preserved in more than hundred human influenza A viruses.

Preferred peptide epitopes further includes elongated variants peptides being the heptapeptide RPKVNGQ, hexapeptides RPKVNG, and PKVNGQ, pentapeptides RPKVN, PKVNG, KVNGQ, RPKVNG, and PKVNGQ. The penta- to hepta peptides all includes the preferred tripeptide structure KVN. The invention is further directed to tetrapeptides RPKV, PKVN, including the preferred subepitope KV and KVNG and VNGQ including preferred subepitope VN. The proline is preferred as an amino acid affecting the conformation of the peptide, it may be replaced by similar type amino acid residue.

The invention is specifically directed to consensus of Peptide 3 region

### RPX1VX2X3

### X₁ is K, E, R or W

### X₂ is N, or R

X3 is noting, D or G.
Cyclic Peptides of the Region about 210-230

The invention is further directed cyclic peptides including the preferred peptide epitopes above. Most preferably a natural type heptapeptides RPKVRDQ, RPWVRGL, RPKVNGQ linked to a cyclic peptide by residues X and Y:

X—H7-Y,

wherein H7 is the heptapeptide and
X is group forming cyclic structure with group Y,

In a preferred embodiment X and Y are Cys-residues forming disulfide bridge With each other.

The groups X and Y include preferably

pair of specifically reactive groups
such as amino-oxy (—R—O—NH2) and reactive carbonyl such as aldehyde or ketone; azide (—R—N═NH2) and reactive carbonyl such as aldehyde or ketone
Region of Amino Acid at Positions of about 85- to about 100/98-106

Similarity is observed between influenza A viruses within a region corresponding to the amino acids located before cysteine 97 in the structure of H3 hemagglutinin X31. The region is favoured because presence on the surface of the virus available for immune recognition and because antibodies binding to the region would interfere with carbohydrate binding of the virus. The region is mainly semiconserved, there is similar variants of the sequences, which are relatively well conserved within each hemagglutinin type.

### Preferred TSNSENGT(C)-Region of H1 Type Viruses

The amino acid residues before the X31Cys97 equivalent are located e.g. at positions 86-93 of A/South Carolina/1/1918 (H1N1) with sequence TSNSENGT(C) or NSENGT(C). Especially the region TSESEN, more preferably SESEN is well exposed on the surface of the virus, while the conformation of the last two amino acid residues GT in the region are less well exposed. In a preferred embodiment one or both of the C-terminal residues and optionally also the Cys-residue are included as “additional residues” to achieve optimal presentation and/or conformation. Preferred variants includes peptides NPENGT(C), PNPENGT(C) and TPPENGT(C); NSENGI(C), PNSENGIC(C) and TPNSENGIC (C). The preferred consensus sequence includes

NX₁ENGX₂(C), and shorter variants ENGX₂(C), NX₁EN,
wherein X₁ and X₂ are variable residues, preferably ones described above and cysteine (C) may be present or absent, preferably present, more preferably as thiol conjugate;

### and ENG.

### Conserved Peptide 1 Region of Hemagglutinin 2, H2

The invention revealed that human hemagglutinin 2 also contains conserved Peptide 1 region the examples of the sequences includes NPRNGLC AND NPRYSLC at position 99-105, see Table 8, the epitope comprises additional aminoacid residues K and E—especially at N-terminal side, with consensus sequence NPR or NPRXXL(C), PRXXL(C), RXXL(C), wherein cysteine (C) may be present or absent, preferably present, more preferably as thiol conjugate;

### Preferred SKAFSN(C)-Region Peptides of H13 Type Viruses

The conserved aminoacid (from amino terminus to C-terminus) Ser91-Lys92-Ala91-Phe94-Ser95-Asn96-Cys97 (SKAFSNC) as presented in human H3-hemagglutinin belong to, at least partially conserved, and exposed and available region. The peptide sequence is preserved in more than hundred human influenza A viruses H3. Preferred peptide epitopes further includes elongated varianta AFSN, SKAFSN, SKAFS, and SKAF. In a preferred embodiment one or both of the C-terminal residues and optionally also the Cys-residue are included as “additional residues” to achieve optimal presentation and/or conformation.

Recent A-influenza viruses contain especially preferred variants wherein F is replaced by Y(tyrosine): AYSN, SKAYSN, SKAYS, and SKAY. Furthermore variant wherein Lysin is replaced by T (threonine) are preferred: STAYSN, STAYS, and STAY, which are also present in recent influenza viruses.

Preferred KXNPVNXL(C)-Region of H5 Type Viruses

The amino acid residues before the X31Cys97 equivalent are located e.g. at positions 99-106 of A/duck/Malaysia/F119-3/97 (H5N3) with sequence KDNPVNGL(C) and at positions of 98-105 of A/Viet Nam/1203/2004 (H5N1) with the sequence KANPVNDL(C). Especially the region KXNPVN, more preferably XNPVN is well exposed on the surface of the virus, while the conformation of the last two amino acid residues, XL, in the region are less well exposed. In a preferred embodiment one or both of the C-terminal residues and optionally also the Cys-residue are included as “additional residues” to achieve optimal presentation and/or conformation.

Region of Amino Acid at Positions of about 130- to about 140

Similarity is observed between influenza A viruses within a region corresponding to the amino acids located before cysteine 139 in the structure of H3 hemagglutinin X31, and in a preferred embodiment also including Cys139 equivalent and few following amino acid residues. The region is favoured because presence on the surface of the virus available for immune recognition and because antibodies binding to the region would interfere with carbohydrate binding of the virus. The region is mainly semiconserved, there is similar variants of the sequences, which are relatively well conserved within each hemagglutinin type.

### Preferred TTKGVTAA(C)-Region of H1 Type Viruses

The amino acid residues before the hemagglutinin X31-Cys139 equivalent are located e.g. at positions 132-139 of A/South Carolina/1/1918 (H1N1) with sequence TTKGVTAA(C). The preferred exposed sequence includes the Cys residue and 1-4 amino acid residues after it. In a preferred embodiment one or two additional residues of the C-terminal and/or N-terminal residues and optionally also the Cys-residue are included as “additional residues” to achieve optimal presentation and/or conformation.

The H1 Peptide 2 is preferred at position 148-153 in sequences containing signal sequence see Table 6, see Table 8, the Table describes additional aminoacids TK, TN, and TR at aminoterminal side and preferred additional sequences as Peptide 2b and its N-terminal aminoacids and di- to tetrapeptides, the preferred core epitopes are

### GVTAA(C) and GVTAS(C), and

### VTAA(C) and VTAS(C),

### VTAX(C),

cysteine (C) may be present or absent, preferably present, more preferably as thiol conjugate.

### Conserved Peptide 2 Region of Hemagglutinin 2, H2

The invention revealed that human hemagglutinin 2 also contains conserved Peptide 1 region the examples of the sequences includes SQGCAV AND SWACAV, see Table 8, the epitope comprises additional aminoacid residues at N-terminal side, preferably TTGG, or TGG, OR GG, with consensus sequence TTGGSXXCAV or

### GSXX(C)AV

### GSX₁X₂(C)A

GSX₁X₂(C), wherein
X₁X₂ are any aminioacid preferably X₁ is Q and W; and X₂ is A or G, respectively cysteine (C) may be present or absent, preferably present, more preferably as thiol conjugate, when C is absent in the middle of chain it is replaced by glycine or alanine preferably by glycine.

### Preferred GGSNA-Region Peptides of H3 Type Viruses

The conserved amino acid (from amino terminus to C-terminus) Gly134-Gly135-Ser136-Asn137-Ala138 of region A (GGSNA) of region B of X31 hemagglutinin forms an excellent target for recognition of influenza virus. The peptide was modelled as an exposed sequence on the surface of the virus. The peptide sequence is preserved in more than hundred human influenza A viruses.

Preferred peptide epitopes further includes elongated variants such as GGSNACKRG, GSNACKRG, SNACKRG, NACKRG, GGSNACKR, GSNACKR, SNACKR, NACKR. The preferred variants includes sequences wherein N is replaced by S, or T and other variants of recent influenza viruses with 1-2 substitutions, especially aromatic aminoacid variants including tyrosine.

Other preferred sequences includes SYACKR and SSACKR and N- and C-terminally elongated variants with additional 1-3 amino acids the consensus sequences

### SX₂A(C)KR

### X₁ SX₂(C)KR

### GX₁SX₂A(C)KR

### SX₂A(C)K

### X₁SX₂(C)K

### GX₁SX₂A(C)K

### SX₂A(C)

### X₁SX₂(C)

### GX₁SX₂A(C)

Wherein X₁ is any aminoacid preferably G, T, or E
And X₂ is any amino acid preferably N, Y or S,
cysteine (C) may be present or absent, preferably present, more preferably as thiol conjugate, when C is absent in the middle of chain it is replaced by glycine or alanine preferably by glycine.

### Preferred DASSGVSSA(C)PY-Region of H5 Type Viruses

The amino acid residues before the hemagglutinin X31-Cys139 equivalent are located e.g. at positions 142-150 DASSGVSSA(C)PYNG (numbering including signal peptide) of A/duck/Malaysia/F119-3/97 (H5N3) and at positions of 142-150 of A/Viet Nam/1203/2004 (H5N1) with the sequence EASLGVSSA(C)PYQG. Especially the region (E/D)ASXGVSSA, more preferably GVSSA is well exposed on the surface of the virus. In a preferred embodiment one or both of the C-terminal residues and optionally also the Cys-residue are included as “additional residues” to achieve optimal presentation and/or conformation.

### Less-Available but Conserved Sequences

The invention reveal novel peptide epitopes, which are very conserved among influenza viruses, but less surface exposed and thus less available regular immunotherapies on cell surfaces. It is realized that presence of such peptides for example on T-cell receptors or antibodies against these are indicative of immune reaction against influenza. Studies of such immune reactions are useful for analysis of immune reactions against influenza, though such reaction may be less useful against influenza. Immune reactions are indications about the strength and direction of immune response. The analysis may be used peptide analysis of presence of influenza or other influenza diagnostics. The sequences are further useful for PCR analysis of the infection by analysis of nucleic acid sequences corresponding to the conserved peptide epitopes.

### Conserved Less-Available “Core Sequences” of Influenza A Viruses

Beside the active surface sequences the present invention revealed certain other conserved amino acid sequences present in the viruses. The less available sequences referred here as “core sequences” comprise usually large hydrophobic amino acids. Most of the sequences are conserved in larger groups of influenza viruses such as influenza A or influenza B viruses. The invention is especially directed to the analysis of the highly conserved core sequence(s) together with one or several of the antigen peptides, which are more specific for the subtype of the virus.

### (L)WG(I or V)HHP

(L)WGIHHP and (L)WGVHHP sequences correspond to X31 aminoacids (178) 179-184 and belong to the less available sequences. It does not appear on the surface of virus and would not be useful for regular vaccination use. These peptide sequences and corresponding nucleic acid sequences are, however, useful for analysis of influenza viruses. The sequences are present in practically all influenza A viruses and can be thus used for typing of viruses, especially defining presence of influenza A virus in a sample.

### The Corresponding Nucleic Acid Sequences

Preferred analytical and/or therapeutic tools include corresponding nucleic acid sequences, especially the influenza virus nucleic acid sequences coding the peptide epitopes useful for example DNA/RNA diagnostics and/or for gene therapy/RNAi-methods. Preferred diagnostic methods include known polymerase chain reaction, PCR, methods known for influenza diagnostics (see U.S. Pat. No. 6,811,971 and WO0229118). The preferred nucleic acid sequences include sequences coding aminoacid (L)WGIHHP and (L)WGVHHP corresponding to X31 aminoacids (178) 179-184 or part thereof.

### Peptide Vaccines and Use Thereof

### Peptide Vaccine Compositions

Peptides can be produced using techniques well known in the art. Such techniques include chemical and biochemical synthesis. Examples of techniques for chemical synthesis of peptides are provided in Vincent, in Peptide and Protein Drug Delivery, New York, N.Y., Dekker, 1990. Examples of techniques for biochemical synthesis involving the introduction of a nucleic acid into a cell and expression of nucleic acids are provided in Ausubel, Current Protocols in Molecular Biology, John Wiley, and Sambrook, et in Molecular Cloning, A Laboratory Manual, Cold Spring Harbor Laboratory Press, 1989.

The application discloses a method of inducing an immune response against a peptide of region B of X31 hemagglutinin. This can be accomplished by conjugating the peptide with a carrier molecule prior to administration to a subject.

In the methods disclosed herein, an immunologically effective amount of one or more immunogenic peptides derivatized to a suitable carrier molecule, e.g., a protein is administered to a patient by successive, spaced administrations of a vaccine composed of peptide or peptides conjugated to a carrier molecule, in a manner effective to result in an improvement in the patient's condition.

In an exemplary embodiment, immunogenic peptides are coupled to one of a number of carrier molecules, known to those of skill in the art. A carrier protein must be of sufficient size for the immune system of the subject to which it is administered to recognize its foreign nature and develop antibodies to it.

In some cases the carrier molecule is directly coupled to the immunogenic peptide. In other cases, there is a linker molecule inserted between the carrier molecule and the immunogenic peptide.

In one exemplary embodiment, the coupling reaction requires a free sulfhydryl group on the peptide. In such cases, an N-terminal cysteine residue is added to the peptide when the peptide is synthesized.

In an exemplary embodiment, traditional succinimide chemistry is used to link the peptide to a carrier protein. Methods for preparing such peptide:carrier protein conjugates are generally known to those of skill in the art and reagents for such methods are commercially available (e.g., from Sigma Chemical Co.). Generally about 5-30 peptide molecules are conjugated per molecule of carrier protein.

Exemplary carrier molecules include proteins such as keyhole limpet hemocyanin (KLH), bovine serum albumin (BSA), flagellin, influenza subunit proteins, tetanus toxoid (TT), diphtheria toxoid (DT), cholera toxoid (CT), a variety of bacterial heat shock proteins, glutathione reductase (GST), or natural proteins such as thyroglobulin, and the like. One of skill in the art can readily select an appropriate carrier molecule.

In a preferred embodiment an immunogenic peptide is conjugated to diphtheria toxin (DT).

In some cases, the carrier molecule is a non-protein, such as Ficoll 70 or Ficoll 400 (a synthetic copolymer of sucrose and epichlorohydrin), a polyglucose such as Dextran T 70.

Another preferred category of carrier proteins is represented by virus capsid proteins that have the capability to self-assemble into virus-like particles (VLPs). Examples of VLPs used as peptide carriers are hepatitis B virus surface antigen and core antigen (Pumpens et al., “Evaluation of and frCP virus-like particles for expression of human papillomavirus 16 E7 oncoprotein epitopes”, Intervirology, Vol. 45, pp. 24-32,2002), hepatitis E virus particles (Niikura et al., “Chimeric recombinant hepatitis E virus-like particles as an oral vaccine vehicle presenting foreign epitopes”, Virology, Vol. 293, pp. 273-280, 2002), polyoma virus (Gedvilaite et al., “Formation of Immunogenic Virus-like particles by inserting epitopes into surface-exposed regions of hamster polyomavirus major capsid protein”, Virology, Vol. 273, pp. 21-35, 2000), and bovine papilloma virus (Chackerian et al., “Conjugation of self-antigen to papillomavirus-like particles allows for efficient induction of protective autoantibodies”, J. Clin. Invest., Vol. 108 (3), pp. 415-423, 2001). More recently, antigen-presenting artificial VLPs were constructed to mimic the molecular weight and size of real virus particles et al., “Construction of artificial virus-like particles exposing HIV epitopes and the study of their immunogenic properties”, Vaccine, pp. 386-392, 2003).

A peptide vaccine composition may comprise single or multiple copies of the same or different immunogenic peptide, coupled to a selected carrier molecule. In one aspect of this embodiment, the peptide vaccine composition may contain different immunogenic peptides with or without flanking sequences, combined sequentially into a polypeptide and coupled to the same carrier. Alternatively, immunogenic peptides, may be coupled individually as peptides to the same or a different carrier, and the resulting immunogenic peptide-carrier conjugates blended together to form a single composition, or administered individually at the same or different times.

For example, immunogenic peptides may be covalently coupled to the diphtheria toxoid (DT) carrier protein via the cysteinyl side chain by the method of Lee A. C. J., et al., 1980, using approximately 15-20 peptide molecules per molecule of diphtheria toxoid (DT).

In general, derivatized peptide vaccine compositions are administered with a vehicle. The purpose of the vehicle is to emulsify the vaccine preparation. Numerous vehicles are known to those of skill in the art, and any vehicle which functions as an effective emulsifying agent finds utility in the present invention. One preferred vehicle for administration comprises a mixture of mannide monooleate with squalane and/or squalene. Squalene is preferred to squalane for use in the vaccines of the invention, and preferably the ratio of squalene and/or squalane per part by volume of mannide monooleate is from about 4:1 to about 20:1.

To further increase the magnitude of the immune response resulting from administration of the vaccine, an immunological adjuvant is included in the vaccine formulation. Exemplary adjuvants known to those of skill in the art include water/oil emulsions, non-ionic copolymer adjuvants, e.g., CRL 1005 (Optivax; Vaxcel Inc., Norcross, Ga.), aluminum phosphate, aluminum hydroxide, aqueous suspensions of aluminum and magnesium hydroxides, bacterial endotoxins, polynucleotides, polyelectrolytes, lipophilic adjuvants and synthetic muramyl dipeptide (norMDP) analogs. Preferred adjuvants for inclusion in an peptide vaccine composition for administration to a patient are norMDP analogs, such as N-acetyl-nor-muranyl-L-alanyl-D-isoglutamine, N-acetyl-muranyl-(6-O-stearoyl)-L-alanyl-D-isoglutamine, and N-Glycol-muranyl-L.alphaAbu-D-isoglutamine (Ciba-Geigy Ltd.). In most cases, the mass ratio of the adjuvant relative to the peptide conjugate is about 1:2 to 1:20. In a preferred embodiment, the mass ratio of the adjuvant relative to the peptide conjugate is about 1:10. It will be appreciated that the adjuvant component of the peptide vaccine may be varied in order to optimize the immune response to the immunogenic epitopes therein.

Just prior to administration, the immunogenic peptide carrier protein conjugate and the adjuvant are dissolved in a suitable solvent and an emulsifying agent or vehicle, is added.

Suitable pharmaceutically acceptable carriers for use in an immunogenic proteinaceous composition of the invention are well known to those of skill in the art. Such carriers include, for example, phosphate buffered saline, or any physiologically compatible medium, suitable for introducing the vaccine into a subject.

Numerous drug delivery mechanisms known to those of skill in the art may be employed to administer the immunogenic peptides of the invention. Controlled release preparations may be achieved by the use of polymers to complex or absorb the peptides or antibodies. Controlled delivery may accomplished using macromolecules such as, polyesters, polyamino acids, polyvinyl pyrrolidone, ethylenevinylacetate, methylcellulose, carboxymethylcellulose, or protamine sulfate, the concentration of which can alter the rate of release of the peptide vaccine.

In some cases, the peptides may be incorporated into polymeric particles composed of e.g., polyesters, polyamino acids, hydrogels, polylactic acid, or ethylene vinylacetate copolymers. Alternatively, the peptide vaccine is entrapped in microcapsules, liposomes, albumin microspheres, microemulsions, nanoparticles, nanocapsules, or macroemulsions, using methods generally known to those of skill in the art.

### Vaccination

The vaccine of the present invention can be administered to patient by different routes such as intravenous, intraperitoneal, subcutaneous, intramuscular, or orally. A preferred route is intramuscular or oral. Suitable dosing regimens are preferably determined taking into account factors well known in the art including age, weight, sex and medical condition of the subject; the route of administration; the desired effect; and the particular conjugate employed (e.g., the peptide, the peptide loading on the carrier, etc.). The vaccine can be used in multi-dose vaccination formats.

It is expected that a dose would consist of the range of to 1.0 mg total protein. In an embodiment of the present invention the range is 0.1 mg to 1.0 mg. However, one may prefer to adjust dosage based on the amount of peptide delivered. In either case these ranges are guidelines. More precise dosages should be determined by assessing the immunogenicity of the conjugate produced so that an immunologically effective dose is delivered. An immunologically effective dose is one that stimulates the immune system of the patient to establish a level immunological memory sufficient to provide long term protection against disease caused by infection with influenza virus. The conjugate is preferably formulated with an adjuvant.

The timing of doses depend upon factors well known in the art. After the initial administration one or more booster doses may subsequently be administered to maintain antibody titers. An example of a dosing regime would be a dose on day 1, a second dose at or 2 months, a third dose at either 4, 6 or 12 months, and additional booster doses at distant times as needed.

A patient or subject, as used herein, is an animal. Mammals and birds, particularly fowl, are suitable subjects for vaccination. Preferably, the patient is a human. A patient can be of any age at which the patient is able to respond to inoculation with the present vaccine by generating an immune response. The immune response so generated can be completely or partially protective against disease and debilitating symptoms caused by infection with influenza virus.

### Evaluation of the Immune Response

In one aspect, the invention provides a means for classifying the immune response to peptide vaccine, e.g., 9 to 15 weeks after administration of the vaccine; by measuring the level of antibodies against the immunogenic peptide of the vaccine.

The invention thus includes a method of monitoring the immune response to the peptide(s) by carrying out the steps of reacting a body-fluid sample with said peptide(s), and detecting antibodies in the sample that are immunoreactive with each peptide. It is preferred that the assay be quantitative and accordingly be used to compare the level of each antibody in order to determine the relative magnitude of the immune response to each peptide.

The methods of the invention are generally applicable to immunoassays, such as enzyme linked immunosorbent assay (ELISAs), radioimmunoassay (RIA), immunoprecipitation, Western blot, dot blotting, FACS analyses and other methods known in the art.

In one preferred embodiment, the immunoassay includes a peptide antigen immobilized on a solid support, e.g., an ELISA assay. It will be appreciated that the immunoassay may be readily adapted to a kit format exemplified by a kit which comprises: (A) one or more peptides of the invention bound to a solid support; (B) a means for collecting a sample from a subject; and (C) a reaction vessel in which the assay is carried out. The kit may also comprise labeling means, indicator reaction enzymes and substrates, and any solutions, buffers or other ingredients necessary for the immunoassay.

### Diagnosis of Influenza Infection

The present invention is also directed to diagnosis of an influenza infection. General methods for diagnosis of an influenza infection are well known to a skilled artisan and are disclosed for instance in U.S. Pat. No. 6,811,971. The present invention provides a method of identifying influenza virus in a biological sample by (a) contacting the biological sample with a nucleic acid primers amplifying the part of virus genome encoding for the divalent sialoside binding site of the X31-hemagglutinin protein as disclosed below under conditions allowing polymerase chain reaction; and (b) determining the sequence of the amplified nucleic acid in the biological sample, to thereby identify the presence and type of influenza virus. Alternatively, the presence of influenza virus can be detected by (a) contacting the biological sample with an antibody or antibody fragment specifically recognizing the divalent sialoside binding site of the X31-hemagglutinin protein as disclosed below; and (b) detecting immunocomplexes including said antibody or antibody fragment in the biological sample, to thereby identify the presence and type of influenza virus in the biological sample.

### Divalent Sialoside Polylactosamines and Spacer Comprising Analogs Thereof

The present inventors found special divalent poly-N-acetylactosamine sequences capable of binding a specific region of human influenza virus. The inventors further found out that specific spacer modified divalent sialosides, preferably spacer modified oligosaccharides, can be used for binding and inhibition of influenza virus. It is further realized that the specific binding region to which the oligosaccharide sequence binds on the surface of hemagglutinin, or part thereof, can be used as target for drug design. The data about the complex between the divalent sialosides according can be further used for design of further analogs for the sialosides. The inventors further found out that specific spacer modified sialosides, preferably spacer modified oligosaccharides, can be used for binding and inhibition of influenza virus.

The divalent sialosides according to the present invention bind to hemagglutinin proteins of influenza viruses, preferably hemagglutinins of human infecting viruses or bird infecting viruses, more preferably human infecting viruses, even more preferably influenza A-type viruses.

The present invention is specifically directed to search of divalent sialoside substances when one sialic acid residue of the divalent structure is docked to the primary sialic acid binding site of the hemagglutinin and the binding site for the other sialic acid structure is searched docking the other sialic acid terminal with positively charged aminoacid residues on the surface of the hemagglutinin within the range of the structure of the spacer structure, preferably the spacer structure according to the invention. Preferably the docking involves optimizing the conformation of the spacer structure on the surface of the hemagglutinin.

The inventors found that there is differences in binding of various divalent sialosides and various influenza virus strains or influenza virus types. The present invention is further directed to combinations of divalent sialosides according to the invention or at least one divalent sialoside according to the present invention and other bioactive divalent sialosides described previous inventions including the previous inventions about poly-N-acetyllactosamine structures including (FI20001477, WO0197819), such as branched structures NeuNAcα3/6Galβ4GlcNAcβ3(NeuNAcα3/6Galβ4GlcNAcβ6)GalβR wherein R is an organic residue or a monosaccharide structures preferably glucose, or glucoside or GlcNAc or glycoside thereof preferably β4-linked from the reducing end galactosyl residue.

The present invention is specifically directed to divalent alpha-sialoside wherein the distance between the sialic acid residues is between about 25 Å and 55 Å for use in binding of human influenza virus. When an oligosaccharide is used, preferably larger than disaccharide, more preferably larger than a trisaccharide, the preferred length of the sialoside may be up to about 65 Å, more preferably up to about 60 Å The present invention is preferably directed to sialosides when the distance between the sialic residues is between about 26 Å and about 54 Å. In preferred embodiments the distance between silica acids is between 26 Å and 50 Å. More preferably the distance between sialic acid carboxylic acid groups is more than about 30 Å, more preferably more than about 35 Å. In a preferred embodiment the distance is about 36 Å, or about 49 Å or about 59 Å; and in another preferred embodiment the invention is directed to an oligosaccharide comprising structure between 35 and 60 Å. In a preferred embodiment the preferred ranges are limited under 50 Å.

The present invention is preferably directed to oligosaccharide based divalent conjugates. The oligosaccharide based divalent sialosides have in a preferred embodiment the same distances between sialic acid residues as described in general by the invention. The larger oligosaccharide however can have additional binding interactions in specific binding sites as described by examples by modelling and may be require longer spacers because of the conformation and/or direction of the oligosaccharide sequences in the binding sites. The present invention is specifically directed to preferred oligosaccharide sequences linked by a spacer length about 8-16 Å or comprising about 8 to 16 atomic bonds between the oligosaccharide sequences, more preferably the present invention is directed to spacer of about 9-15 Å or atomic bonds between the oligosaccharide sequences and even more preferably 10-15 Å or atomic bonds and most preferably the spacers between the oligosaccharide sequences has a length of about 13-15 Å or 13-15 atomic bonds between the ring structures of the oligosaccharide sequences. In a preferred embodiment the preferred spacer lengths described above are used for trisaccharides, tetrasaccharides and or pentasaccharides, in a preferred embodiment spacer length of about 5 Å or atomic bonds are added when per one disaccharide used in the conjugate. The spacer length reflect the actual extended conformation length of the spacer and not the distance between the oligosaccharide rings in bound conformation,

The present invention therefore directed to divalent alpha6-sialylated oligosaccharide structures according to the formula SAα6Galβ4[Glc(NAc)₀ₒᵣ₁)]₀ₒᵣ₁{(β3Gal[β4Glc(NAc)₀ₒᵣ₁]₀ₒᵣ₁}₀ₒᵣ₁ and/or SAα3Galβ4[Glc(NAc)_{0or1)]}₀ₒᵣ₁{β3Gal[β4Glc(NAc)₀ₒᵣ₁]₀ₒᵣ₁}₀ₒᵣ₁ linked with spacer to form dimeric or oligomeric or polymeric structures having specific distances between two active sialylated terminal structures, wherein SA is sialic acid preferably N-acetylneuraminic acid, Neu5Ac. The sialic acid maybe also any known analogue of sialic acid, preferably a sialic acid capable of binding to hemagglutinin. In a preferred embodiment the divalent sialoside contain at least one sialic acid analogue or derivative, more preferably a sialic acid analogue or derivative known to bind to the primary sialic acid binding site of hemagglutinin is included in the sialoside. The sialic acid oligosaccharide sequence is in a preferred embodiment represented by the formula

SAαXGalβ4[Glc(NAc)ₙ₁)]ₙ₂{β3Gal[β4Glc(NAc)ₙ₃]ₙ₄}ₙ₅

Wherein X is linkage position 3 or 6
wherein SA is sialic acid or sialic acid analogue or derivative, preferably N-acetylneuraminic acid, Neu5Ac
and n1, n2, n3, n4 and n5 are 0 or 1 independently, with the provision that when n2 is 0 the also n5 is 0 and all [ ], ( ), and { } represent structures which are either present or absent. In a preferred embodiment two different oligosaccharide sequences are used. Preferred lengths of oligosaccharide sequences include disaccharides, trisaccharides, tetrasaccharides and pentasaccharides, more preferably in combination disaccharide and tetrasaccharide or trisaccharide and pentasaccharide or two disaccharides or two trisaccharides. In a preferred embodiment at least one sialic acid is α6-linked, more preferably both sialic acids are α6-linked. The saccharides linked by oxime bonds according to the invention have both open chain double bond forms and ring closed glycosidic forms, allowing presentation of various oligosaccharide lengths.

Preferred oligosaccharide sequences includes α6-sialyl oligosaccharide sequences:

### SAα6Galβ

### SAα6Galβ4Glc

### SAα6Galβ4GlcNAc

### SAα6Galβ4GlcNAcβ3Galβ4Glc

### SAα6Galβ4GlcNAcβ3Galβ4GlcNAc

In a preferred embodiment two a6-sialyl oligosaccharide sequences according to the formula are used. In a preferred embodiment the oligosaccharide sequences are used in combination with an oligosaccharide containing at least one α6-linked oligosaccharide sequence.

Preferred oligosaccharide sequences includes α3-sialyl oligosaccharide sequences:

### SAα3Galβ

### SAα3Galβ4Glc

### SAα3Galβ4GlcNAc

### SAα3Galβ4GlcNAcβ3Galβ4Glc

### SAα3Galβ4GlcNAcβ3Galβ4GlcNAc

In a preferred embodiment at least one α3-sialyl oligosaccharide sequence according to the formula are used. In a preferred embodiment the oligosaccharide sequences are used in combination with an oligosaccharide containing at least one α6-linked oligosaccharide sequence.

More preferably the present invention is directed to divalent α6-linked oligosaccharide sequences SAα6Galβ4[Glc(NAc)₀ₒᵣ₁)]₀ₒᵣ₁{β3Gal[β4Glc(NAc)₀ₒᵣ₁]₀ₒᵣ₁}₀ₒᵣ₁. SA may be a natural or synthetic sialic acid analogue or derivative capable of replacing Neu5Ac in on or both of the sialic acid binding sites according to the invention.

### Preferred Spacer Structures

The spacer may comprise 2-4 N-acetylactosamine units and a galactose residue as in saccharide 21, in a poly-N-acetylactosamine type substance according to the invention. Alternatively the spacer may comprise a flexible divalent spacer such as the “DADA” molecules according to the invention. The present invention is further directed to the use of any flexible organic, non-carbohydrate spacer of desired length and suitable for cross-linking the two oligosaccharide.

The flexible spacers preferably contain flexible alkyl-structures with at least one, more preferably 2 and even more preferably 3-CH₂— units. In a preferred embodiment rigidity is added to the spaced by one and in another preferred embodiment by two amide bonds. In a preferred embodiment the spacer is linked to the oligosaccharide sequences by an aldehyde reactive structure, preferably by an oxime-bond formed from an amino-oxyterminal structure, in preferred embodiment one aldehyde reactive structure is used and more preferably two aldehyde reactive structures are used. The use of aldehyde reactive structures makes conjugation of the carbohydrate most effective.

SAα3Gal-containing poly-N-acetyllactosamines and special spacer modified conjugates The present invention therefore directed to divalent alpha3-sialylated structures according to the formula SAα3Galβ4[Glc(NAc)₀ₒᵣ₁)]₀ₒᵣ₁{(β3Gal[β4Glc(NAc)₀ₒᵣ₁]₀ₒᵣ₁}₀ₒᵣ₁ linked with spacer to form dimeric or oligomeric or polymeric structures having specific distances between two active sialylated terminal structures, wherein SA is sialic acid preferably N-acetylneuraminic acid, Neu5Ac. SA may be a natural or synthetic sialic acid analogue capable of replacing Neu5Ac in on or both of the sialic acid binding sites according to the invention. The spacer may comprise 2-4 N-acetylactosamine units and a galalactose residue as in saccharide 20. Alternatively the spacer may comprise a divalent spacer such as the “DADA” molecules according to the invention. In a specific embodiment the present invention is directed to divalent poly-N-acetylactosamine type structures such as the oligosaccharide 20 others shown to be active by hemagglutinin inhibition and 9, which is active especially when presented in polyvalent form on a solid phase.

The distance of the between the sialic acid residues from carboxylic acid group to carboxylic acid spacer linked conjugates or the poly-N-acetyllactosamine conjugates is preferably about 27 Angstrom or more. The 27 Å is the distance in the complex structure of hemagglutinin and the saccharide 7, In another embodiment the terminal oligosaccharides are linked with a spacer structure so that the distance between the terminal oligosaccharide structures is between 27 and 54 Å so that the divalent structure does not easily reach to two primary sites on hemagglutinin trimer.

The invention is further directed to divalent conjugates according to the invention when the distance between the sialic acid residues is about 55 Å or more.

### The Large Polylactosamine Epitopes: High Affinity Ligands for Influenza Virus

The present invention is directed to a high affinity ligands for hemagglutinin protein of influenza virus. The inventors have further found out that the influenza virus hemagglutinin bind complex human glycans such as poly-N-acetyllactosamine type carbohydrates using a large binding site according to the invention on its surface. The present invention is especially directed to special large poly-N-acetyllactosamine structures with effective binding with the large binding site. The special large poly-N-acetyllactosamines are called here “the large polylactosanine epitopes”.

### The Large Binding Site

Furthermore, the present invention is especially directed to the novel large binding site on surface of hemagglutinin, called here “the large binding site”. The large binding site binds effectively special large polylactosmine type structures and analogs and derivatives thereof with similar binding interactions and/or binding surface in the large binding site.

The large binding site includes:
1. the known primary binding site for sialylated structures in human influenza hemagglutinin, the region of the large binding site is called here “the primary site” or “Region A” and
2. so called secondary sialic acid binding site on the surface of the hemagglutinin, wherein the sialic acid or surprisingly also certain other terminal monosaccharide residues or analogs thereof can be bound by novel binding mode, the region of the large binding site is called here “the secondary site” or “Region C” and
3. a groove-like region on surface of hemagglutinin bridging the primary and to secondary sites, called here “the bridging site” or “Region B”.

### The Conserved Peptide Sequences of the Large Binding Site

Molecular modelling of mutated sites on the surface of influenza hemagglutinin revealed that many of amino acid residues on the large binding site are strongly conserved and part of the amino acid residues are semiconservatively conserved. The conservation of the protein structures further indicates the biological importance of the large binding site of the hemagglutinin. The virus cannot mutate nonconservatively the large binding site without losing its binding to the sialylated saccharide receptors on the target tissue. It clear that the large binding site is of special interest in design of novel medicines for influenza, which can stop the spreading of the virus.

### Conservation of the Large Binding Site Between Species

Furthermore, it was found out that the large binding sites in general are conserved between various influenza virus strains. Mutations were mapped from hemagglutinins from 100 strains closely related to strain X31. The large binding site was devoid of mutations or contained conservatively mutated amino acids in contrast to the surrounding regions. The large binding site recognized sialylated polylactosamines.

Animal hemagglutinins, especially avian hemagglutinins, are important because pandemic influenza strains has been known to have developed from animal hemagglutinins such as hemagglutinins from chicken or ducks. Also pigs are considered to have been involved in development of new influenza strains. The recognition of large carbohydrate structures on the surface of influenza hemagglutinin has allowed the evolution of the large binding site between terminal carbohydrate structures containing α3- and/or α6-linked sialic acids.

The pandemic strains of bird origin may be more α3-sialic acid specific, while the current human binding strains are more α6-specific. The present invention is further directed to mainly or partially α3-specific large binding sites. The present invention is further directed to substances to block the binding to mainly or partially α6-specific large binding sites.

### Design of Vaccines and Antibodies.

The large binding site and its conserved peptide sequences are of special interest in design of novel vaccines against influenza virus. The general problem with vaccines against influenza is that the virus mutates to immunity. A vaccine inducing the production of antibodies specific for the large binding site and its conserved peptide sequences will give general protection against various strains of influenza virus.

Furthermore, the invention is directed to the use of antibodies for blocking binding to the large binding site. Production of specific antibodies and human or humanized antibodies is known in the art. The antibodies, especially human or humanized antibodies, binding to the large binding site, are especially preferred for general treatment of influenza in human and analogously in animal.

Methods for producing peptide vaccines against influenza virus are well-known in the art. The present invention is specifically directed to selecting peptide epitopes for immunization and developing peptide vaccines comprising at least one di- to decapeptide epitope, more preferably at least one tri- to hexapeptide epitope, and even more preferably at least one tri to pentapeptide epitope of the “large binding site” described by the invention in Table 1.

The peptide epitopes are preferably selected to contain the said peptide from among the important binding and/or conserved aminoacids according to the Table 1, more preferably at least one peptide epitope is selected from region B. In another preferred embodiment two peptides are selected for immunization with two peptides so that at least one is from region B and one from region A or B. Preferably the peptide epitope is selected to comprise at least two conserved amino acid residues, in another preferred embodiments the peptide epitope is selected to comprise at least three conserved amino acid residues. In a preferred embodiment peptide epitope is modelled to be well accessible on the surface of the hemagglutinin protein.

### The Complex Structure Between Large Polylactosamine Epitopes and the Large Binding Site

The invention is further directed to a substance including a complex of influenza virus hemagglutinin with a large polylactosamine epitope, called here “the complex structure”. The present invention is especially directed to the use of the complex structure for design of analogous substances with binding affinity towards hemagglutinin of influenza.

### The Specific Binding Interactions.

The present invention is directed to the use of the binding interactions observed between the large polylactosamine epitopes and the large binding site, called here “the specific binding interactions” for design of novel ligands for influenza virus hemagglutinin.

### The Large Polylactosamine Epitopes a High Affinity Ligands for Influenza Virus

The present invention is specifically directed to effectively influenza virus binding polylactosamine structures such as Neu5Acα6Galβ4GlcNAcβ3[Neu5Acα6Galβ4GlcNAcβ3Galβ4GlcNAcβ6]Galβ4Glc and similar structures and analogues.

The present invention is especially directed to the structural analogs according to the formula

SAα3/6Hex(NAc)ₙ₁β4Hex(NAc)ₙ₂β3[Sac13/6Hex(NAc)ₙ₃β4Hex(NAc)ₙ₄β3Hex(NAc)ₙ₅β4Hex(NAc)ₙ₆β6]Hex(NAc)ₙ₇β4Hex(NAc)ₙ₈

wherein
Hex is a hexopyranosylresidue, Gal or Glc
SA is sialic acid or analog or derivative thereof, preferably Neu5Ac:

### Sac1 is Hex(NAc)ₙ₉β or SAα

n1, n2, n3, n4, n5, n6, n7, n8 and n9 are integers either 0 or 1

SAα6Galβ4Glc(NAc)ₙ₂β3[Sac13/6Galβ4GlcNAcβ3Galβ4Glc(NAc)ₙ₆β6]Galβ4Glc(NAc)ₙ₈

wherein SA is sialic acid or analog or derivative thereof, preferably Neu5Ac:

### Sac1 is Glc(NAc)ₙ₉β or SAα.

The tolerance of modifications is studied using molecular modelling as described by the invention. The Hexβ4 structures are preferably Galβ4, and other Hex units Glc, in a preferred embodiment one of n8, n6 or n2 is 0, more preferably n6 is 0 or n8 is 0 and most preferably n6 is 0. In general structures with 1-3 differences from the preferred structures are preferred, more preferably with 2 differences and most preferably with one difference.

The preferred poly-N-acetyllactosamine structures may be represented as following divalent sialosides with specific carbohydrate spacer structures:

A poly-N-acetyllactosamine sialoside when the spacer according to the invention is

R₁β3/6{R₂β3Hex(NAc)ₙ₅β4Hex(NAc)ₙ₆β6/3}Hex(NAc)ₙ₇β4[Hex(NAc)ₙ₈]ₙ₉

wherein Hex is a hexopyranosylresidue, Gal or Glc; { } represent a branch in the structure, R1 and R2 are sialyl-oligosaccharide sequences according to the invention preferably trisaccharides or a trisaccharide and a pentasaccharide, the penta saccharide preferably being linked to the branched Hex.

n5, n6, n7, n8 and n9 are integers either 0 or 1;

and more preferably as

A poly-N-acetyllactosamine sialoside when the spacer according to the invention is

R₁β3/6[R₂β3Galβ4GlcNAcβ6/3]Gal(β4Glc)ₙ₉

wherein
R1 and R2 are sialyl-oligosaccharide sequences according to the invention preferably trisaccharides or a trisaccharide and a pentasaccharide, the penta saccharide preferably being linked to the branched Gal, and 9 is an integer either 0 or 1;

Even more preferably the invention is directed to

R₁β3[R₂β3Galβ4GlcNAcβ6]Gal(β4Glc)ₙ₉

Wherein R1 and R2 are sialyl-trisaccharide sequences according to the invention n9 is an integer either 0 or 1;
Branch Specific poly-N-acetyllactosamine Library for Screening Biological (Lectin) Binding

It was found out for the first time in the present invention that branch specific poly-N-acetyllactosamine library is an effective tool for screening biological binding, especially binding of specific poly-N-acetyllactosamines by lectins (carbohydrate binding proteins) such as hemagglutinin protein of viruses.

The preferred library may also comprise disialyl-oligosaccharide compounds containing flexible spacer as described by the invention, in a preferred embodiment the poly-N-acetyllactosamine library contains both branch specific oligosaccharides and disialyl-oligosaccharide compounds containing flexible spacer.

In a preferred embodiment the present invention is directed to the use of the oligosaccharide library for screening of binding specificities according to the invention. Preferably the library for screening specificities of viruses, especially influenza virus, contains some or all of the preferred substances according to the present invention.

The present invention is especially directed to the use of the branch specific poly-N-acetyllactosamine library for the screening of binding specificities of animal lectins or animal poly-N-acetyllactosamine binding lectins and more preferably human specificities of human lectins or human poly-N-acetyllactosamine binding lectins. The branch specific poly-N-acetyllactosamine library according to the invention indicates specific collection of defined polylactosamine structures which are usually isomers with the same molecular weight. In the prior art symmetric polylactosamine libraries or collections with similar branches have been used for screening various bioactivities including selectin ligands or receptor involved the fertilization of mouse. In contrast to previous works the present invention realizes the usefulness and special recognition of branched poly-N-acetyllactosamines with different terminal structures.

The prior art also describes synthesis of branch isomer structures, without specific biological indications, and in some cases separation of these by complicated chromatographic methods. The methods according to the present invention allow separation of the branched poly-N-acetyllactosamines by simple ion exchange or other known methods.

Special Synthesis Methods for the Branched poly-N-acetyllactosamines

The inventor further discovered that it is possible to synthesize essentially pure branch specific poly-N-acetyllactosamines by enzymatic synthesis. This has advantage as a simple method for example in contrast to traditional synthesis methods by organic chemistry using several protecting and deprotecting steps per monosaccharide residue. The present invention is specifically directed to construction of branch specific poly-N-acetyllactosamines comprising at least two isomeric branches with different lengths using branch specific starting materials. The branch specific starting materials includes
1. LNH, lacto-N-hexaose, Galβ3GlcNAcβ3[Galβ4GlcNAcβ6]Galβ4Glc,
2. an asymmetric pentasaccharide GlcNAcβ3[Galβ4GlcNAcβ6]Galβ4Glc, preferably synthesized by β3-galactosidase reaction from LNH, and
3. branch specifically sialylated structure Neu5Acα6Galβ4GlcNAcβ3[Galβ4GlcNAcβ6]Galβ4Glc and its synthesis by branch specific a6-sialyltransferase reaction by soluble branch specific α6-sialyltransferase.
   Other Specific Synthesis Steps for the Branched poly-N-acetyllactosamine Library

The present invention is further directed to specific synthesis steps including

A) β3-N-acetylglucosaminyltrasterase reaction to sialylated branched poly-N-acetyllactosamine (for example GnT3-reaction in Scheme 1 and GnT3 reactions in Schemes 2, 3 or 4). Sialylated branched poly-N-acetyllactosamine is in preferred embodiments α3-sialylylated on a specific branch or α6-sialylylated on a specific branch. The specific branch is in a preferred embodiment β6-linked branch and in another embodiment β3-linked branch. The preferred β3-N-acetylglucosaminyltrasterase reactions includes reactions by mammalian β3-N-acetylglucosaminyltrasterases most preferably β3-N-acetylglucosaminyltrasterase(s) of human serum.
B) The present invention is further directed to specific sialyltransferase reactions to poly-N-acetyllactosamine containing terminal GlcNAcβ3-structure on a specific branch (for example SAT6 reaction in scheme 2 or SAT3 reaction in Scheme 4). The specific branch is in a preferred embodiment β6-linked branch and in another preferred embodiment β3-linked branch. In a preferred embodiment the sialyltransferase reaction is α6-sialyltransferase reaction and in another preferred embodiment the sialyltransferase reaction is α3-sialyltransferase.
C) The present invention is further directed to use a specifically removable terminal monosaccharide unit as a temporary blocking group on a branched polylactosamine structure. For example in Scheme 4 α6-linked Neu5Ac residue was used as a temporary blocking group. Preferred temporary blocking groups includes hexoses, N-acetylhexosamines, hexosamines, uronic acids or pentoses with following properties
1. the monosaccharide residues transferable to terminal N-acetyllactosamines by specific transferases and
2. removable specific glycosidases
3. the monosaccharide unit is not interfering the synthesis in the other branch (for example it is not acceptor/substrate or inhibitor for another transferase aimed to modify the other branch on the poly-N-acetyllactosamine and it blocks the transfer by the other transferase to the branch it modifies.
More preferably the terminal blocking is Neu5Acα3, NeuNAcα6 and Galα3 and even more preferably Neu5Acα3 or NeuNAcα6 and most preferably Neu5Acα6. It is further realized that numerous terminal monosaccharide units Sialic acid can be specifically removed by sialidases or mild acid treatment which do not affect the backbone poly-N-acetyllactosamine structures. The Galα3 structure can be synthesized by Galα3-transferase and released by alpha-galactosidases.

The inventors do not know similar reaction in the prior art. In general it is considered that the N-acetyllactosamine structures are biosynthetically first galactosylated and then sialylated, in biological synthesis the sialyltransferases may be located later in Golgi complex than galactosyltransferases and especially GölcNAc transferases.

The present invention is specifically directed to construction of library of branched poly-N-acetyllactosamines comprising following structures:

(T1)ₚ₁Galβ4GlcNAc(β3Galβ4GlcNAc)ₙ₁β3[(T2)ₚ₂Galβ4GlcNAc(β3Galβ4GlcNAc)ₙ₂β6]Gal{β4Glc(NAc)ₙ₃}ₙ₄{R}ₙ₅

wherein
[ ] indicates branch in the structure, and { } and ( ) indicates structures optionally present, n1, n2, n3, n4, n5, p1 and p2 are integers 0 or 1,
T1 and T2 are terminal monosaccharide residues with the provision that library contains all possible structures with all values of n1 and n2 and/or
T1 and T2, T1 and T2 are different or differently linked monosaccharide residues so that T1 is either M1 or M2 and T1 is either M1 or M2 and the library contains all variants with different terminal monosaccharide units M1 and M2. Preferably T1 and T2 are Neu5Acα3, NeuNAcα6, Galα3 or GlcNAcβ3.

In a preferred embodiment all possible structures with all values of n1 and n2 and T1 and T2, and even more preferably p1 and p2 are 1.

The present invention is also directed to a library of branched poly-N-acetyllactosamines comprising the following structure:

(T1)ₚ₁Galβ4GlcNAc(β3Galβ4GlcNAc)ₙ₁β3[(T2)ₚ₂Galβ4GlcNAc(β3Galβ4GlcNAc)ₙ₂β6]Gal{β4Glc(NAc)ₙ₃}ₙ₄{R}ₙ₅

wherein
[ ] indicates branch in the structure, and { } and ( ) indicates structures optionally present, n1, n2, n3, n4, n5, p1 and p2 are independently integers 0 or 1,
T1 and T2 are independently terminal monosaccharide residues Fuc, Gal, GlcNAc, NeuNAc or Neu5Ac.

Preferably, said library comprises several branched poly-N-lactosamine structures, T1 being independently in each of the structure Fuc, Gal, GlcNAc, NeuNAc or Neu5Ac.

More preferably, T1 and T2 are independently Neu5Acα3, NeuNAcα6, Galα3 or GlcNAcβ3.

It was further found out that the biantennary oligosaccharide with two lactosamine in the branches, structure 21, Table 3, was very active. The present invention is further directed to the two alpha6-sialic acid comprising poly-N-acetylactosamines according to the formula:

SAα6Galβ4Glc(NAc)ₙ₂β3{Galβ4Glc(NAc)ₙ₄β3}ₙ₃[Sac13/6Galβ4GlcNAcβ3Galβ4Glc(NAc)ₙ₆β6]Galβ4Glc(NAc)ₙ₈,

Wherein the SA is sialic acid or analog or derivative thereof, preferably Neu5Ac:
Sac1 is Glc(NAc)ₙ₉β or SAα. And n2, n3, n4, n6 and n8 are independently 0 or 1.

The present invention is further directed to the analogs of the disialylated structures wherein the terminal oligosaccharides are connected with a spacer. In a preferred embodiment the spacer structure can be conjugated directly to the reducing end of a sialyl oligosaccharide without protecting the oligosaccharides or with a protecting group only on the carboxylic acid group of the sialic acids, alternatively preferably sialic acid can be transferred to saccharide after the conjugation of the spacer. The present invention shows a specific aldehyde reactive conjugation to divalent aminooxy-structure containing spacer. A preferred spacer structure is a divalent aldehyde reactive spacer with similar number of atoms than the “DADA” spacer shown in the examples. Preferably the similar number of atoms is within 3 atoms in the length of the spacer.

The invention showed that the binding of the influenza virus to the natural large poly-N-acetyllactosamines to the large binding site of the hemagglutinin could be inhibited by specific oligosaccharides. The present invention is directed to assay to be used for screening of substances binding to the large binding site. Preferably the assay comprises the large binding site, a carbohydrate conjugate or poly-N-acetyllactosamine ligand binding to the large binding site according to the invention and substances to be screened. The substances to be screened are screened for their ability to inhibit the binding between the large binding site and the saccharide according to the invention. The assay may be performed in solution by physical determination such as NMR-methods or fluorescence polarization, by labelling one of the compounds and using various solid phase assay wherein a non-labelled compound is immobilized on a solid phase and binding of a labelled compound is inhibited for example. The substances to be screened may be libraries of chemical synthesis, peptides, nucleotides, aptamers, antibodies etc.

### In Silico Screening

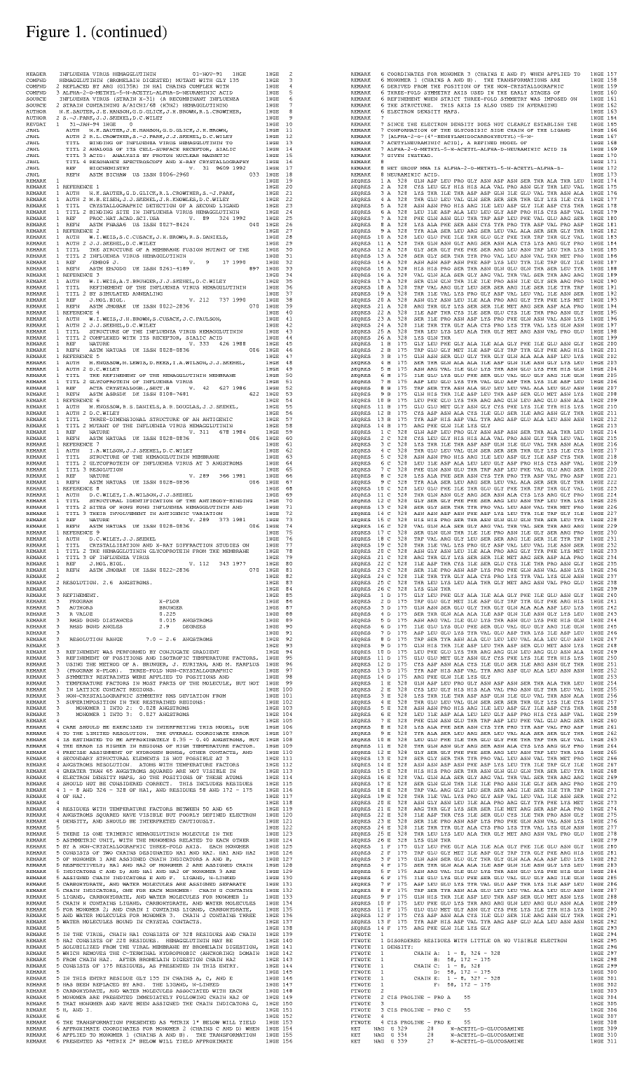

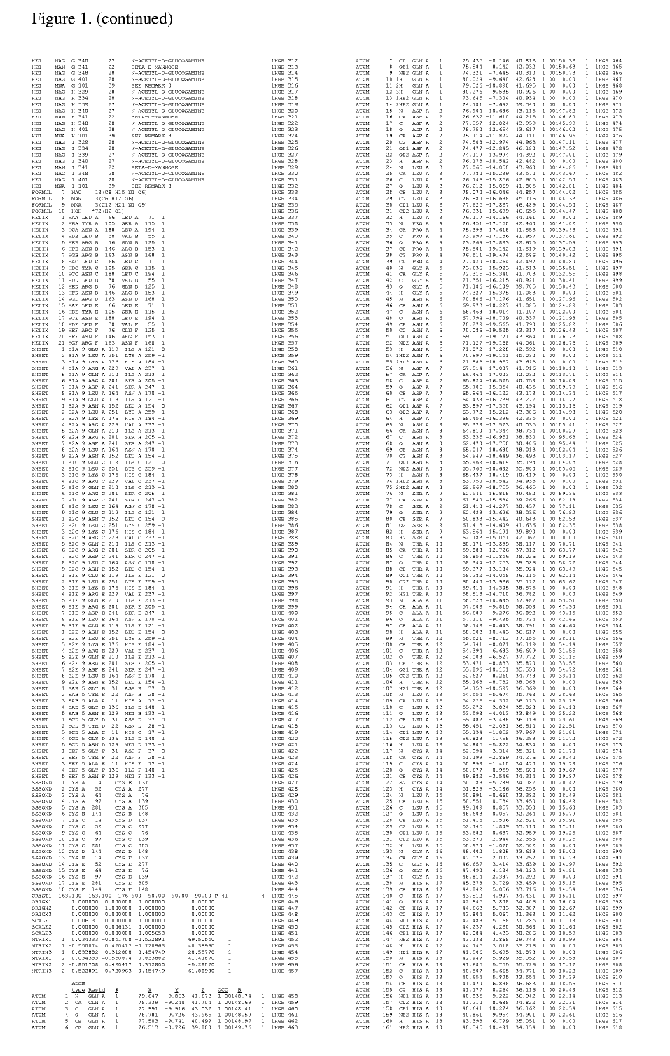

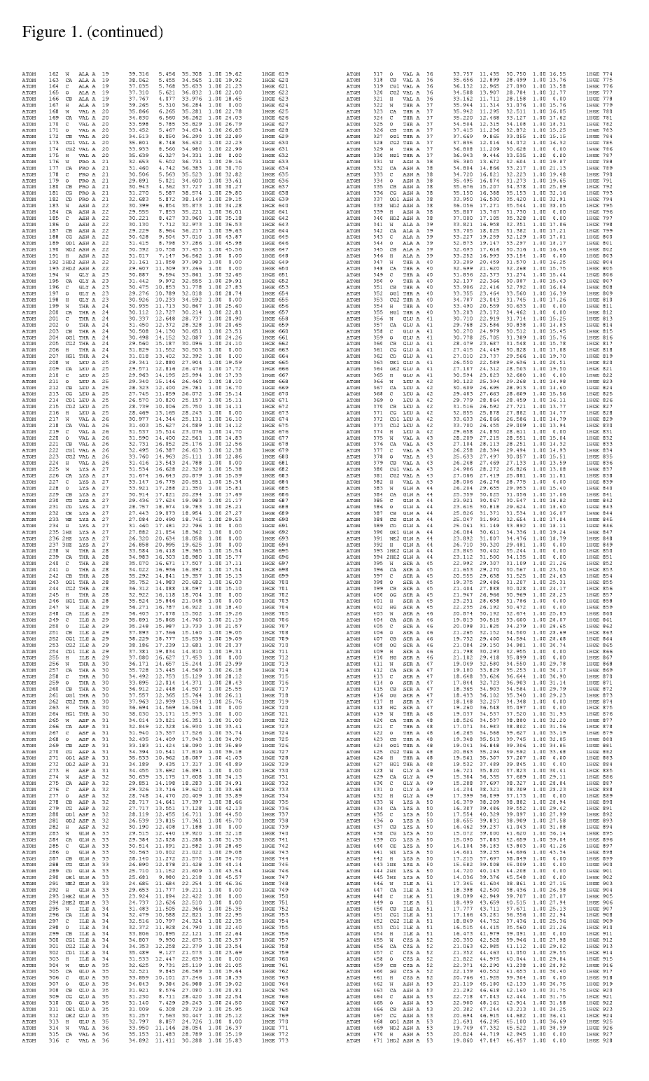

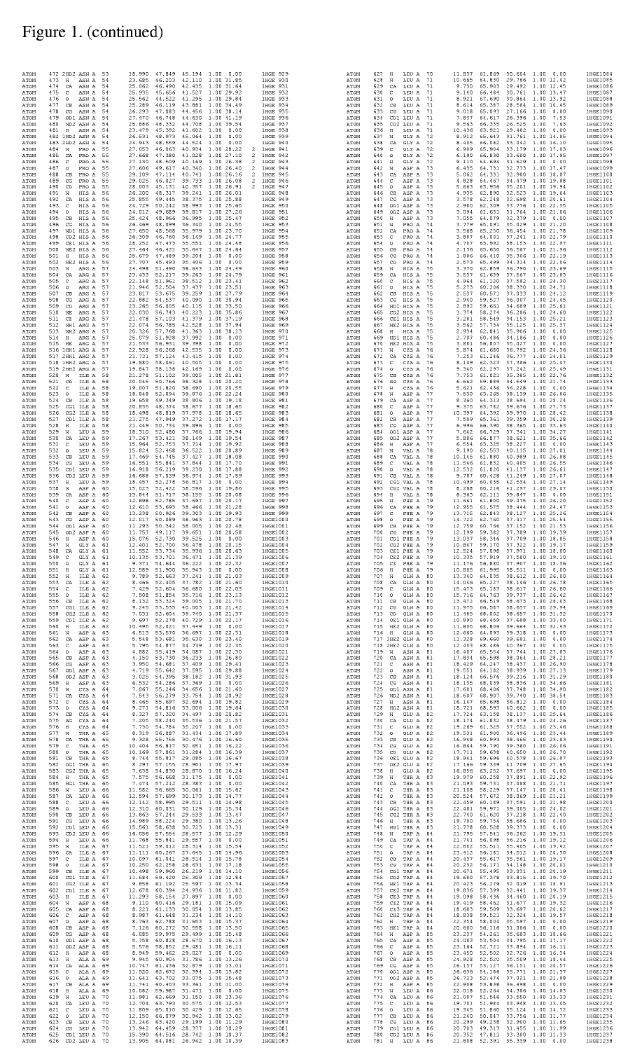

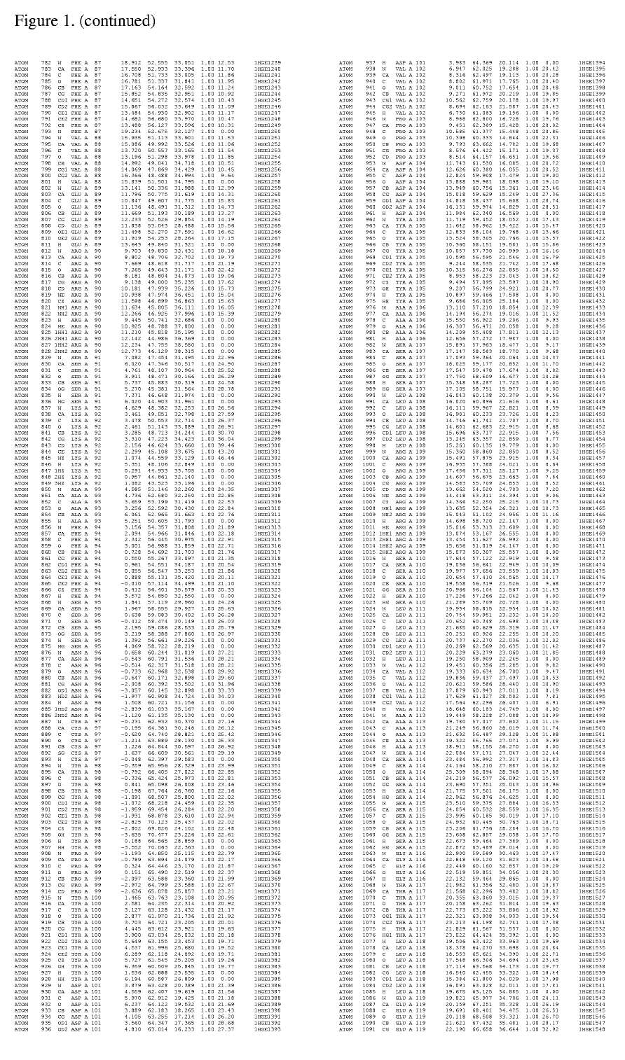

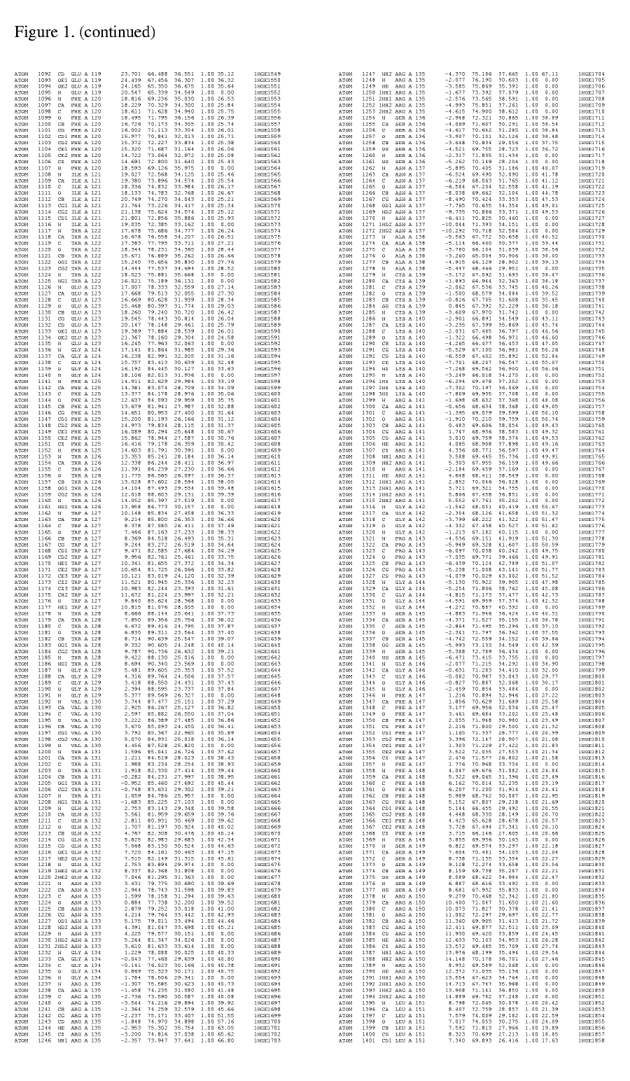

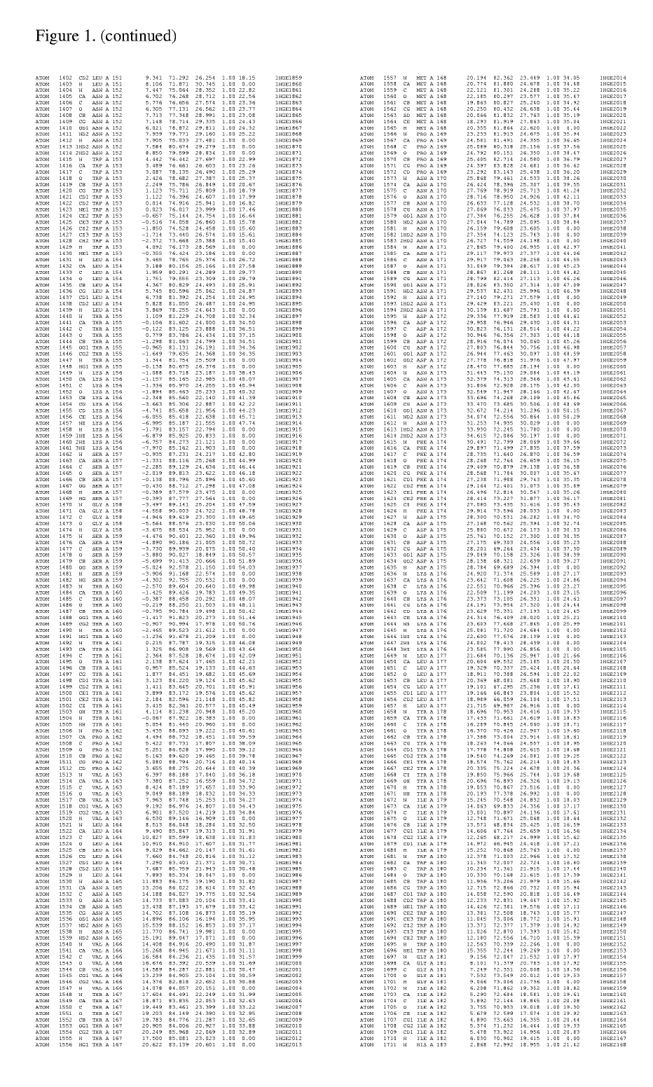

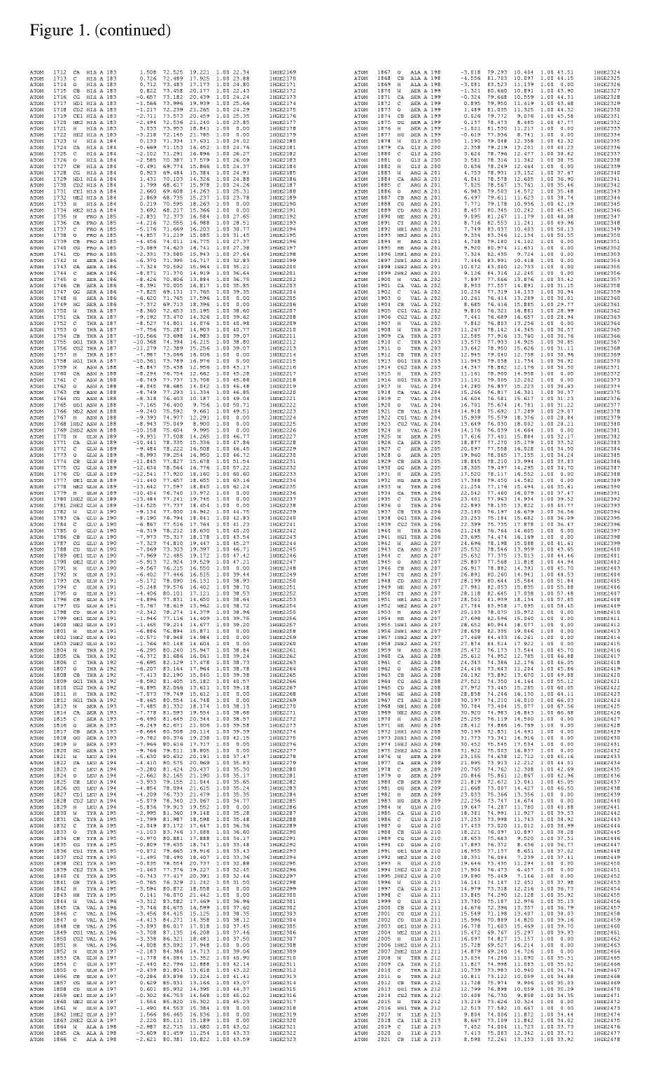

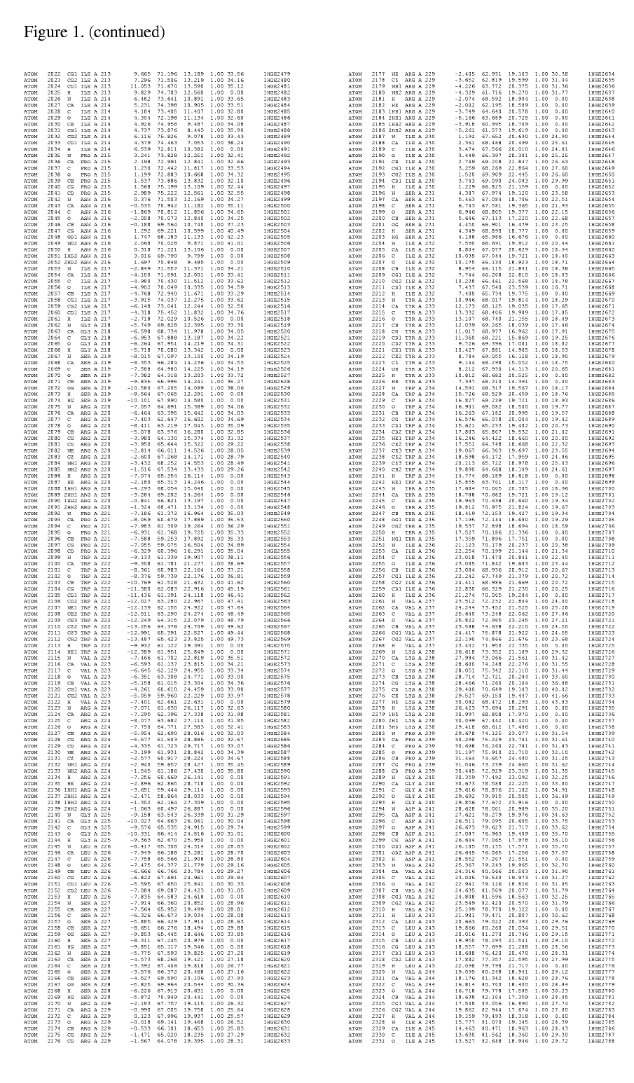

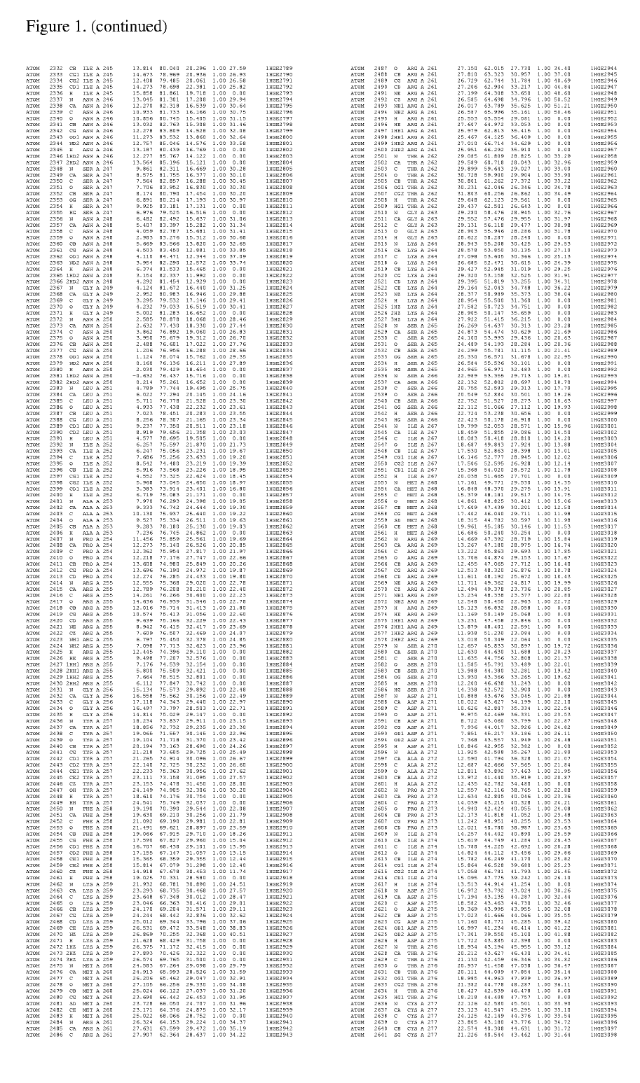

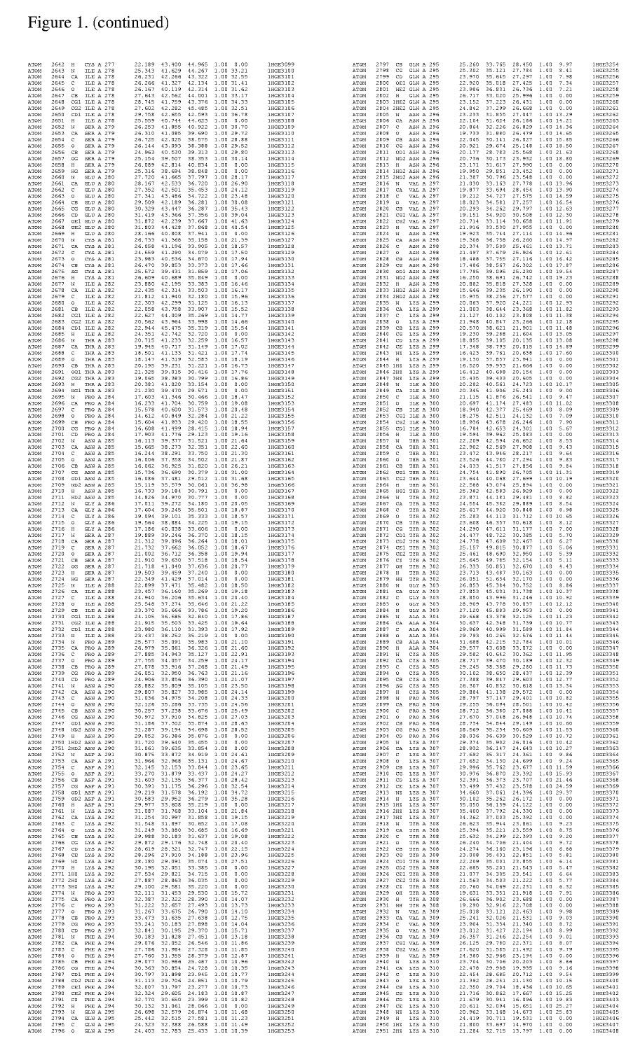

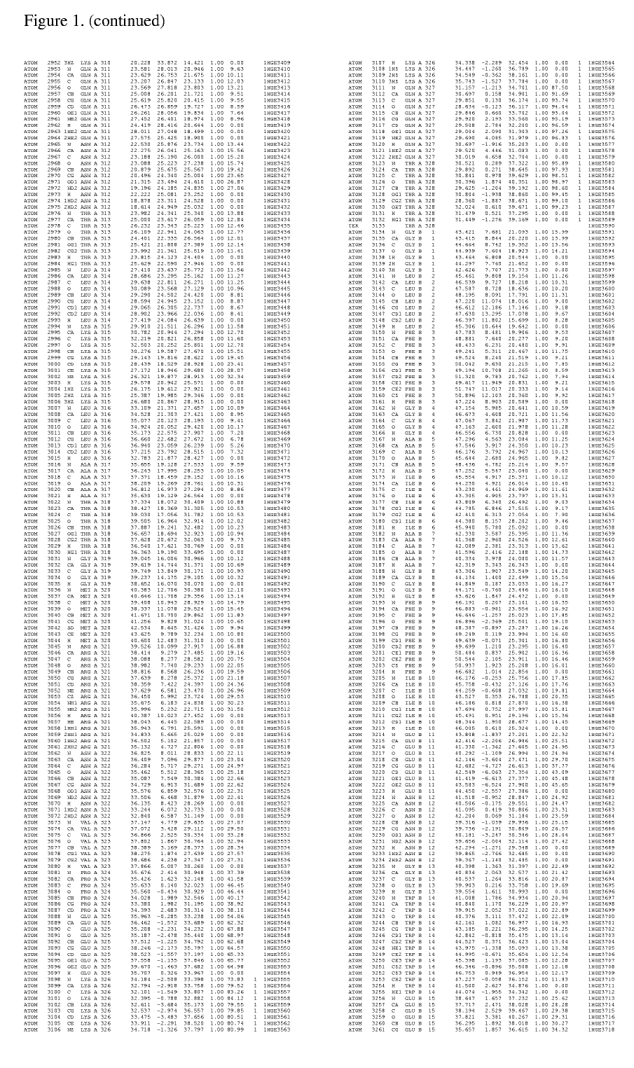

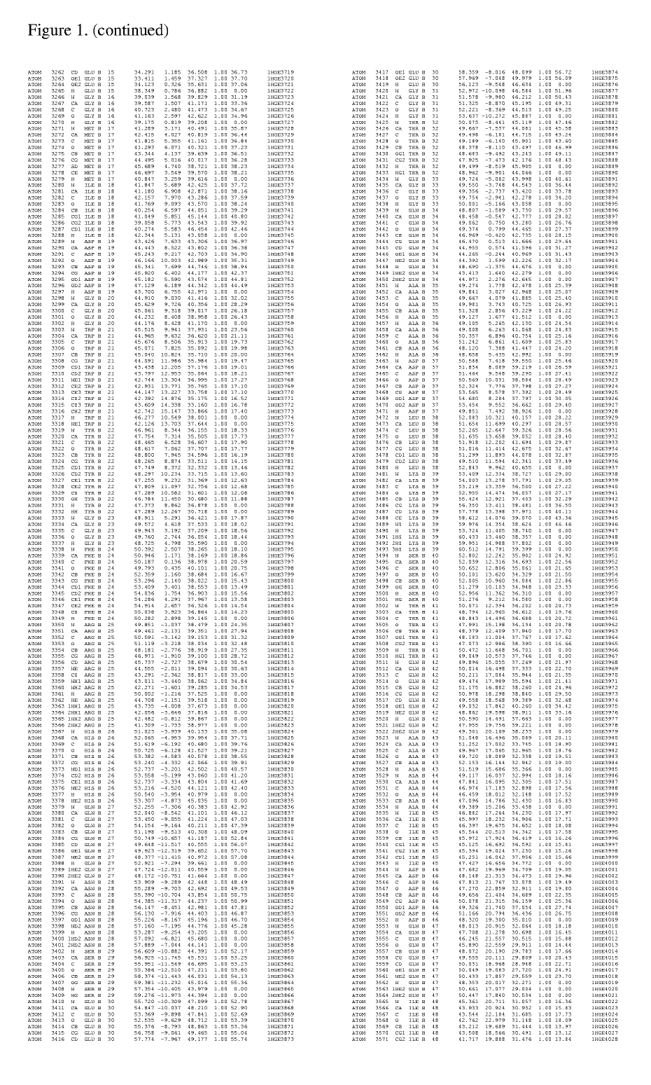

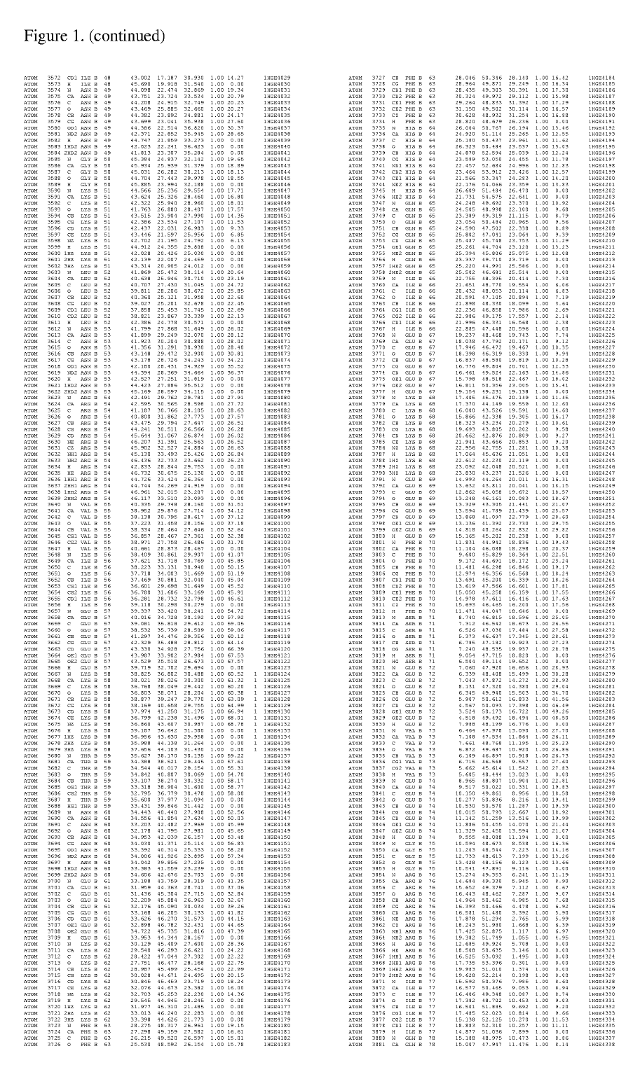

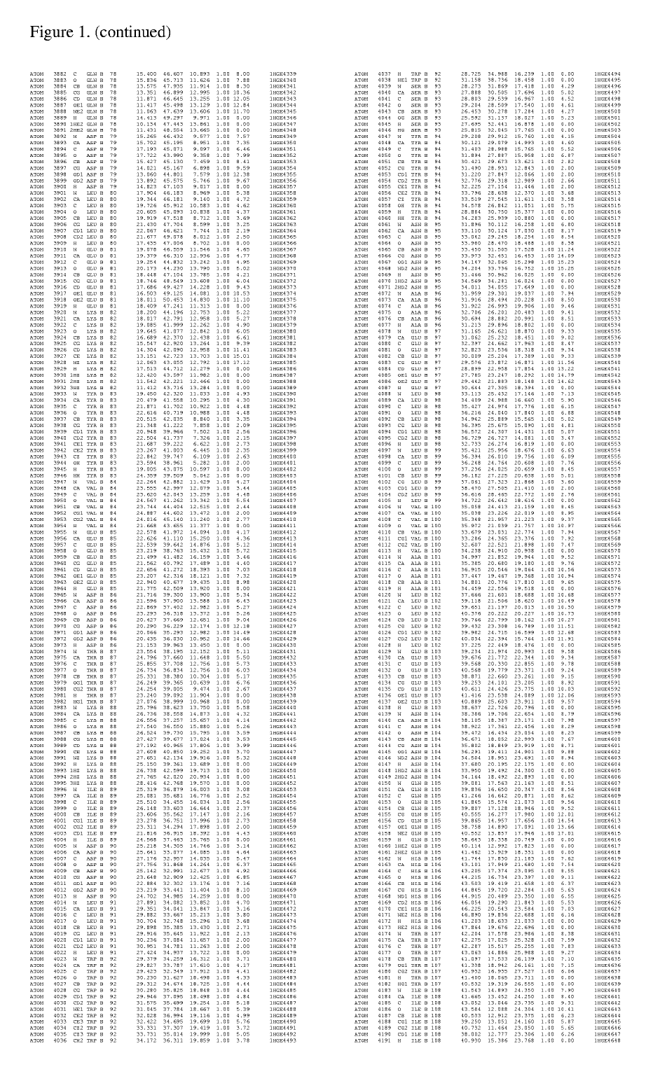

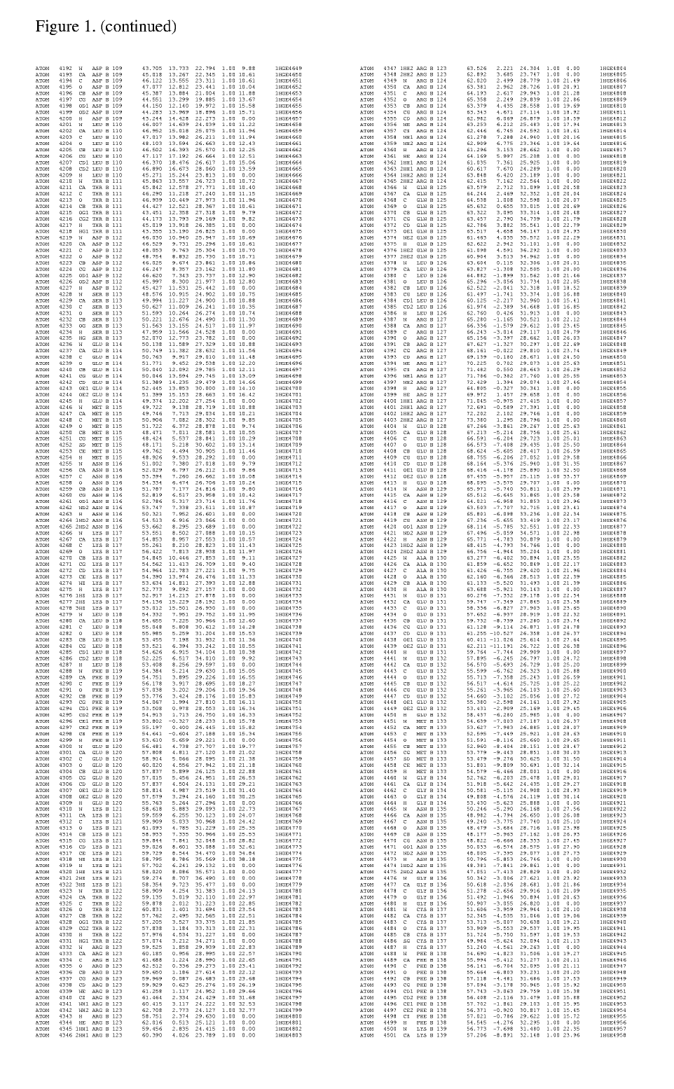

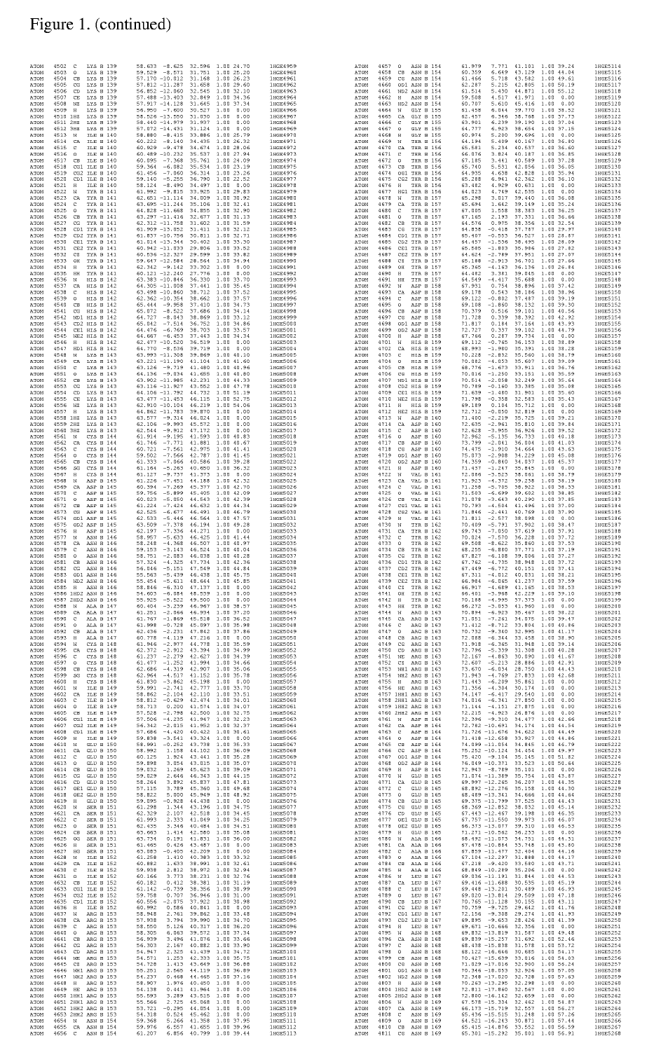

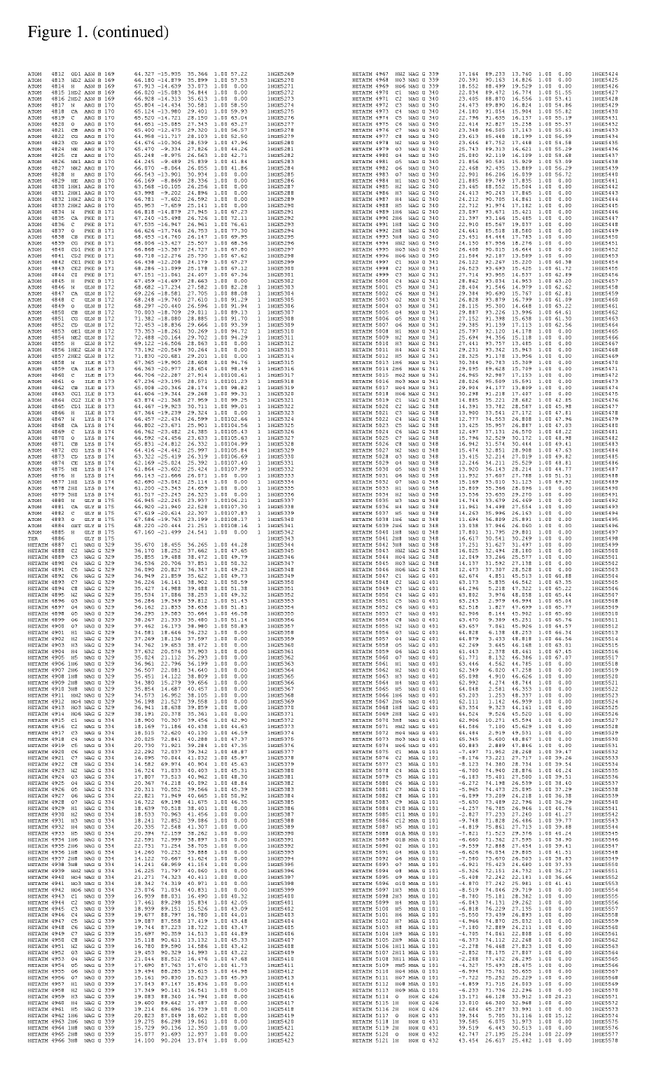

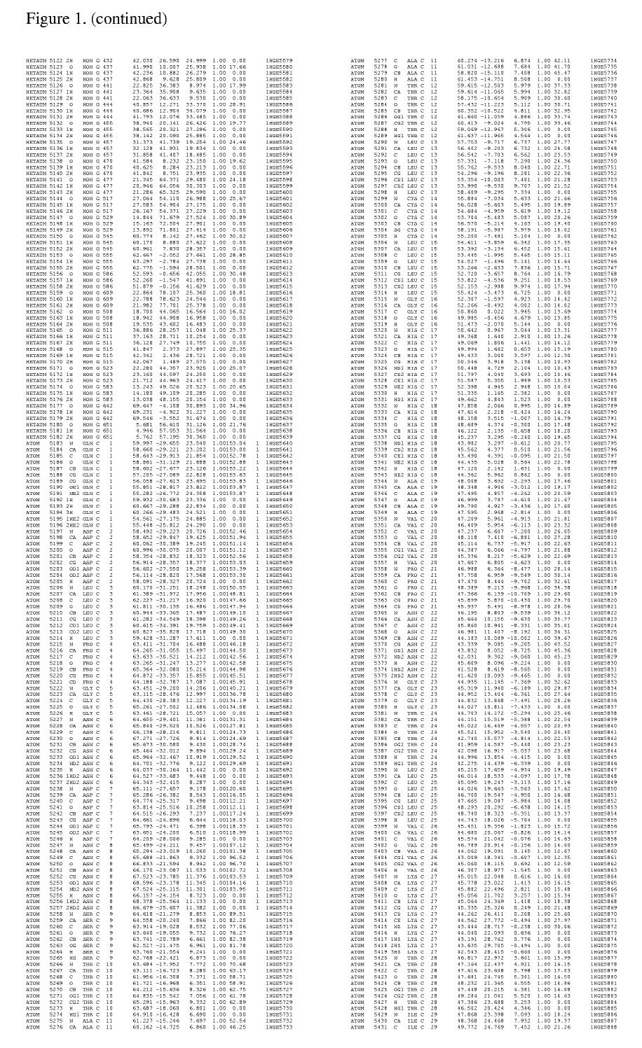

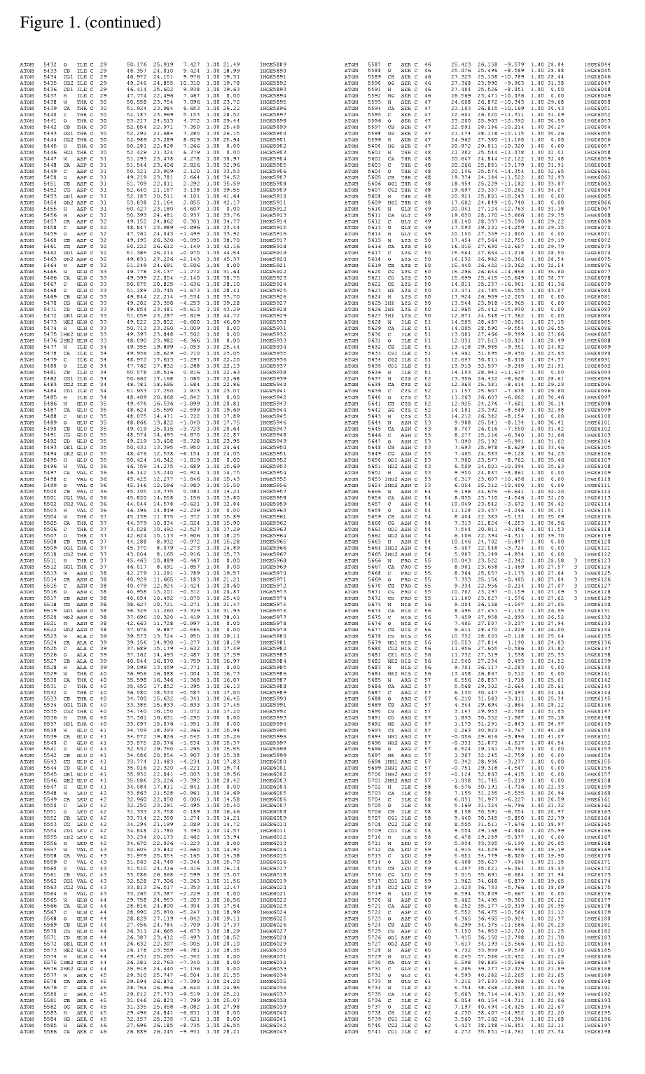

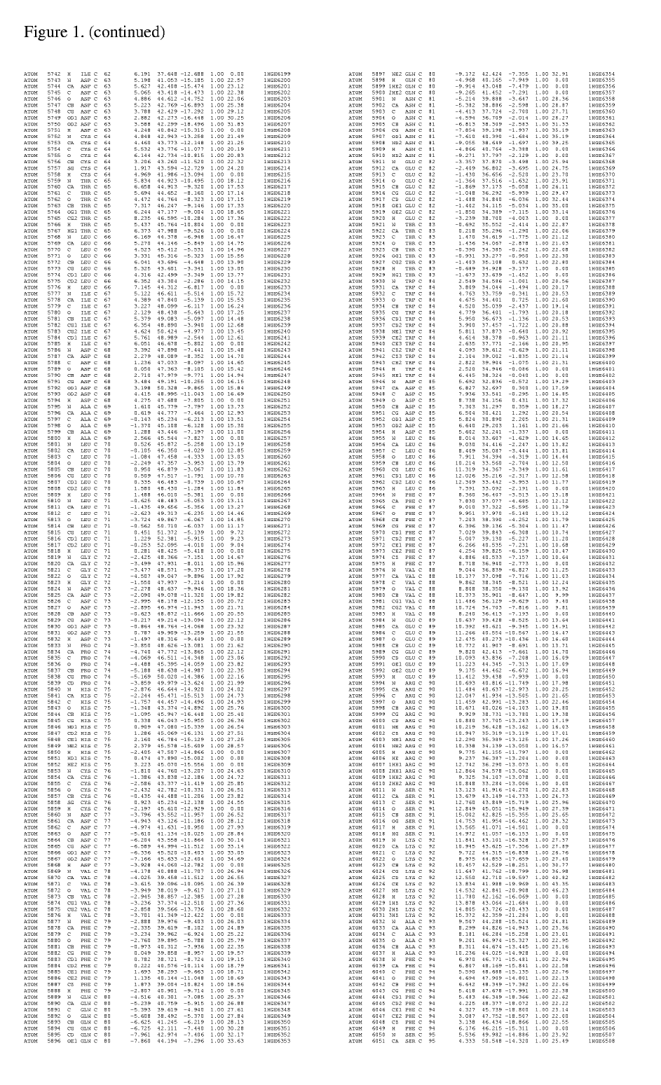

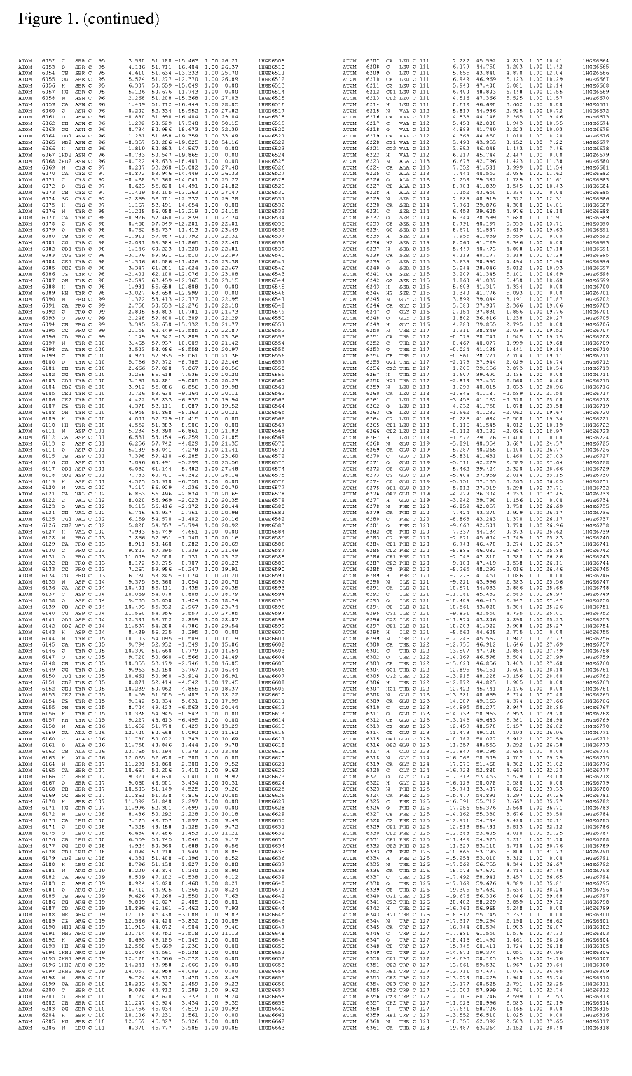

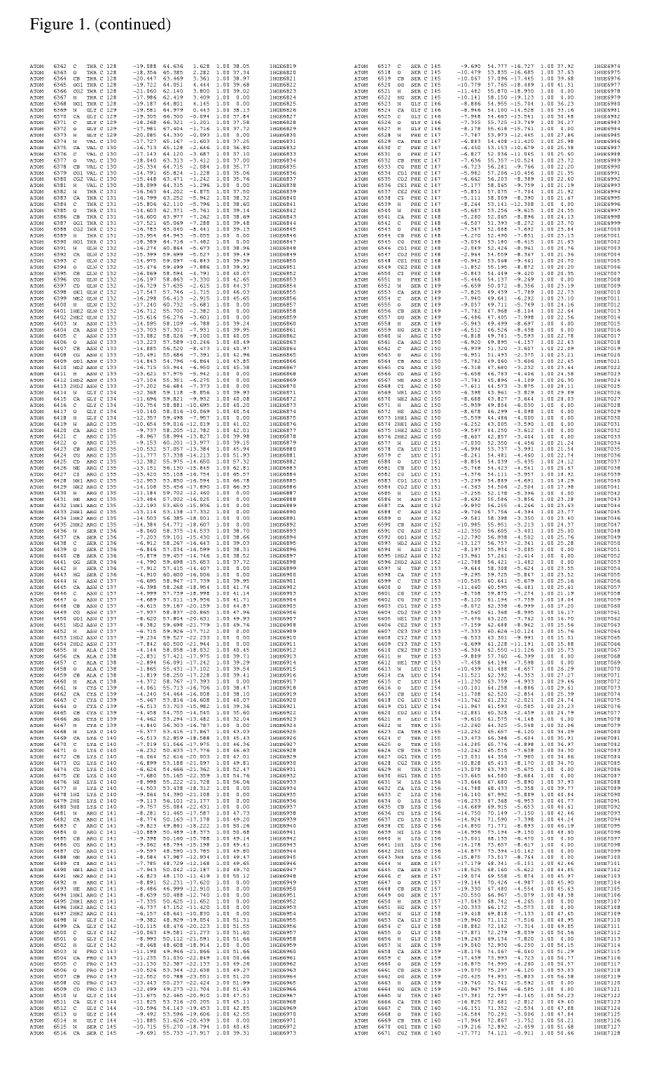

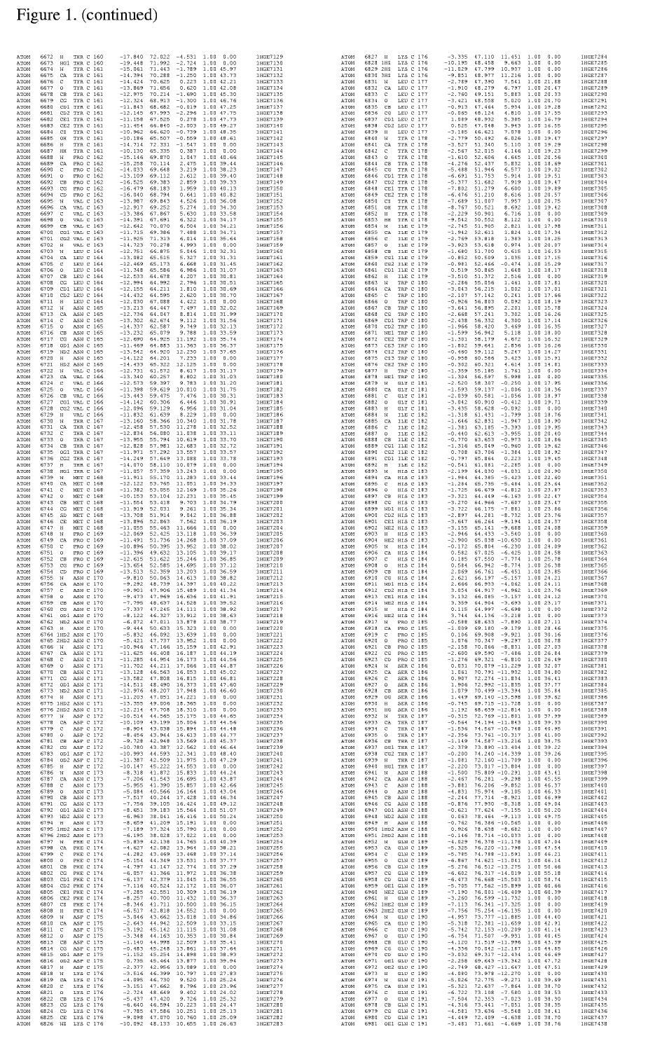

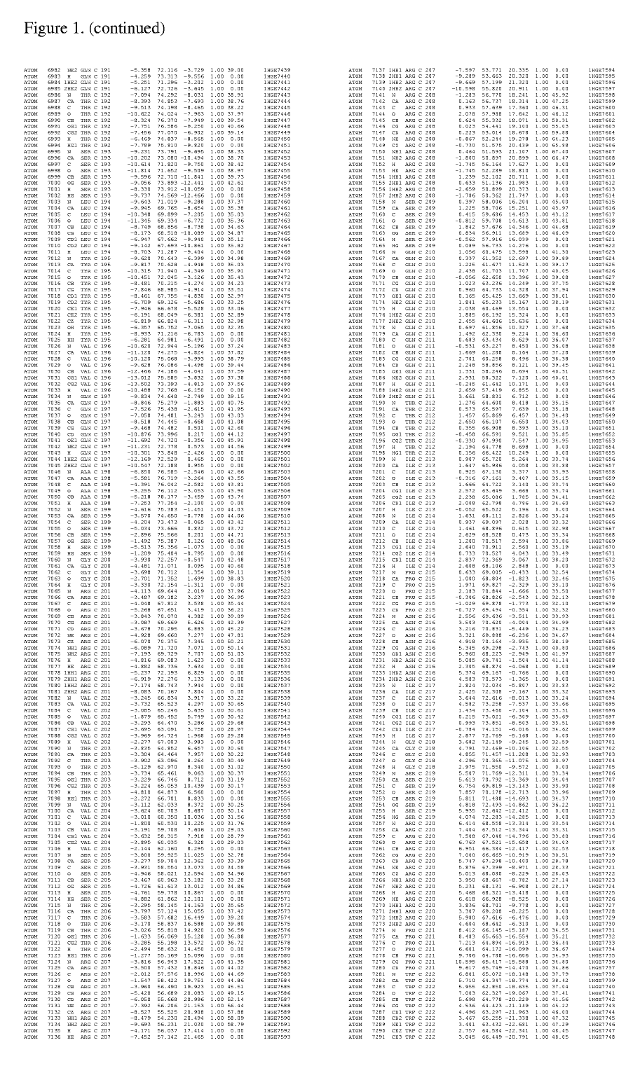

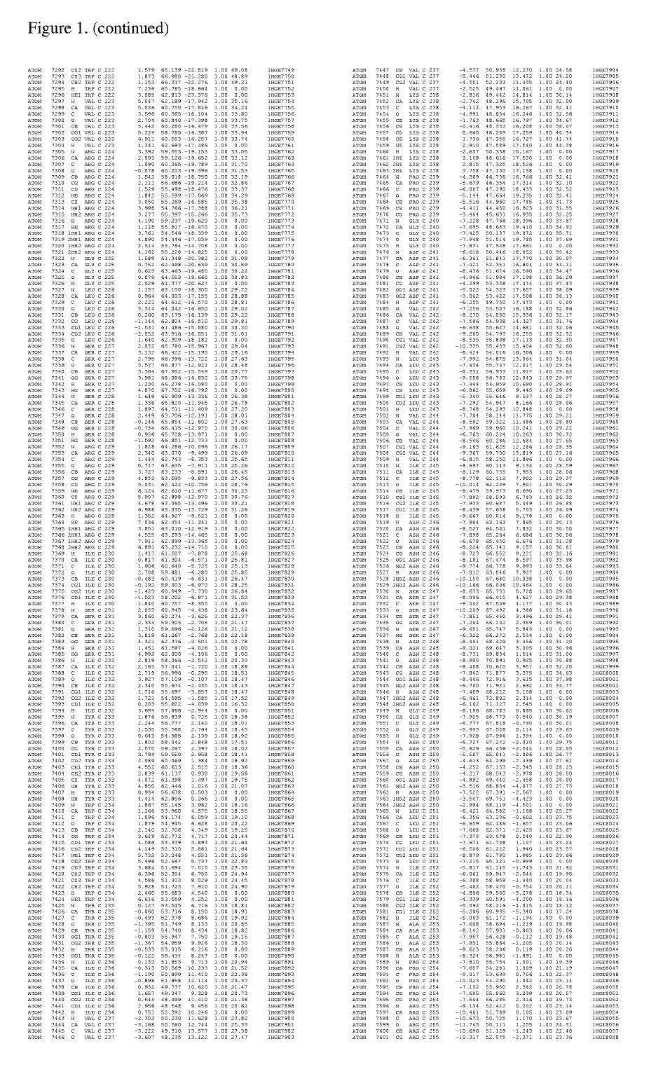

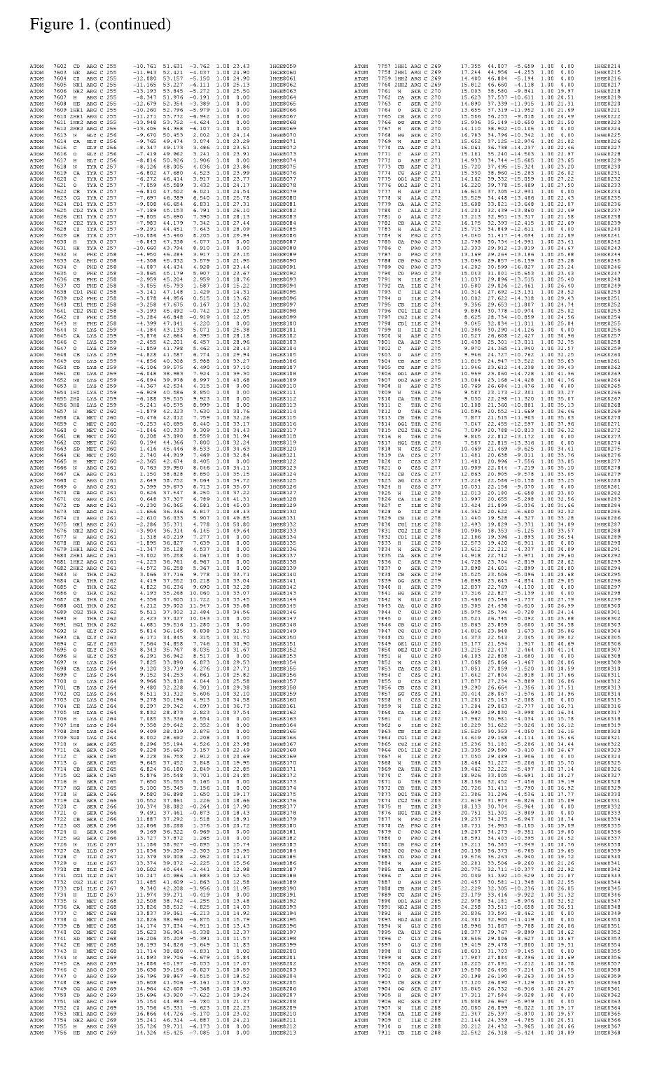

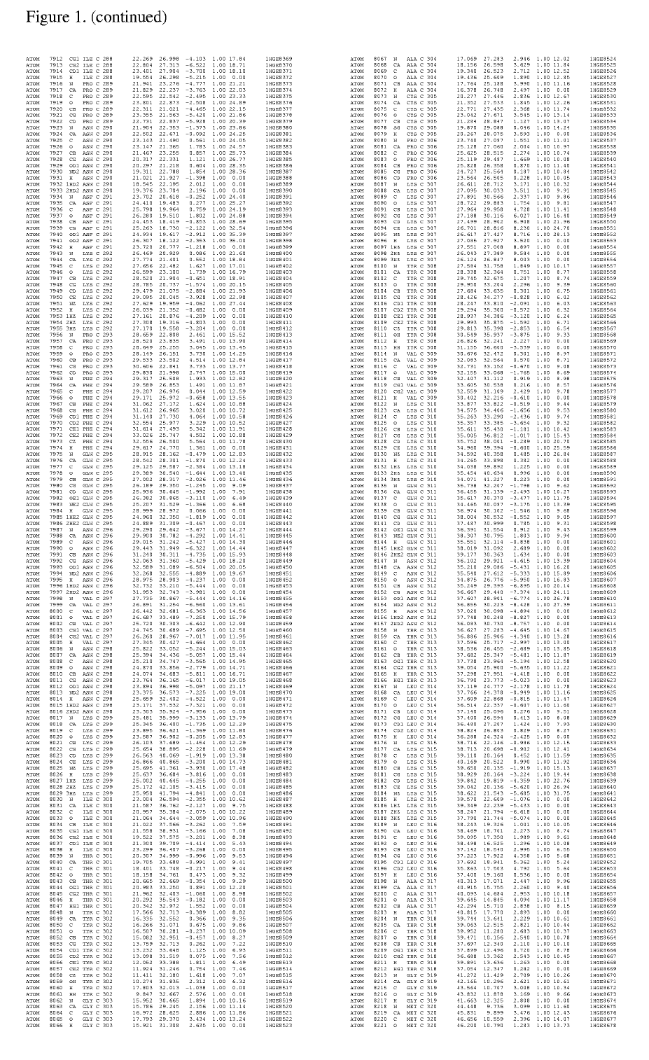

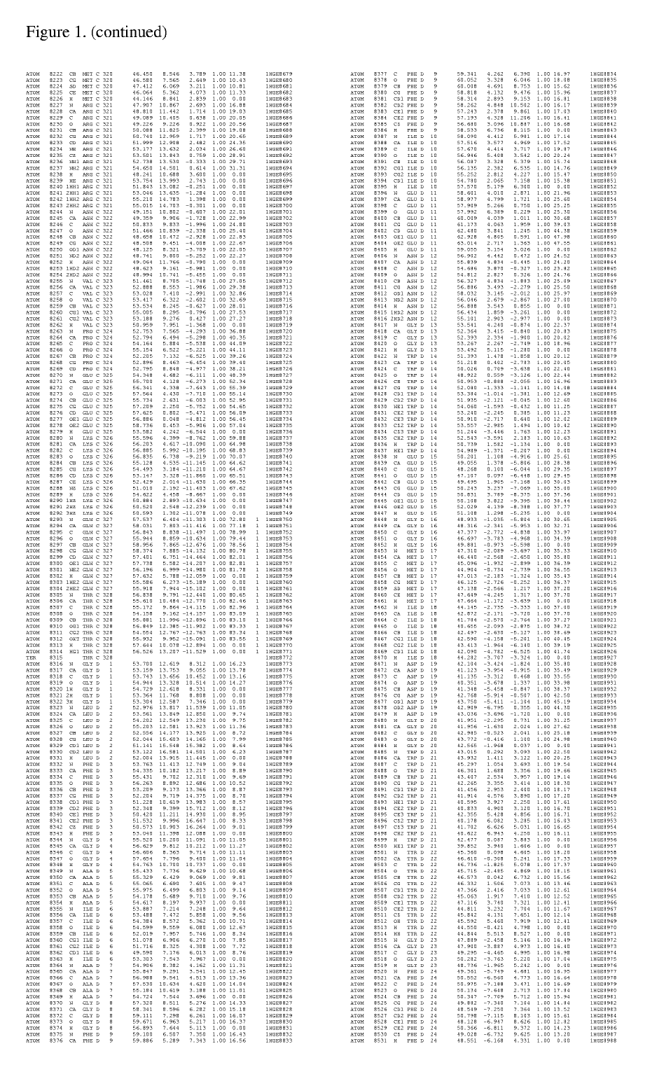

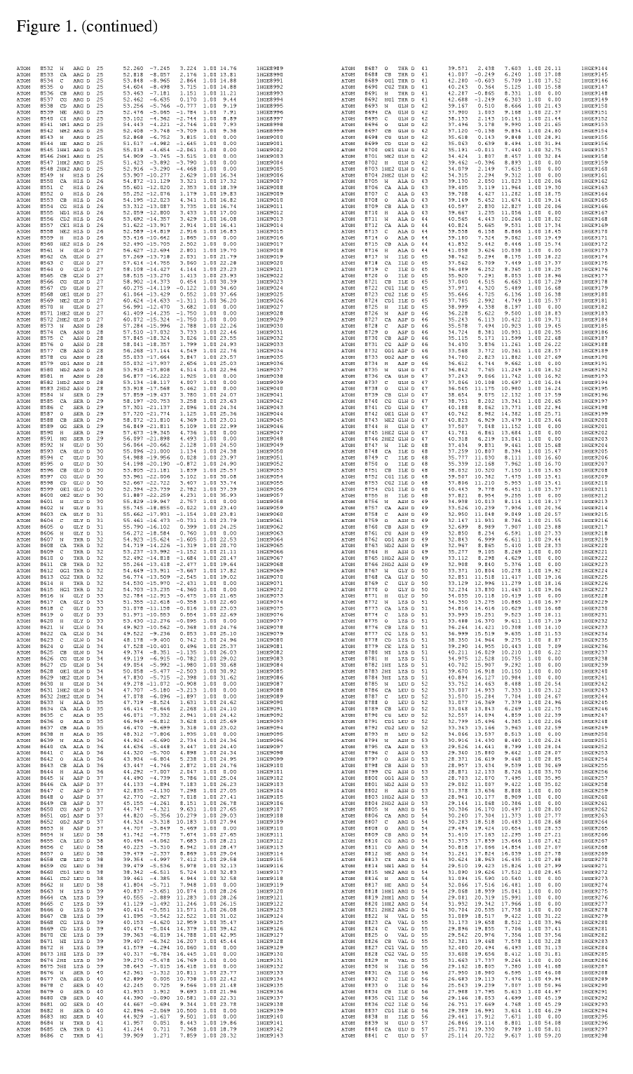

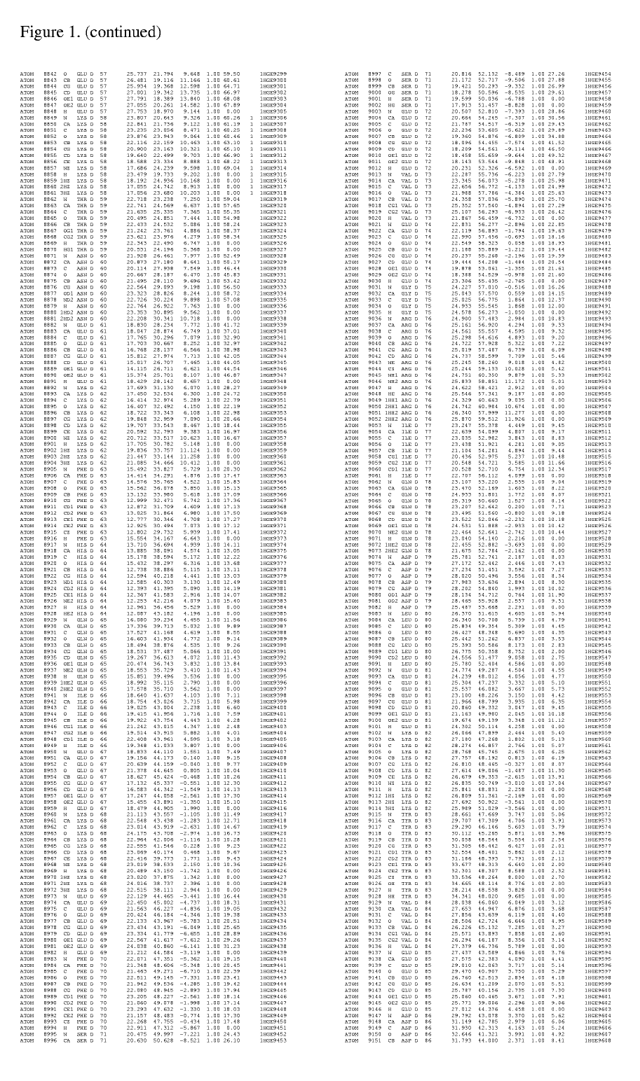

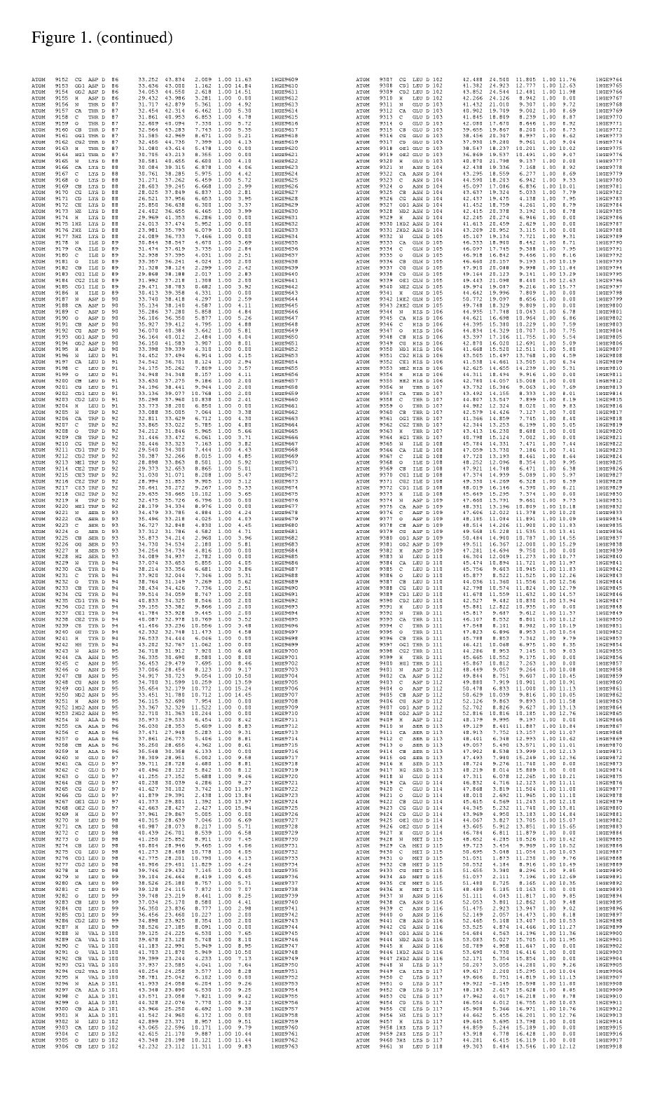

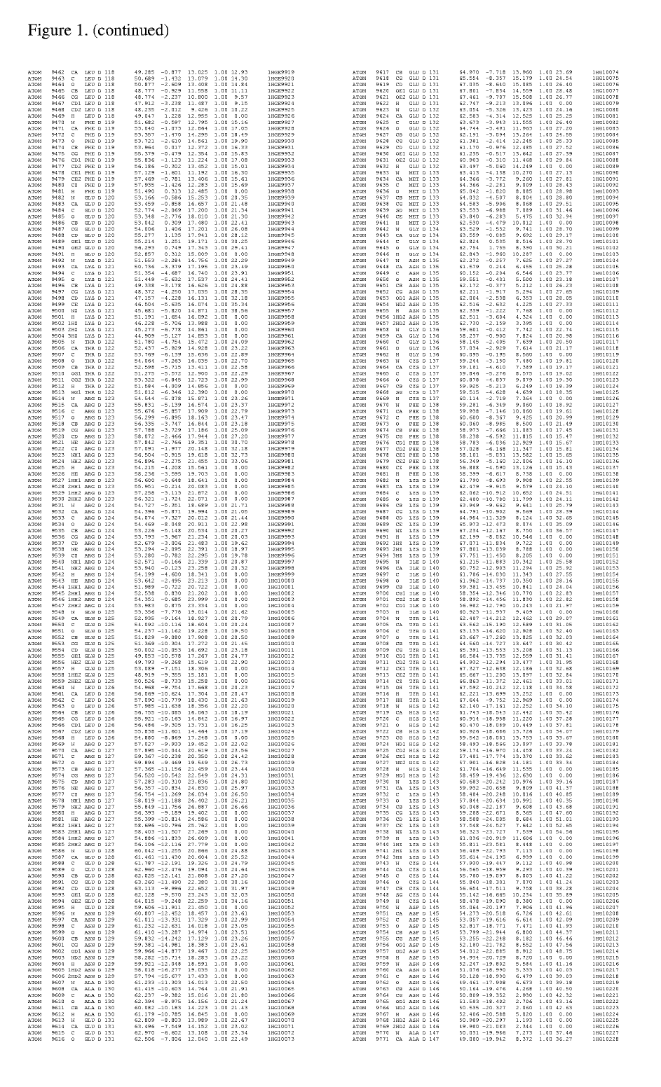

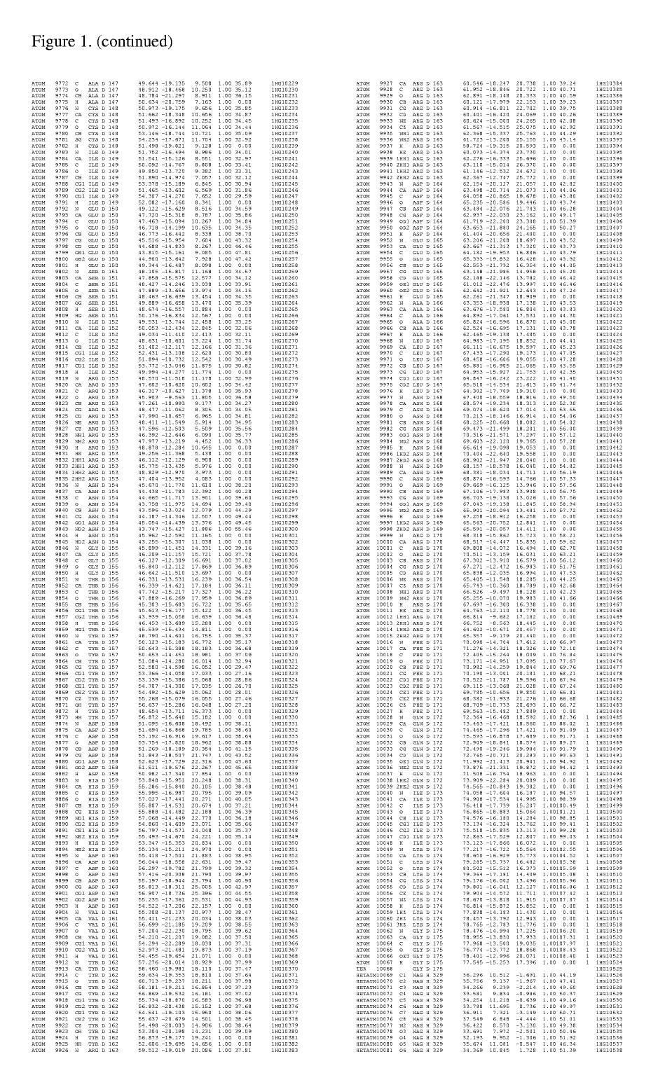

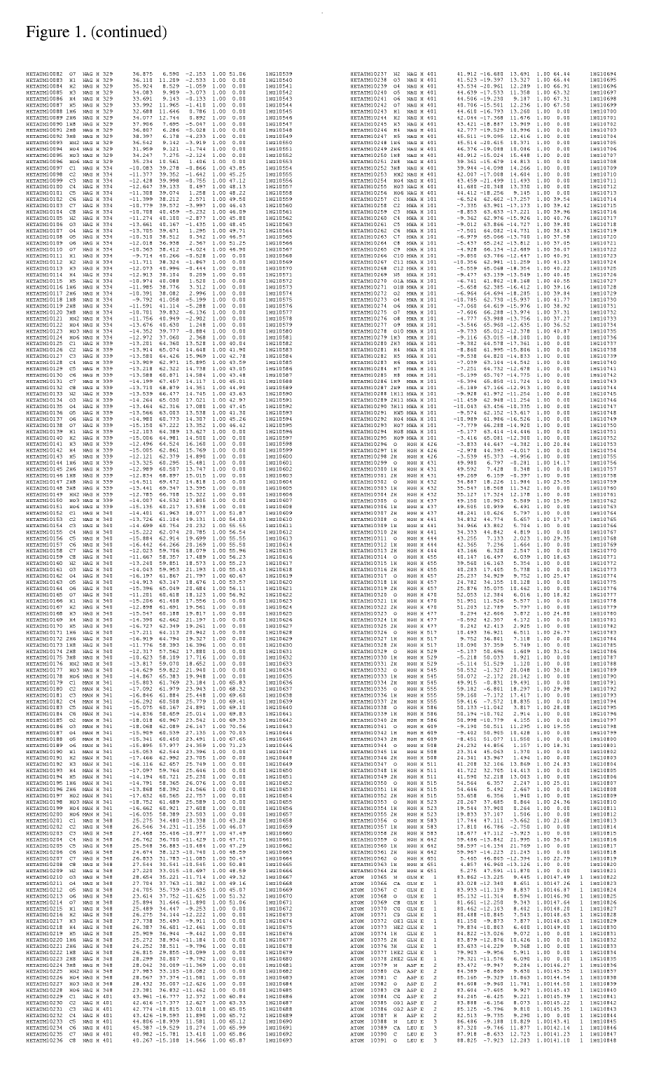

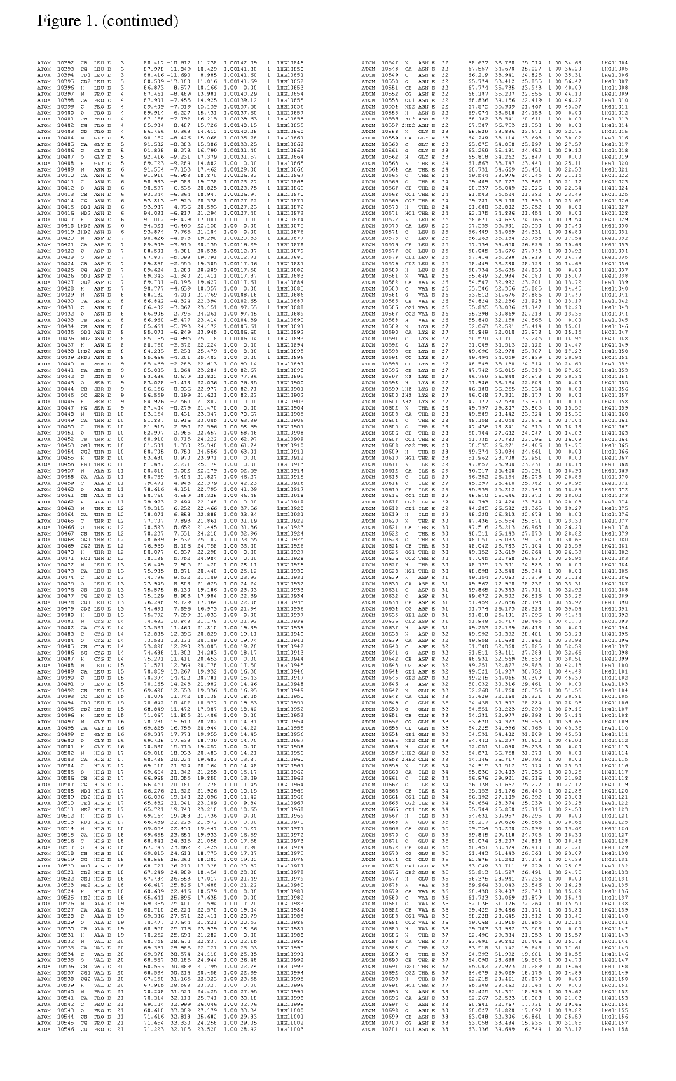

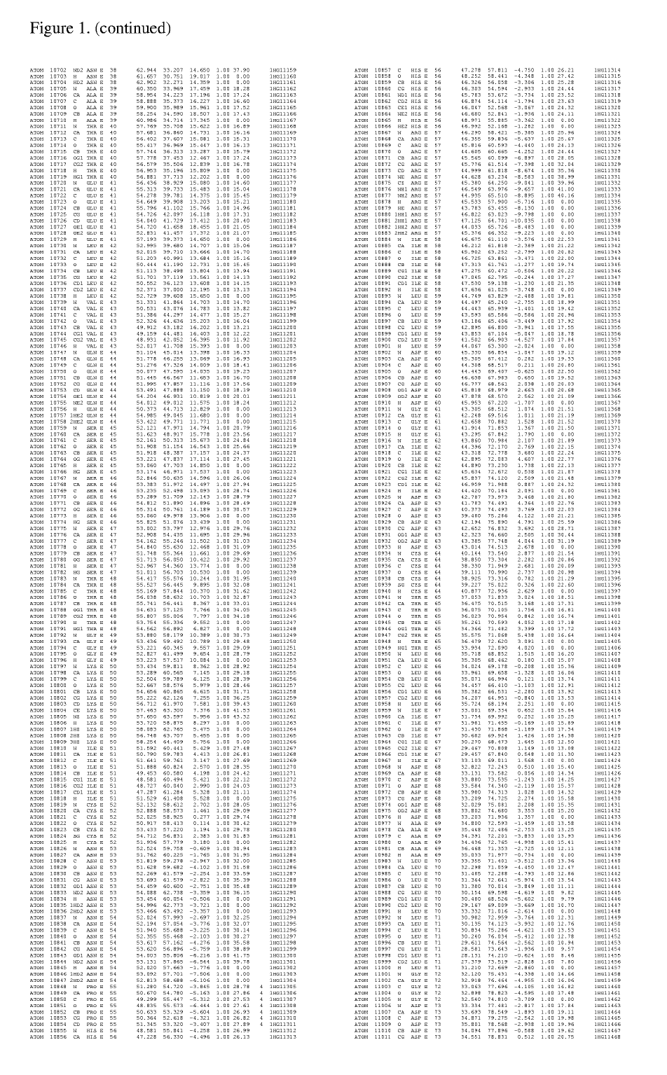

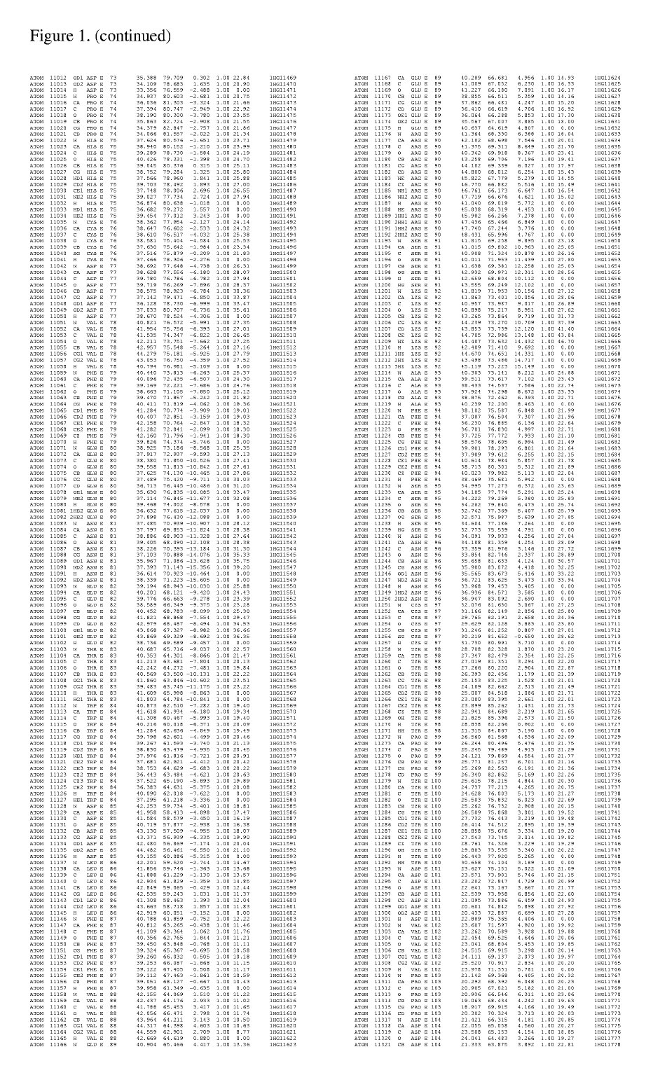

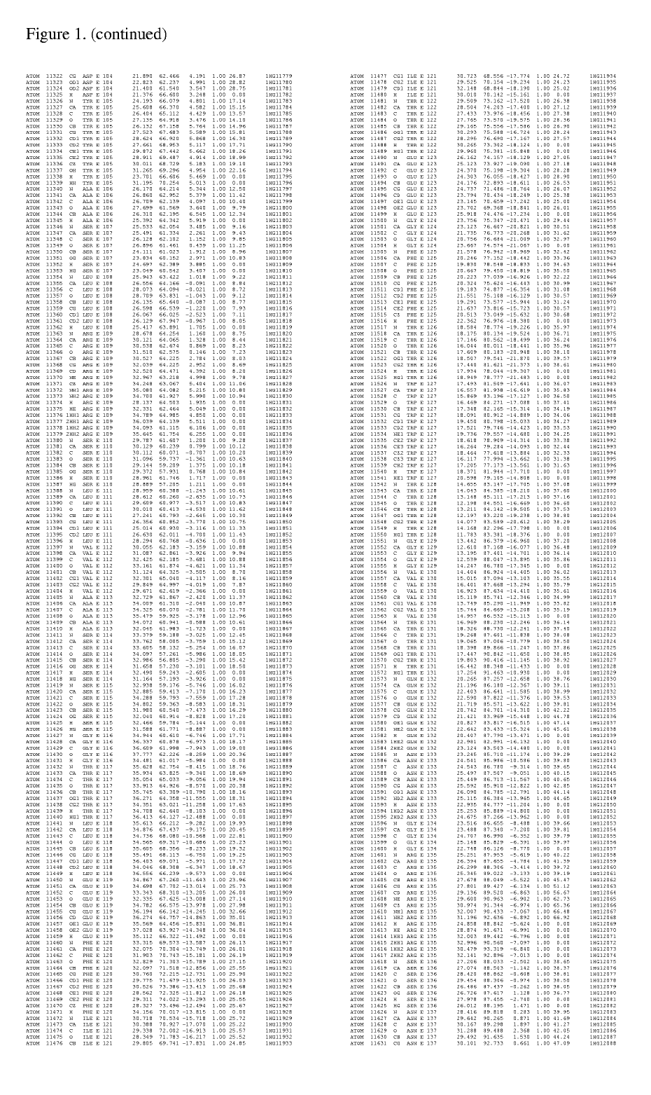

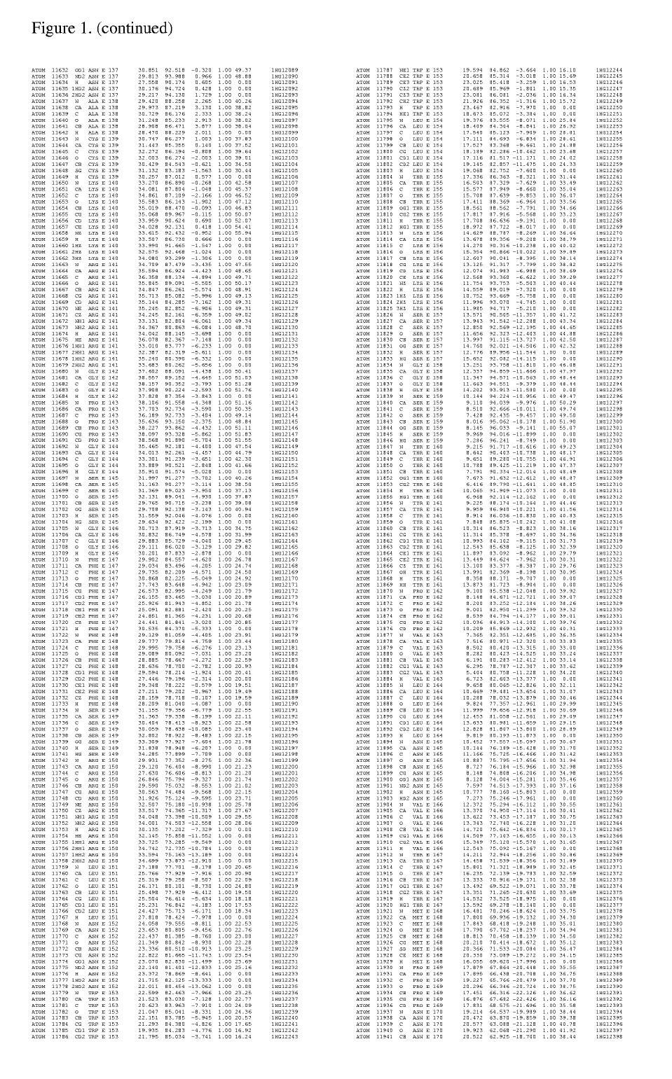

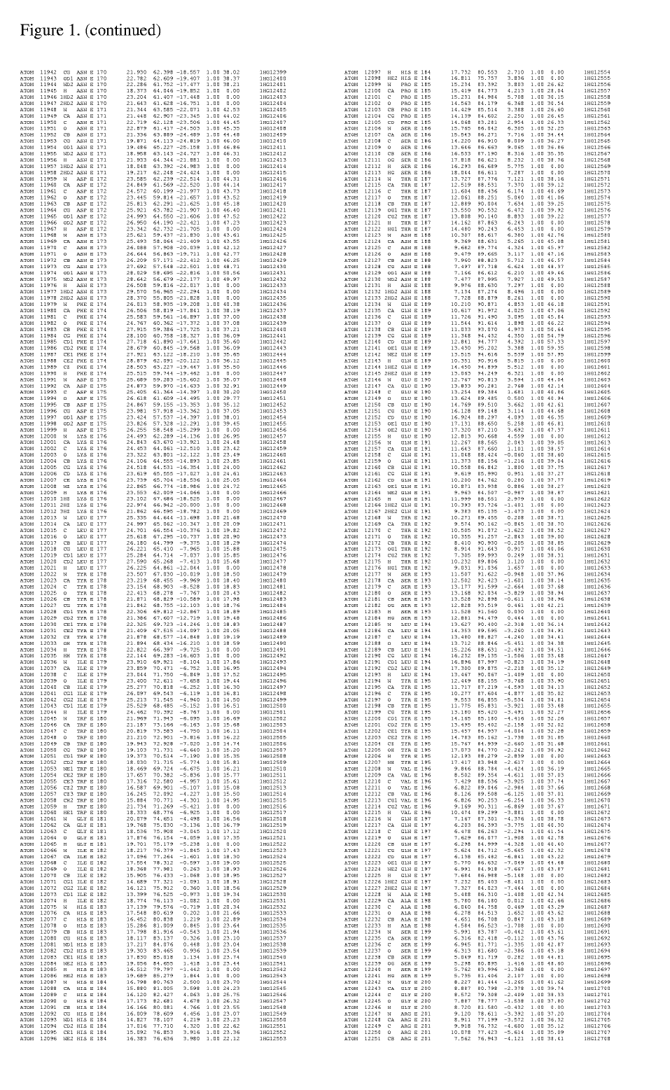

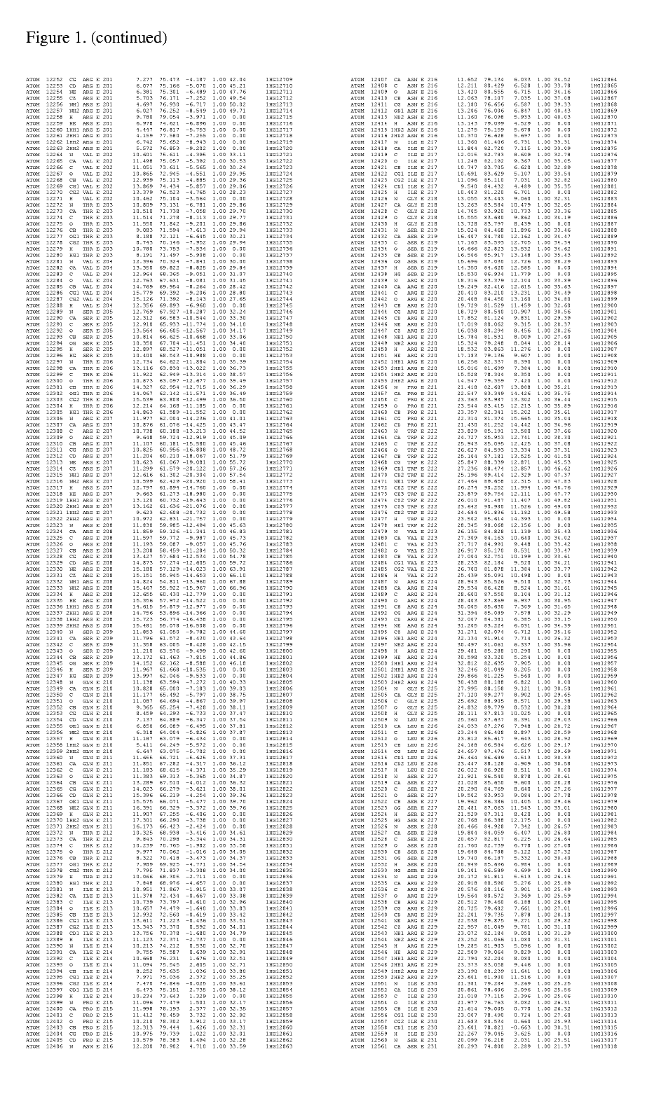

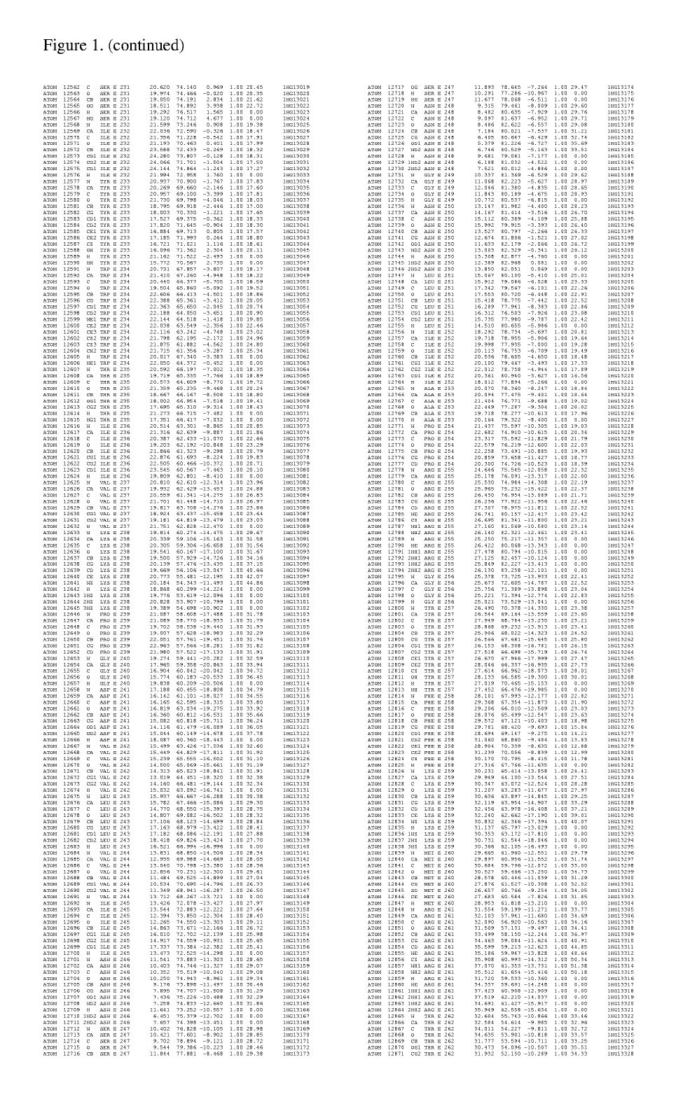

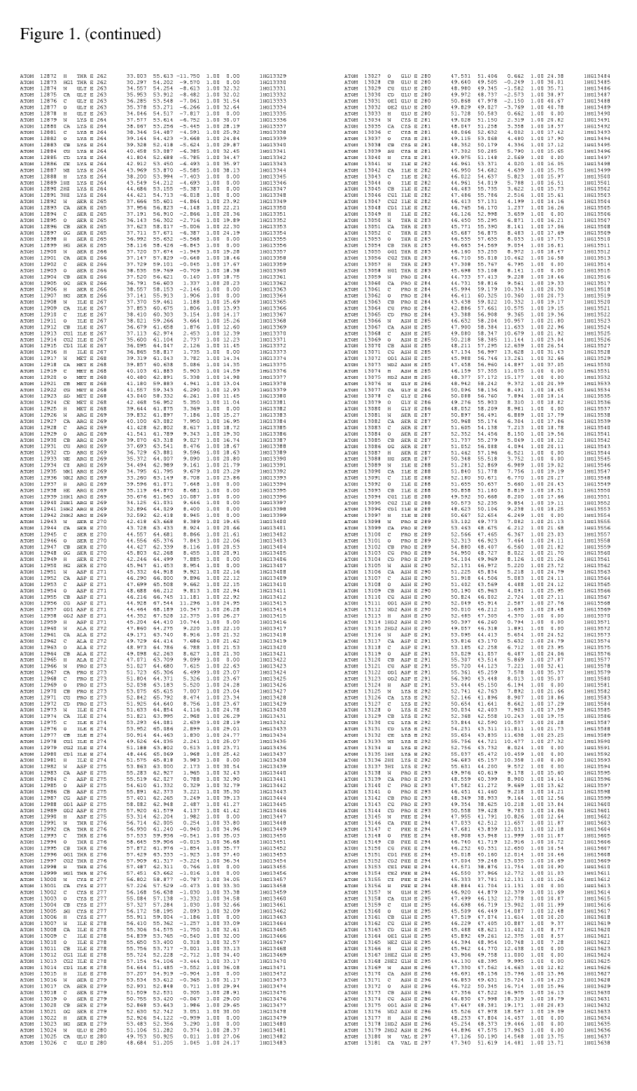

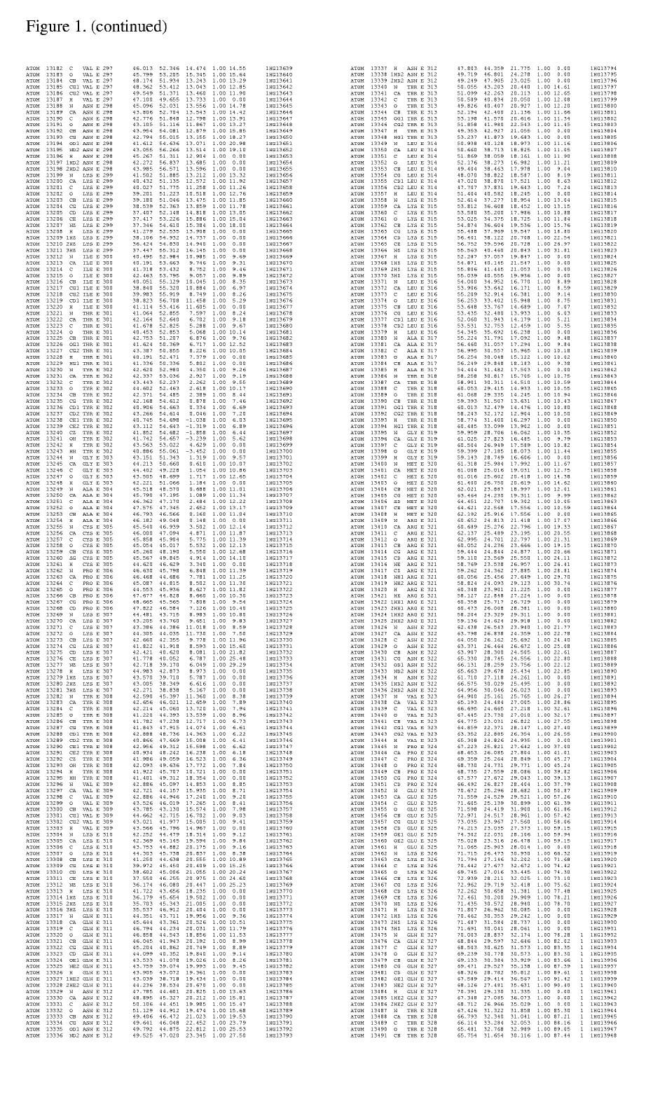

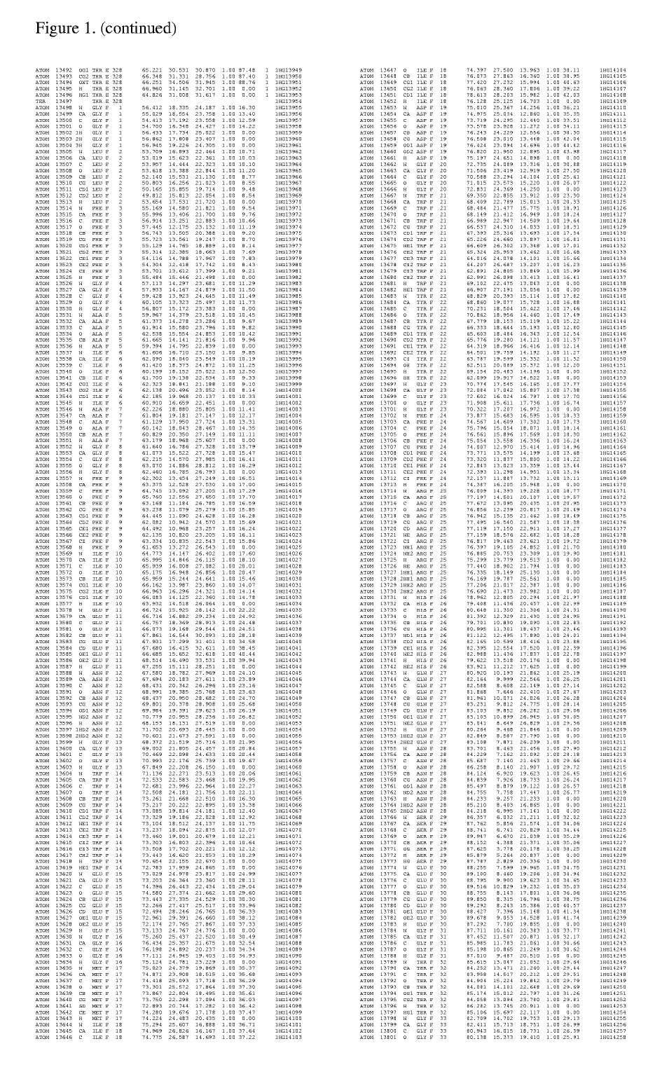

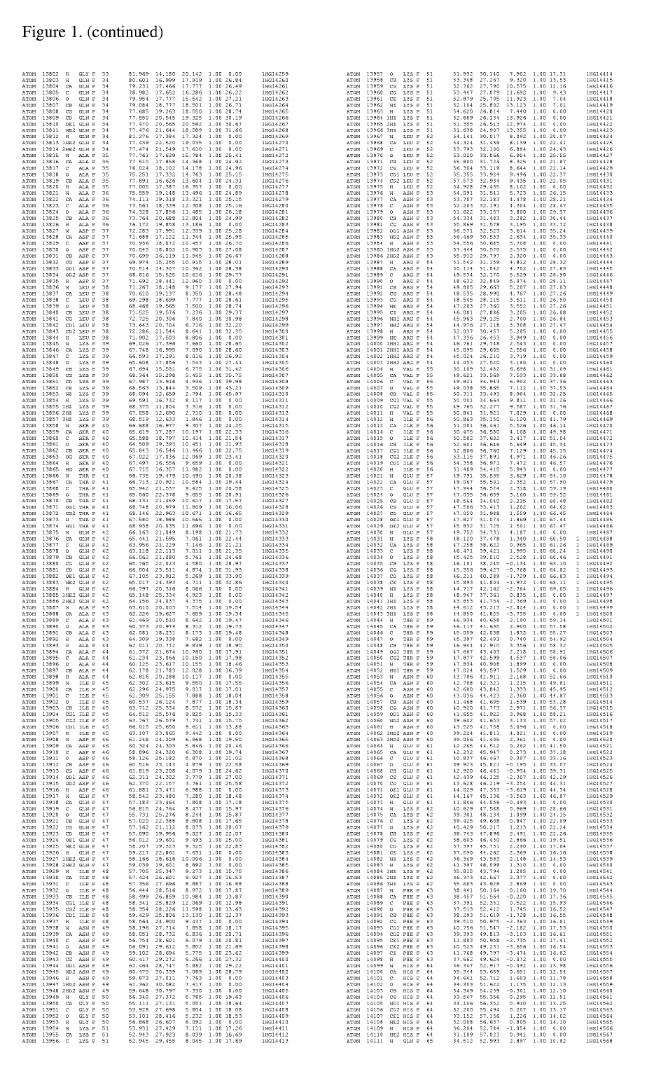

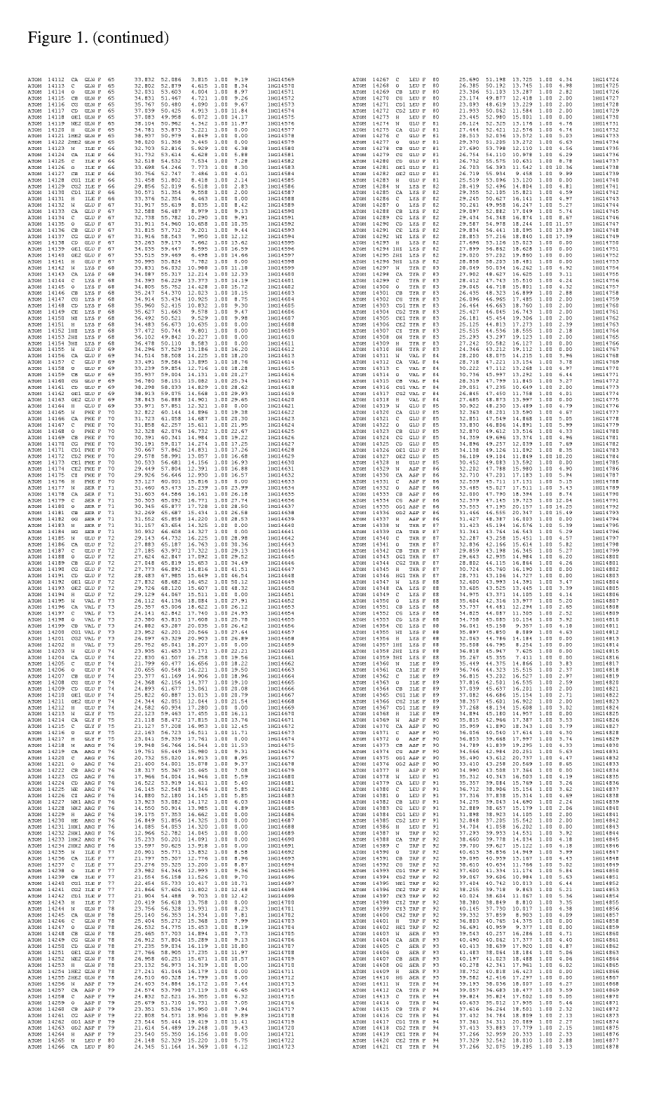

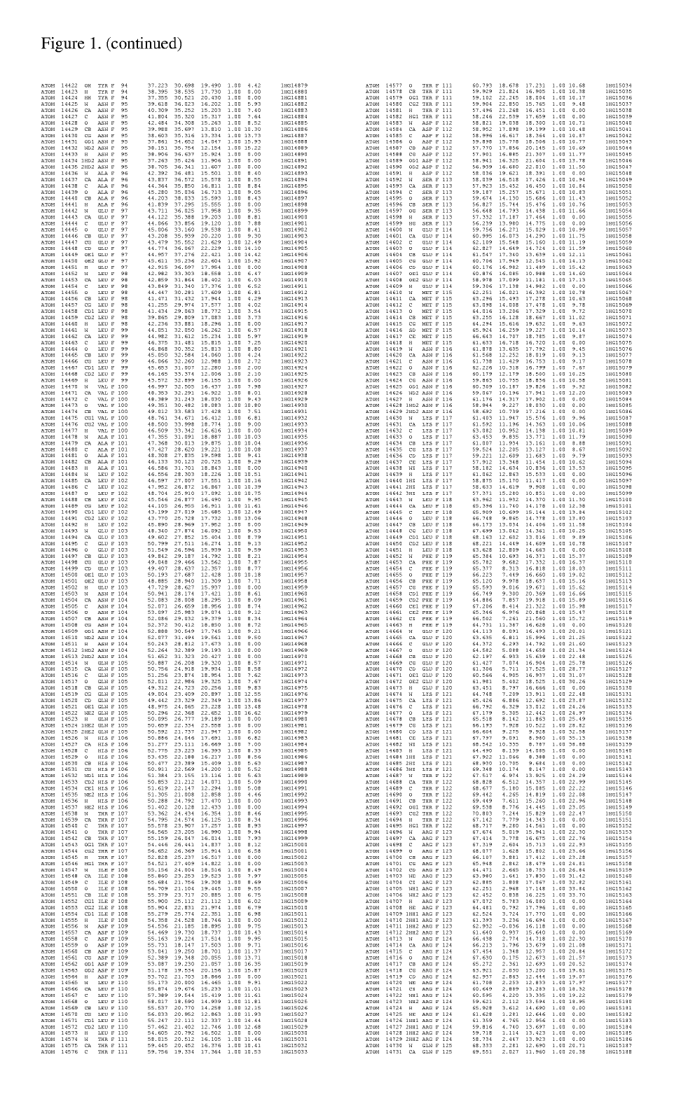

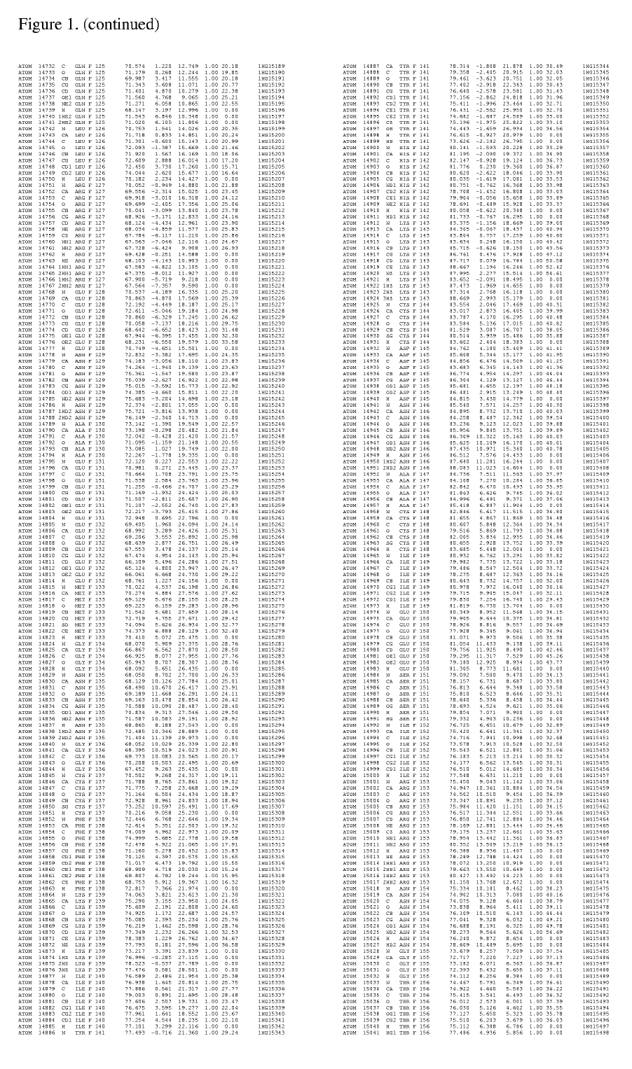

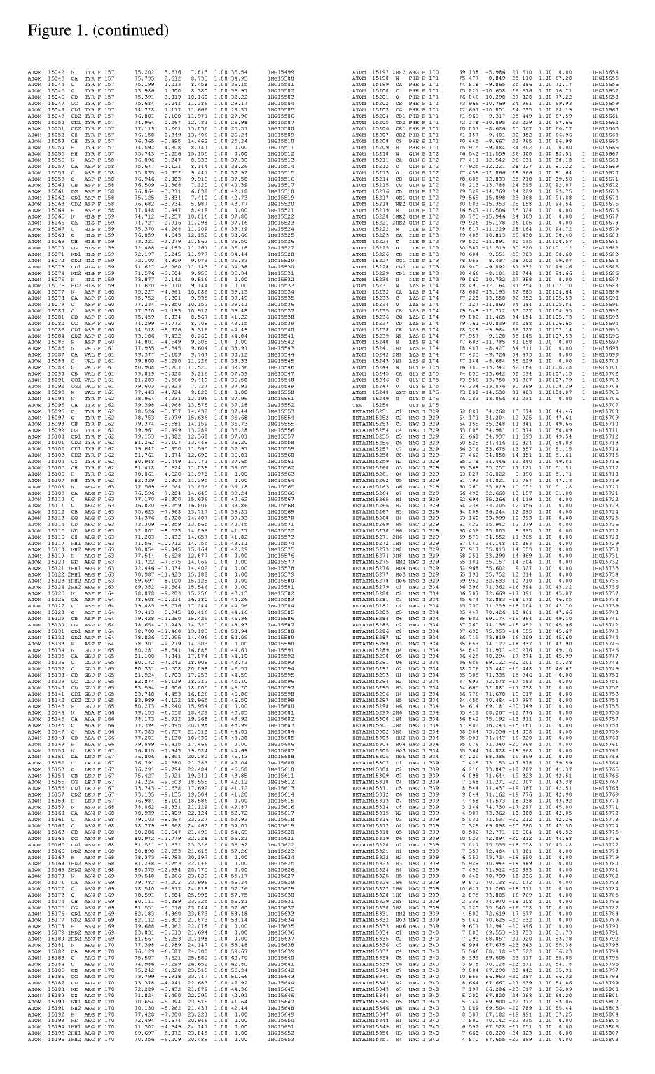

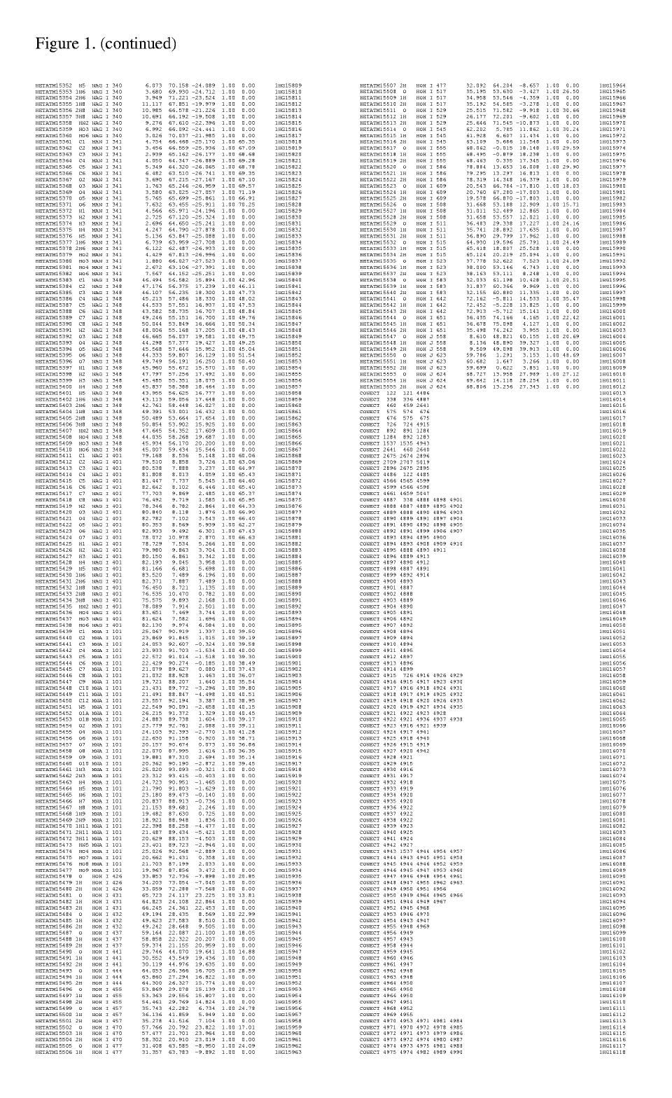

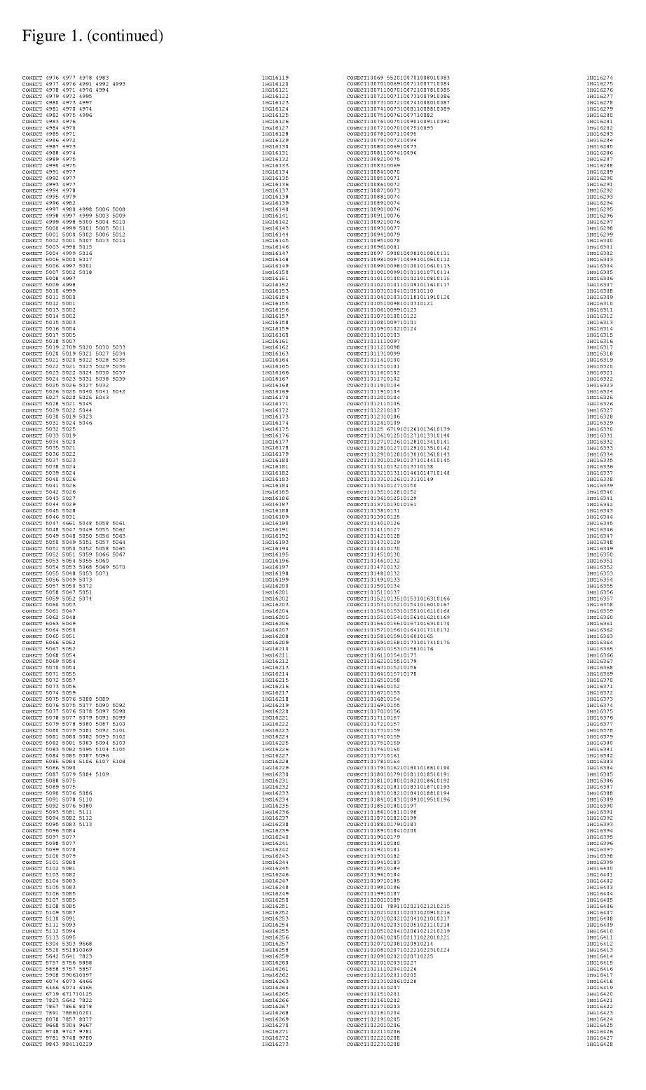

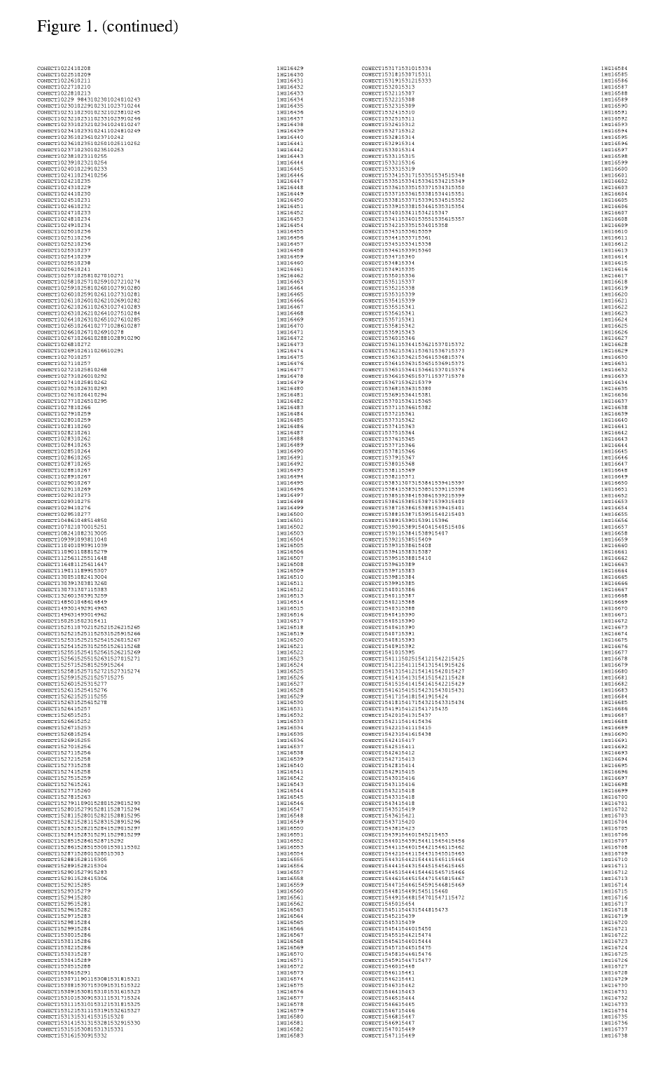

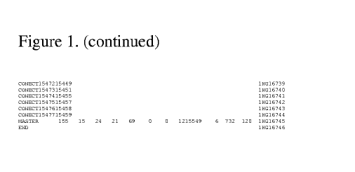

The three-dimensional structure of the large binding site of influenza hemagglutinin is defined by a set of structure coordinates as set forth in FIG. 1. The term “structure coordinates” refers to Cartesian coordinates derived from mathematical equations related to the patterns obtained on diffraction of a monochromatic beam of X-rays by the atoms (scattering centers) of the large binding site of influenza hemagglutinin in crystal form. The diffraction data are used to calculate an electron density map of the repeating unit of the crystal. The electron density maps are then used to establish the positions of the individual atoms of the large binding site of influenza hemagglutinin.

Those of skill in the art will understand that a set of structure coordinates for a protein or a protein-complex or a portion thereof, is a relative set of points that define a shape in three dimensions.

Thus, it is possible that an entirely different set of coordinates could define a similar or identical shape. Moreover, slight variations in the individual coordinates will have little effect on overall shape.

The variations in coordinates discussed above may be generated because of mathematical manipulations of the structure coordinates. For example, the structure coordinates set forth in FIG. 1 could be manipulated by crystallographic permutations of the structure coordinates, fractionalization of the structure coordinates, integer additions or subtractions to sets of the structure coordinates, inversion of the structure coordinates or any combination of the above.

Alternatively, modifications in the crystal structure due to mutations, additions, substitutions, and/or deletions of amino acids, or other changes in any of the components that make up the crystal could also account for variations in structure coordinates. If such variations are within an acceptable standard error as compared to the original coordinates, the resulting three-dimensional shape is considered to be the same.

Various computational analyses are therefore necessary to determine whether a molecule or molecular complex or a portion thereof is sufficiently similar to all or parts of the large binding site of influenza hemagglutinin described above as to be considered the same. Such analyses may be carried out in current software applications, such as the Molecular Similarity application of QUANTA (Molecular Simulations Inc., San Diego, Calif.) version 4.1, and as described in the accompanying User's Guide.

The Molecular Similarity application permits comparisons between different structures, different conformations of the same structure, and different parts of the same structure. The procedure used in Molecular Similarity to compare structures is divided into four steps: 1) load the structures to be compared; 2) define the atom equivalences in these structures; 3) perform a fitting operation; and 4) analyze the results.

Each structure is identified by a name. One structure is identified as the target (i.e., the fixed structure); all remaining structures are working structures (i.e., moving structures). Since atom equivalency within QUANTA is defined by user input, for the purpose of this invention we will define equivalent atoms as protein backbone atoms (N, C alpha, C and O) for all conserved residues between the two structures being compared. We will also consider only rigid fitting operations.

When a rigid fitting method is used, the working structure is translated and rotated to obtain an optimum fit with the target structure. The fitting operation uses an algorithm that computes the optimum translation and rotation to be applied to the moving structure, such that the root mean square difference of the fit over the specified pairs of equivalent atom is an absolute minimum. This number, given in angstroms, is reported by QUANTA.

For the purpose of this invention, any molecule or molecular complex that has a root mean square deviation of conserved residue backbone atoms (N, C alpha, C, O) of less than 1.5 angstrom when superimposed on the relevant backbone atoms described by structure coordinates listed in FIG. 1 are considered identical. More preferably, the root mean square deviation is less than 1.0 ångström.

The term “root mean square deviation” means the square root of the arithmetic mean of the squares of the deviations from the mean. It is a way to express the deviation or variation from a trend or object. For purposes of this invention, the “root mean square deviation” defines the variation in the backbone of a protein or protein complex from the relevant portion of the backbone of the large binding site of influenza hemagglutinin as defined by the structure coordinates described herein.

Once the structure coordinates of a protein crystal have been determined they are useful in solving the structures of other crystals.

Thus, in accordance with the present invention, the structure coordinates of the large binding site of influenza hemagglutinin, and portions thereof is stored in a machine-readable storage medium. Such data may be used for a variety of purposes, such as drug discovery and x-ray crystallographic analysis or protein crystal.

Accordingly, in one embodiment of this invention is provided a machine-readable data storage medium comprising a data storage material encoded with the structure coordinates set forth in FIG. 1.

For the first time, the present invention permits the use of structure-based or rational drug design techniques to design, select, and synthesize chemical entities, including inhibitory compounds that are capable of binding to the large binding site of influenza hemagglutinin, or any portion thereof.

One particularly useful drug design technique enabled by this invention is iterative drug design. Iterative drug design is a method for optimizing associations between a protein and a compound by determining and evaluating the three-dimensional structures of successive sets of protein/compound complexes.

Those of skill in the art will realize that association of natural ligands or substrates with the binding pockets of their corresponding receptors or enzymes is the basis of many biological mechanisms of action. The term “binding site”, as used herein, refers to a region of a molecule or molecular complex, that, as a result of its shape, favorably associates with another chemical entity or compound. Similarly, many drugs exert their biological effects through association with the binding pockets of receptors and enzymes. Such associations may occur with all or any parts of the binding pockets. An understanding of such associations will help lead to the design of drugs having more favorable associations with their target receptor or enzyme, and thus, improved biological effects. Therefore, this information is valuable in designing potential ligands or inhibitors of receptors or enzymes, such as blockers of hemagglutinin.

The term “associating with” or “interacting with” refers to a condition of proximity between chemical entities or compounds, or portions thereof. The association or interaction may be non-covalent, wherein the juxtaposition is energetically favored by hydrogen bonding or van der Waals or electrostatic interactions, or it may be covalent.

In iterative drug design, crystals of a series of protein/compound complexes are obtained and then the three-dimensional structures of each complex is solved. Such an approach provides insight into the association between the proteins and compounds of each complex. This is accomplished by selecting compounds with inhibitory activity, obtaining crystals of this new protein/compound complex, solving the three-dimensional structure of the complex, and comparing the associations between the new protein/compound complex and previously solved protein/compound complexes. By observing how changes in the compound affected the protein/compound associations, these associations may be optimized.

In some cases, iterative drug design is carried out by forming successive protein-compound complexes and then crystallizing each new complex. Alternatively, a pre-formed protein crystal is soaked in the presence of an inhibitor, thereby forming a protein/compound complex and obviating the need to crystallize each individual protein/compound complex. Advantageously, the large binding site of influenza hemagglutinin crystals, may be soaked in the presence of a compound or compounds, such as hemagglutinin inhibitors, to provide hemagglutinin/ligand crystal complexes.

As used herein, the term “soaked” refers to a process in which the crystal is transferred to a solution containing the compound of interest.

### The Storage Medium

The storage medium in which the atomic co-ordinates are provided is preferably random access memory (RAM), but may also be read-only memory (ROM e.g. CDROM), or a diskette. The storage medium may be local to the computer, or may be remote (e.g. a networked storage medium, including the internet).

The invention also provides a computer-readable medium for a computer, characterised in that the medium contains atomic co-ordinates of the large binding site of influenza hemagglutinin.

The atomic co-ordinates are preferably those set forth in FIG. 1, or variants thereof.

Any suitable computer can be used in the present invention.

### Molecular Modelling Techniques

Molecular modelling techniques can be applied to the atomic co-ordinates of the large binding site of influenza hemagglutinin to derive a range of 3D models and to investigate the structure of ligand binding sites. A variety of molecular modelling methods are available to the skilled person for use according to the invention [e.g. ref. 5].

At the simplest level, visual inspection of a computer model of the large binding site of influenza hemagglutinin can be used, in association with manual docking of models of functional groups into its binding sites.

Software for implementing molecular modelling techniques may also be used. Typical suites of software include CERIUS2 [Available from Molecular Simulations Inc], SYBYL [Available from Tripos Inc], AMBER [Available from Oxford Molecular], HYPERCHEM [Available from Hypercube Inc], INSIGHT II [Available from Molecular Simulations Inc], CATALYST [Available from Molecular Simulations Inc], CHEMSITE [Available from Pyramid Learning], QUANTA [Available from Molecular Simulations Inc]. These packages implement many different algorithms that may be used according to the invention (e.g. CHARMm molecular mechanics [Brooks et al. (1983) J. Comp. Chem. 4: 187-217]). Their uses in the methods of the invention include, but are not limited to: (a) interactive modelling of the structure with concurrent geometry optimisation (e.g. QUANTA); (b) molecular dynamics simulation of the large binding site of influenza hemagglutinin (e.g. CHARMM, AMBER); (c) normal mode dynamics simulation of the large binding site of influenza hemagglutinin (e.g. CHARMM).

Modelling may include one or more steps of energy minimisation with standard molecular mechanics force fields, such as those used in CHARMM and AMBER.

These molecular modelling techniques allow the construction of structural models that can be used for in silico drug design and modelling.

### De Novo Compound Design

The molecular modelling steps used in the methods of the invention may use the atomic co-ordinates of the large binding site of influenza hemagglutinin, and models derived therefrom, to determine binding surfaces.

This preferably reveals van der Waals contacts, electrostatic interactions, and/or hydrogen bonding opportunities.

These binding surfaces will typically be used by grid-based techniques (e.g. GRID [Goodford (1985) J. Med. Chem. 28: 849-857], CERIUS2) and/or multiple copy simultaneous search (MCSS) techniques to map favourable interaction positions for functional groups. This preferably reveals positions in the large binding site of influenza hemagglutinin for interactions such as, but not limited to, those with protons, hydroxyl groups, amine groups, hydrophobic groups (e.g. methyl, ethyl, benzyl) and/or divalent cations.

Once functional groups or small molecule fragments which can interact with specific sites in the binding surface of the large binding site of influenza hemagglutinin have been identified, they can be linked in a single compound using either bridging fragments with the correct size and geometry or frameworks which can support the functional groups at favourable orientations, thereby providing a compound according to the invention. Whilst linking of functional groups in this way can be done manually, perhaps with the help of software such as QUANTA or SYBYL, the following software may be used for assistance: HOOK [Available from Molecular Simulations Inc], which links multiple functional groups with molecular templates taken from a database, and/or CAVEAT [Lauri & Bartlett (1994) Comp. Aided Mol. Design. 8: 51-66], which designs linking units to constrain acyclic molecules.

Other computer-based approaches to de novo compound design that can be used with the large binding site of influenza hemagglutinin atomic co-ordinates include LUDI [Available from Molecular Simulations Inc], SPROUT [Available from http://chem.leeds.ac.uk/ICAMS/SPROUT.html] and LEAPFROG [Available from Tripos Inc].

### Pharmacophore Searching

As well as using de novo design, a pharmacophore of the large binding site of influenza hemagglutinin can be defined i.e. a collection of chemical features and 3D constraints that expresses specific characteristics responsible for biological activity. The pharmacophore preferably includes surface-accessible features, more preferably including hydrogen bond donors and acceptors, charged/ionisable groups, and/or hydrophobic patches. These may be weighted depending on their relative importance in conferring activity.

Pharmacophores can be determined using software such as CATALYST (including HypoGen or HipHop) [Available from Molecular Simulations Inc], CERIUS2, or constructed by hand from a known conformation of a lead compound. The pharmacophore can be used to screen in silico compound libraries, using a program such as CATALYST [Available from Molecular Simulations Inc].

Suitable in silico libraries include the Available Chemical Directory (MDL Inc), the Derwent World Drug Index (WDI), BioByteMasterFile, the National Cancer Institute database (NCI), and the Maybridge catalog.

### Docking

Compounds in these in silico libraries can also be screened for their ability to interact with the large binding site of influenza hemagglutinin by using their respective atomic co-ordinates in automated docking algorithms.

Suitable docking algorithms include: DOCK [Kuntz et al. (1982) J. Mol. Biol. 161: 269-288], AUTODOCK [Available from Oxford Molecular], MOE-DOCK [Available from Chemical Computing Group Inc.] or FLEXX [Available from Tripos Inc.].

Docking algorithms can also be used to verify interactions with ligands designed de novo.

In this invention the terms “analog” and “derivative” are defined as follows. According to the present invention it is possible to design structural analogs or derivatives of the influenza virus binding oligosaccharide sequences. Thus, the invention is also directed to the structural analogs of the substances according to the invention. The structural analogs according to the invention comprises the structural elements important for the binding of influenza virus to the oligosaccharide sequences. For design of effective structural analogs it is necessary to know the structural element important for the binding between influenza virus and the saccharides. The important structural elements are preferably not modified or these are modified by a very close mimetic of the important structural element.

The structural derivatives according to the invention are oligosaccharide sequences according to the invention modified chemically so that the binding to the influenza virus is retained or increased. According to the invention it is preferred to derivatize one or several of the hydroxyl or acetamido groups of the oligosaccharide sequences. The invention describes several positions of the molecules which could be changed when preparing the analogs or the derivatives. The hydroxyl or acetamido groups which preferably tolerate at least certain modifications are self-evident for a skilled artisan from the formulas described herein.

Bulky or acidic substituents and other structures, such as monosaccharide residues, are not tolerated, but methods to produce oligosaccharide analogs e.g. for the binding of a lectin are well known. For example, numerous analogs of sialyl-Lewis×oligosaccharide has been produced, representing the active functional groups different scaffold, see page 12090 Sears and Wong 1996. Similarly analogs of heparin oligosaccharides has been produced by Sanofi corporation and sialic acid mimicking inhibitors such as Zanamivir and Tamiflu (Relenza) for the sialidase enzyme by numerous groups. Preferably the oligosaccharide analog is build on a molecule comprising at least one six- or five-membered ring structure, more preferably the analog contains at least two ring structures comprising 6 or 5 atoms. In mimicking structures monosaccharide rings may be replaced rings such as cyclohexane or cyclopentane, aromatic rings including benzene ring, heterocyclic ring structures may comprise beside oxygen for example nitrogen and sulphur atoms. To lock the active ring conformations the ring structures may be interconnected by tolerated linker groups. Typical mimetic. structure may also comprise peptide analog-structures for the oligosaccharide sequence or part of it.

The effects of the active groups to binding activity are cumulative and lack of one group could be compensated by adding an active residue on the other side of the molecule. Molecular modelling, preferably by a computer can be used to produce analog structures for the influenza virus binding oligosaccharide sequences according to the invention. The results from the molecular modelling of several oligosaccharide sequences are given in examples and the same or similar methods, besides NMR and X-ray crystallography methods, can be used to obtain structures for other oligosaccharide sequences according to the invention. To find analogs the oligosaccharide structures can be “docked” to the carbohydrate binding molecule(s) of influenza virus, most probably to lectins of the virus and possible additional binding interactions can be searched.

It is also noted that the monovalent, oligovalent or polyvalent oligosaccharides can be activated to have higher activity towards the lectins by making derivative of the oligosaccharide by combinatorial chemistry. When the library is created by substituting one or few residues in the oligosaccharide sequence, it can be considered as derivative library, alternatively when the library is created from the analogs of the oligosaccharide sequences described by the invention. A combinatorial chemistry library can be built on the oligosaccharide or its precursor or on glycoconjugates according to the invention. For example, oligosaccharides with variable reducing end can be produced by so called carbohydrid technology.

In a preferred embodiment a combinatorial chemistry library is conjugated to the influenza virus binding substances described by the invention. In a more preferred embodiment the library comprises at least 6 different molecules. Such library is preferred for use of assaying microbial binding to the oligosaccharide sequences according to the invention. A high affinity binder could be identified from the combinatorial library for example by using an inhibition assay, in which the library compounds are used to inhibit the viral binding to the glycolipids or glycoconjugates described by the invention. Structural analogs and derivatives preferred according to the invention can inhibit the binding of the influenza virus binding oligosaccharide sequences according to the invention to influenza virus.

In the following the influenza virus binding sequence is described as an oligosaccharide sequence. The oligosaccharide sequence defined here can be a part of a natural or synthetic glycoconjugate or a free oligosaccharide or a part of a free oligosaccharide. Such oligosaccharide sequences can be bonded to various monosaccharides or oligosaccharides or polysaccharides on polysaccharide chains, for example, if the saccharide sequence is expressed as part of a viral polysaccharide. Moreover, numerous natural modifications of monosaccharides are known as exemplified by O-acetyl or sulphated derivative of oligosaccharide sequences. The influenza virus binding substance defined here can comprise the oligosaccharide sequence described as a part of a natural or synthetic glycoconjugate or a corresponding free oligosaccharide or a part of a free oligosaccharide. The influenza virus binding substance can also comprise a mix of the influenza virus binding oligosaccharide sequences.

The influenza virus binding oligosaccharide sequences can be synthesized enzymatically by glycosyltransferases, or by transglycosylation catalyzed by glycosidase or transglycosidase enzymes (Ernst et al., 2000). Specifities of these enzymes and the use of co-factors can be engineered. Specific modified enzymes can be used to obtain more effective synthesis, for example, glycosynthase is modified to do transglycosylation only. Organic synthesis of the saccharides and the conjugates described herein or compounds similar to these are known (Ernst et al., 2000). Saccharide materials can be isolated from natural sources and modified chemically or enzymatically into the influenza virus binding compounds. Natural oligosaccharides can be isolated from milks produced by various ruminants. Transgenic organisms, such as cows or microbes, expressing glycosylating enzymes can be used for the production of saccharides.

The virus binding substances are preferably represented in clustered form such as by glycolipids on cell membranes, micelles, liposomes, or on solid phases such as TCL-plates used in the assays. The clustered representation with correct spacing creates high affinity binding.

According to the invention it is also possible to use the influenza virus binding epitopes or naturally occurring, or a synthetically produced analogue or derivative thereof having a similar or better binding activity with regard to influenza virus. It is also possible to use a substance containing the virus binding substance such as a receptor active ganglioside described in the invention or an analogue or derivative thereof having a similar or better binding activity with regard to influenza virus. The virus binding substance may be a glycosidically linked terminal epitope of an oligosaccharide chain. Alternatively the virus binding epitope may be a branch of an oligosaccharide chain, preferably a polylactosamine chain.

The influenza virus binding substance may be conjugated to an antibiotic substance, preferably a penicillin type antibiotic. The influenza virus binding substance targets the antibiotic to bacterium causing secondary infections due to influenza virus. Such conjugate is beneficial in treatment because a lower amount of antibiotic is needed for treatment or therapy against secondary infectants, which leads to lower side effect of the antibiotic. The antibiotic part of the conjugate is aimed at killing or weaken the bacteria, but the conjugate may also have an antiadhesive effect as described below.

The virus binding substances, preferably in oligovalent or clustered form, can be used to treat a disease or condition caused by the presence of the influenza virus. This is done by using the influenza virus binding substances for anti-adhesion, i.e. to inhibit the binding of influenza virus to the receptor epitopes of the target cells or tissues. When the influenza virus binding substance or pharmaceutical composition is administered it will compete with receptor glycoconjugates on the target cells for the binding of the virus. Some or all of the virus will then be bound to the influenza virus binding substance instead of the receptor on the target cells or tissues. The virus bound to the influenza virus binding substances are then removed from the patient (for example by the fluid flow in the gastrointestinal tract), resulting in reduced effects of the virus on the health of the patient. Preferably the substance used is a soluble composition comprising the influenza virus binding substances. The substance can be attached to a carrier substance which is preferably not a protein. When using a carrier molecule several molecules of the influenza virus binding substance can be attached to one carrier and inhibitory efficiency is improved.

The term “treatment” used herein relates both to treatment in order to cure or alleviate a disease or a condition, and to treatment in order to prevent the development of a disease or a condition. The treatment may be either performed in an acute or in a chronic way.

The pharmaceutical composition according to the invention may also comprise other substances, such as an inert vehicle, or pharmaceutically acceptable carriers, preservatives etc., which are well known to persons skilled in the art.

The substance or pharmaceutical composition according to the invention may be administered in any suitable way, although an oral or nasal administration especially in the form of a spray or inhalation are preferred. The nasal and oral inhalation and spray dosage technologies are well-known in the art. The preferred dose depend on the substance and the infecting virus. In general dosages between 0.01 mg and 500 mg are preferred, more preferably the dose is between 0.1 mg and 50 mg. The dose is preferably administered at least once daily, more preferably twice per day and most preferably three or four times a day. In case of excessive secretion of mucus and sneezing or cough the dosage may be increased with 1-3 doses a day.

The present invention is directed to novel divalent molecules as substances. Preferred substances includes preferred molecules comprising the flexible spacer structures and peptide and/or oxime linkages. The present invention is further directed to the novel uses of the molecules as medicines. The present invention is further directed to in methods of treatments applying the substances according to the invention.

The term “patient”, as used herein, relates to any human or non-human mammal in need of treatment according to the invention.

Glycolipid and carbohydrate nomenclature is according to recommendations by the IUPAC-IUB Commission on Biochemical Nomenclature (Carbohydrate Res. 1998, 312, 167; Carbohydrate Res. 1997, 297, 1; Eur. J. Biochem. 1998, 257, 29).

It is assumed that Gal, Glc, GlcNAc, and Neu5Ac are of the D-configuration, Fuc of the L-configuration, and all the monosaccharide units in the pyranose form. Glucosamine is referred as GlcN or GlcNH₂ and galactosamine as GalN or GalNH₂. Glycosidic linkages are shown partly in shorter and partly in longer nomenclature, the linkages of the Neu5Ac-residues β3 and α6 mean the same as α2-3 and α2-6, respectively, and with other monosaccharide residues α1-3, β1-3, β1-4, and β1-6 can be shortened as α3, β3, β4, and β6, respectively. Lactosamine refers to N-acetyllactosamine, Galβ4GlcNAc, and sialic acid is N-acetylneuraminic acid (Neu5Ac, NeuNAc or NeuAc) or N-glycolylneuraminic acid (Neu5Gc) or any other natural sialic acid. Term glycan means here broadly oligosaccharide or polysaccharide chains present in human or animal glycoconjugates, especially on glycolipids or glycoproteins. In the shorthand nomenclature for fatty acids and bases, the number before the colon refers to the carbon chain length and the number after the colon gives the total number of double bonds in the hydrocarbon chain.

### EXAMPLES

### Example 1

### Modeling Studies of the Influenza Hemagglutinin

Introduction—The X-ray crystallographic structure of the hemagglutinin of the X-31 strain of human influenza virus was used for the docking (PDB-database, www.rcsb.org/pdp, the database structure 1HGE, FIG. 1.). The structure used in the modelling is a complex structure including Neu5Acα-OMe at the primary sialic acid binding site, the large oligosaccharide modelled to the site had one Neu5Acα-superimposable to the one in the 1HGE, but glycosidic glycan instead of the methyl group. The studies and sequence analyses described below in conjunction with hemagglutination-inhibition studies used for evaluation of the binding efficacy of the different branched poly-N-acetylactosamine inhibitors. The basic hemagglutinin structure consists of a trimer comprising the two subunits HA1 and HA2, the first of which contains the primary sialic acid binding site.

In addition to the primary site, which binds to both sialyl-α3-lactose and sialyl-α6-lactose, a secondary site exists which has been previously found to bind sialyl-α3-lactose as well but not sialyl-α6-lactose.

Results—Docking of the best binding inhibitory structures was performed under the premise that the primary sialic acid site of the hemagglutinin serves as the nucleation point from which the rest of the oligosaccharide folds itself onto the protein surface. From previous crystal structures of various complexes with small linear oligosaccharides and a branched structure it was obvious that maximally three sugars could be accommodated within the primary site and that further sugars will force the oligosaccharide to fold itself in different directions outside the primary site depending on the actual structure. The only structurally relevant branched compound investigated so far is

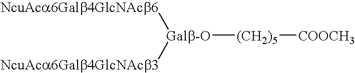

for which only the three terminal sugars of one of the branches is visible in the crystal structure and where the GlcNAc residue is seen to double back placing it on top of the NeuAc residue.

Of the various branched type 2-based disialylated oligosaccharides produced by Carbion for testing of their inhibitory power in the hemagglutination assay, two structures stood out for clearly stronger binding effectivity than the other isomers of similar size:

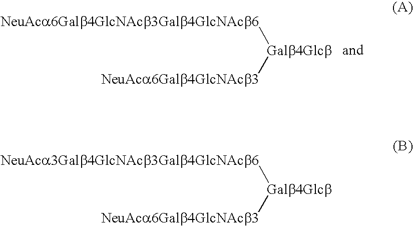

For these larger branched disialylated oligosaccharide structures the topography of the protein surface, the distribution of mutations of residues noncritical for binding from a large number of strains (see below) as well as the existence of a secondary site located within reach of the structures in question, suggested an oligosaccharide fold that would have to involve both the primary and secondary sites and that as a further prerequisite the NeuAc residue in the primary site would have to be α6-linked.

With these considerations in mind it was found that the two structures given above could be manually docked into both the primary and secondary sites without building any strain into either the oligosaccharides or the protein structure, meaning that only energetically favorable conformations around the constituent disaccharide glycosidic linkages as documented earlier in the literature had to be employed. Ensuing energy minimizations and dynamics simulations of these two complexes yielded the pictures shown below.

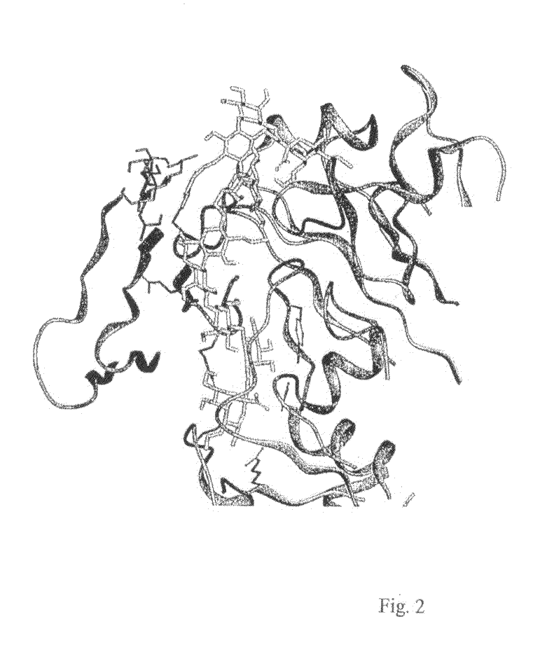

In the FIG. 2 the oligosaccharide having both NeuAc residues α6-linked is shown with the sialic acid of the shorter branch in the primary site at the top of the protein and the other sialic acid at the bottom in the pocket of the secondary site. Although the sialic acid interacts with some amino acid side chains that are identical to those found in the NeuAcα3Galβ4Glc complex an exact superposition cannot be attained since the oligosaccharide is in its most extended conformation leaving the NeuAcα6 residue 2-3 Å above the corresponding NeuAcα3 residue of the trisaccharide. Regarding the oligosaccharide having a NeuAcα3 residue attached at the longer branch a very similar picture is arrived at except of course for the sialic acid itself (not shown). It is noteworthy that the NeuAcα3 residue could be accommodated in the binding pocket without any repositioning of the oligosaccharide chain or perturbation of the protein structure, suggesting that the docked structures may be close to the actual complexes.

Further evidence for the probability of the docked structures being relevant for the true complexes comes from comparative hemagglutination-inhibition studies using structure (B) and different strains of the virus.

| | | Hemagglutination-inhibition |
|---|---|---|
| Virus strain | Hemagglutination | using structure (2) at 5 mM |
| A/Aichi/68 (X:31) | ++ | − |
| A/Victoria/3/75 | ++ | − |
| A/Japan/305/57 | ++ | ++ |
| A/Hong Kong/8/68 | ++ | − |
| A/PR/8/34 | ++ | + |
| B/Lee/40 | ++ | − |

As can be seen the A/Japan/305/57 and A/PR/8/34 strains are not inhibited by structure (B) whereas the other strains are completely inhibitable. A sequence comparison between these strains reveals interesting mutations at critical positions which further substantiates the proposed structure of this complex. First of all, any mutations around the primary site are expected to affect hemagglutination and hemagglutination-inhibition equally whereas mutations occurring further along the oligosaccharide chain towards or in the secondary site are expected to affect the hemagglutination-inhibition only. Secondly, mutations at various positions in strains which are completely inhibitable can be discarded as being important for binding. With this line of reasoning at least three mutations at positions 100, 102 and 209 could be identified in both strain A/Japan/305/57 and strain A/PR/8/34 relative to A/Aichi/68 (X:31) and which are localized around the terminal NeuAcα3 in the deepest part of the secondary binding site. The first two mutations are sterically compensatory in nature (Y100G and V102F, identical for both strains) while the third mutation (S209L in A/Japan/305/57 and S209Y in A/PR/8/34) introduces an even more hydrophobic environment than before. Especially the V102F mutation is expected to affect binding strongly since the phenylalanine side chain would come in contact with the sialic acid carboxyl group in the present model

The sequence analysis was carried further by scanning the SwissProt and TREMBL data bases for the 100 most homologous sequences relative to A/Aichi/68 (X:31). By indicating all mutations occurring in these strains by color one gets a view of where on the surface of the hemagglutinin the antigenic drift has been most prevalent in order for the virus to elude the host immune response, and even though it is likely that several of these species-specific strains have different binding specificities the invariant or conservatively mutated regions on the hemagglutinin surface can be regarded as good candidates for ligand interactions. Below three different views of the oligosaccharide binding region is shown with and without the oligosaccharide.

The panels, FIG. 5, shows a “front” view while the panels in FIG. 4 and in FIG. 3 show “right side” and “top” views, respectively. Mutations are colored red and the N-linked sugars are in white whereas the oligosaccharide is shown in yellow. It is evident that the highest mutational frequencies are found on the protruding parts of the protein surface which also are the ones most readily accessible for antibody interactions. The primary site is mainly blue and thus highly conserved as expected as is the path halfway down to the secondary site. However, most of the mutations seen at positions to the lower left of the oligosaccharide point away from the sugars and the mutations to the lower right of the sugars in most cases are conservative or otherwise nondestructive with regard to the secondary binding site topology.

The Complex Structure and Interactions of Oligosaccharide Ligand with the Influenza Virus

Table 1 shows the interactions of the primary site with the saccharide A (oligosaccharide structure 7 according to the Table 3) in complex structure show in FIG. 2. The primary site is referred as Region A, the bridging site referred as region B and the secondary site is referred as Region C. The conserved amino acid having interactions with the oligosaccharide structures are especially preferred according to the invention. The data contains also some semiconservative structures which may mutate to similar structures and even some nonconserved amino acid structures. The nonconserved amino acids may be redundant because their side chains are pointing to the opposite direction. Mutations of the non-conserved or semiconserved amino acid residues are not expected to essentially chance the structure of the large binding site. VDW refers to Van Der Waals-interaction, hb to hydrogen bond. The Table 1 also includes some interactions between amino acid residues in the binding site.

The Table 2 shows the torsion angles between the monosaccharide residues according to the FIG. 1. Glycosidic dihedral angles are defined as follows: phi=H1-C1-O1-C′X and psi=C1-O1-C′X-HX for 2-, 3- or 4-linked residues; phi=H1-C1-O1-C′6, psi=C1-O1-C′6-C′5 and omega=O1-C′6-C′5-O′5 for a 6-linked residue. Imberty, A., Delage, M.-M., Bourne, Y., Cambillau, C. and Pérez, S. (1991) Data bank of three-dimensional structures of disaccharides: Part II. N-acetyllactosaminic type N-glycans. Comparison with the crystal structure of a biantennary octasaccharide. Glycoconj. J., 8, 456-483. The torsion angles define conformation of oligosaccharide part in the complex structure.

### Synthesis of the Branched Oligosaccharide Library

### Starting Materials for Enzymatic Synthesis

LNnT (LNβ3L) and Gnβ3LNβ3L were from commercial sources or enzymatically synthesized. Gnβ6L was purchased from Sigma (USA). LNH [LNβ3(Gβ3Gnβ6)L] was purchased from Dextra Laboratories (UK).

### Enzymatic Synthesis

### α1,3-Galactosyltransferase (GalT3)

5 mM acceptor and 10 mM UDP-galactose were incubated with GalT3 (0.02 mU of enzyme/nmol of acceptor site was used; recombinant enzyme from bovine, Calbiochem, cat. no. 345647) in 0.1 M MES, pH 6.5 and 20 mM MgCl₂ for 24 hours at 37° C. Reaction was terminated by incubation on a boiling water bath for 3 min.

### β1,6-N-acetylglucosaminetransferase (GnT6)

8 mM acceptor and molar excess of UDP-N-acetylglucosamine were incubated with GnT6 (as enzyme source fresh, concentrated rat serum was used) in 20 mM EDTA, 0.5 mM ATP, 200 mM Gal, 60 mM γ-galactonolactone, 8 mM NaN₃ for 7 days at 37° C. Reaction was terminated by incubation on a boiling water bath for 3 min.

### β1,4-Galactosyltransferase (GalT4)

4 mM acceptor and 8 mM UDP-galactose were incubated with GalT4 (0.05 mU of enzyme/nmol of acceptor was used; enzyme from bovine milk, Calbiochem, cat. no. 345649) in 50 mM MOPS pH 7.4 and 20 mM MnCl₂ for 6 hours at 37° C. Reaction was terminated by incubation on a boiling water bath for 3 min.

### β1,3-N-acetylglucosaminetransferase (GnT3)

2 mM acceptor and molar excess of UDP-N-acetylglucosamine were incubated with GnT3-preparate (see below) in 0.1 mM ATP, 0.04% NaN₃ and 8 mM MnCl₂ for five days at 37° C. Reaction was terminated by incubation on a boiling water bath for 3 min. Concentrated human plasma was used as GnT3-preparate: Human plasma was purchased from Finnish Red Cross, a protein concentrate from ammoniumsulphate precipitation of 25-50% was obtained, dissolved to 50 mM Tris-HCL, pH 7.5 and 0.5 M NaCl, dialyzed against highly purified H₂O and lyophilized. Prior to use, enzyme preparate was dissolved to 50 mM Tris-HCL, pH 7.5.

### α2,6-Sialyltransferase (SAT6)

1. Enzyme from ICN

8 mM acceptor and molar excess of CMP-N-acetylneuraminic acid were incubated with SAT6 (3 μU of enzyme/nmol of acceptor site was used) in 50 mM MES, pH 6.0 and 0.2% BSA for 48 hours at 37° C. Reaction was terminated by incubation on a boiling water bath for 3 min.

2. Enzyme from Bovine Colostrum

10 mM acceptor and molar excess of CMP-N-acetylneuraminic acid were incubated with SAT6-preparate (see below) and 0.02% NaN₃ for 72 hours at 37° C. Reaction was terminated by incubation on a boiling water bath for 3 min.

Fat was removed by centrifugation of fresh or at −20° C. stored bovine colostrum. Casein was removed by acid precipitation after which the preparate was neutralized. A protein concentrate from ammoniumsulphate precipitation of 40-60% was obtained, dissolved to 10 mM MES, pH 6.0, dialyzed against the same buffer and lyophilized. Prior to use, enzyme preparate was dissolved to highly purified H₂O.

3. Recombinant Enzyme from Rat, Calbiochem, Cat. No. 566222

8 mM acceptor and molar excess of CMP-N-acetylneuraminic acid were incubated with SAT6 (4 μU of enzyme/nmol of acceptor site was used) in 50 mM MES, pH 6.0, 0.1% Triton-X-100, 0.5 mg/ml BSA and 0.02% NaN₃ for 48 hours at 37° C. Reaction was terminated by incubation on a boiling water bath for 3 min.

### α2,3-Sialyltransferase (SAT3)

4 mM acceptor and 8 mM CMP-N-acetylneuraminic acid were incubated with SAT3 (0.1 U of enzyme/μmol of acceptor site was used; enzyme from rat, recombinant, Calbiochem, cat. no. 566218) in 50 mM MOPS pH 7.4 and 0.2 mg/ml BSA for 48 hours at 37° C. Reaction was terminated by incubation on a boiling water bath for 3 min.

### α1,3-Fucosyltransferase (FucT3)

1 mM acceptor and 5 mM GDP-fucose were incubated with FucT3 (0.02 mU of enzyme/nmol of acceptor was used; enzyme human FucT VI, recombinant, Calbiochem, cat. no. 344323) in 50 mM MOPS pH 7.2 and 20 mM MnCl₂ for 24 hours at 37° C. Reaction was terminated by incubation on a boiling water bath for 3 min.

### Enzymatic Degradation Reactions

α2-3,6,8-Sialidase (SA′ase)

8 mM substrate was incubated with sialidase (13 mU/μmol of substrate; Vibrio cholerae sialidase was from Calbiochem, cat. no. 480717) in 50 mM Na-acetate buffer, pH 5.5 and 1 mM CaCl₂ for 24 hours at 37° C. Reaction was terminated by incubation on a boiling water bath for 3 min.

β1,3-Galactosidase (Gal′ase)

8 mM substrate was incubated with galactosidase (13 mU/μmol of substrate; recombinant enzyme from Calbiochem, cat. no. 345795) in 50 mM Na-citrate buffer, pH 4.5 for 24 hours at 37° C. Reaction was terminated by incubation on a boiling water bath for 3 min.

### Synthesis of the Branched Oligosaccharide Library

The oligosaccharide library represented in Table 3 was synthesised using the preferred methods according to the invention and as described for example by the schemes 1-6. The oligosaccharides were purified using chromatographic methods and the products were characterized by MALDI-TOF mass spectrometry and NMR-spectroscopy.

### Preparation of Divalent Conjugates

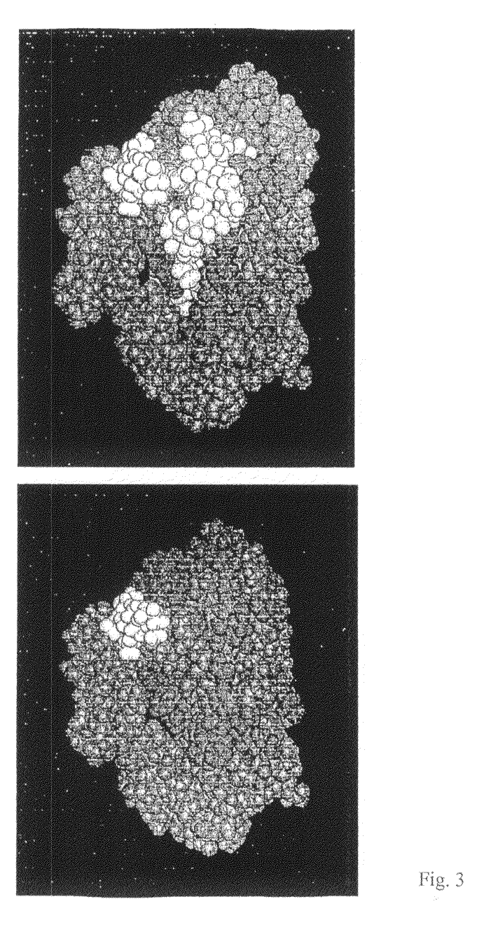

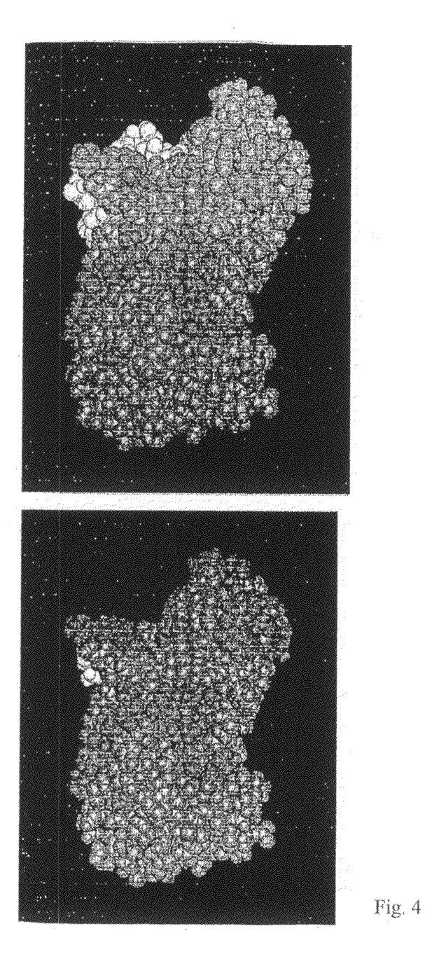

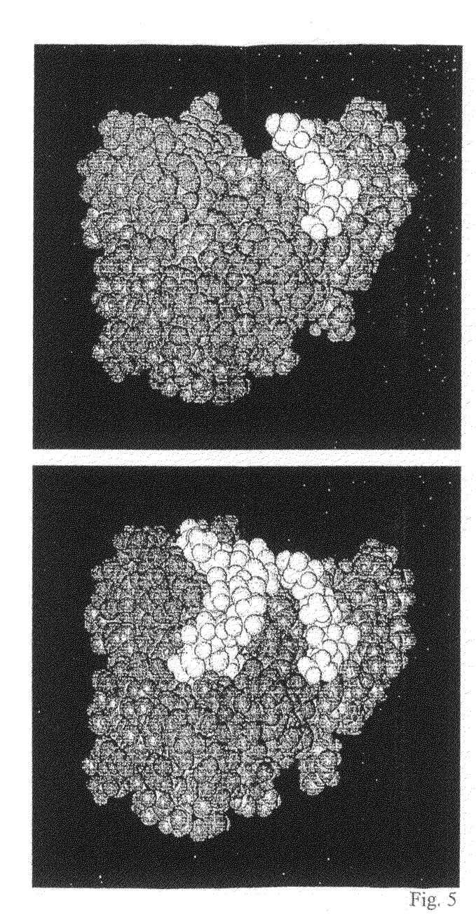

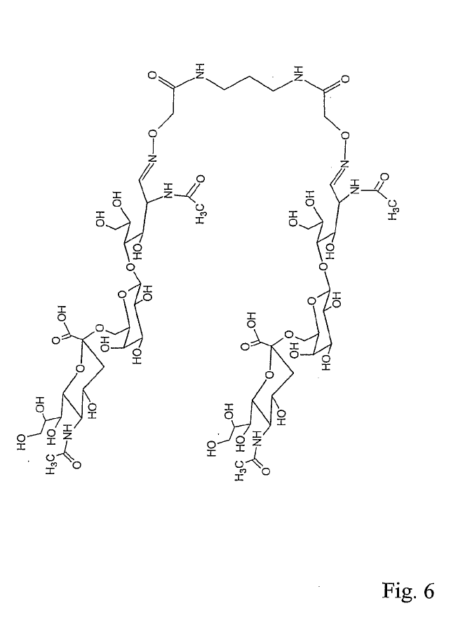

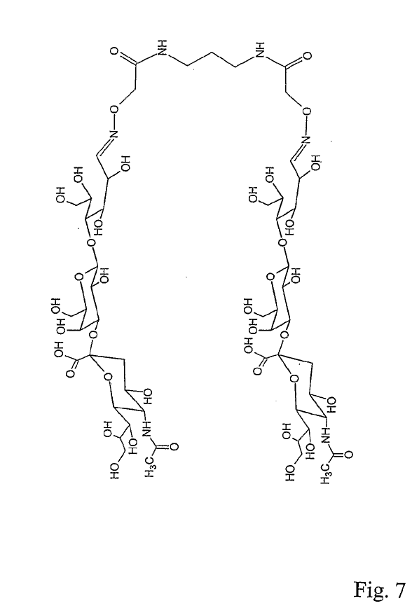

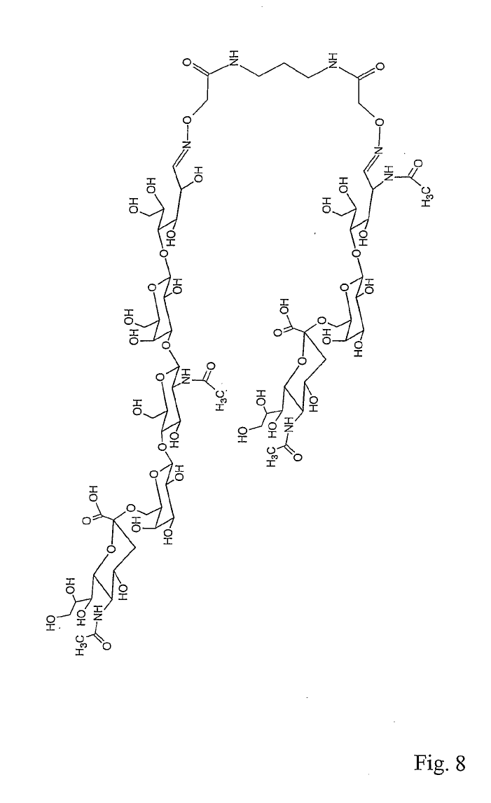

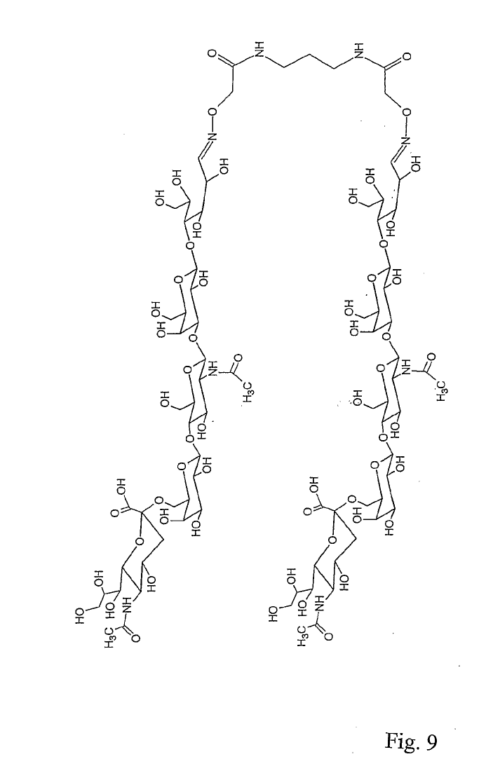

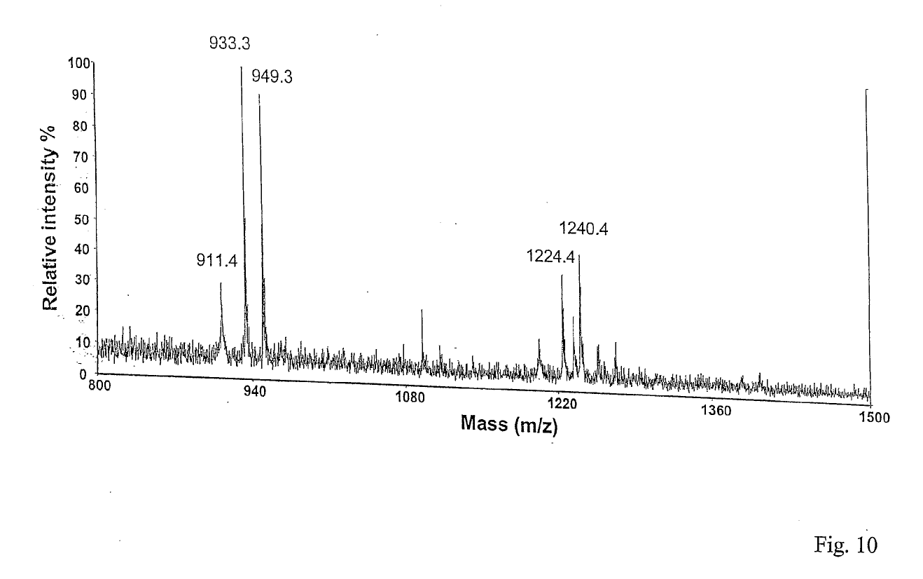

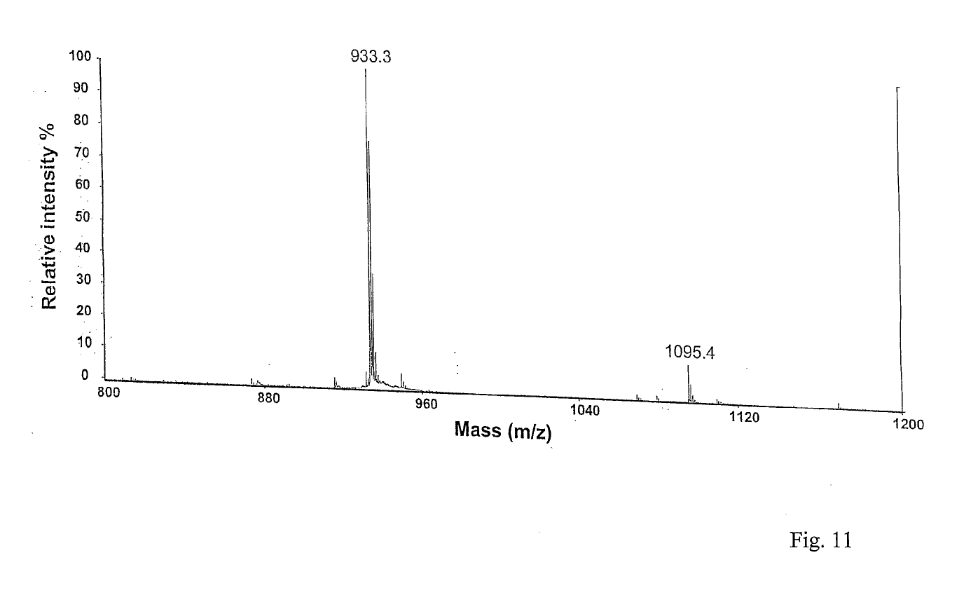

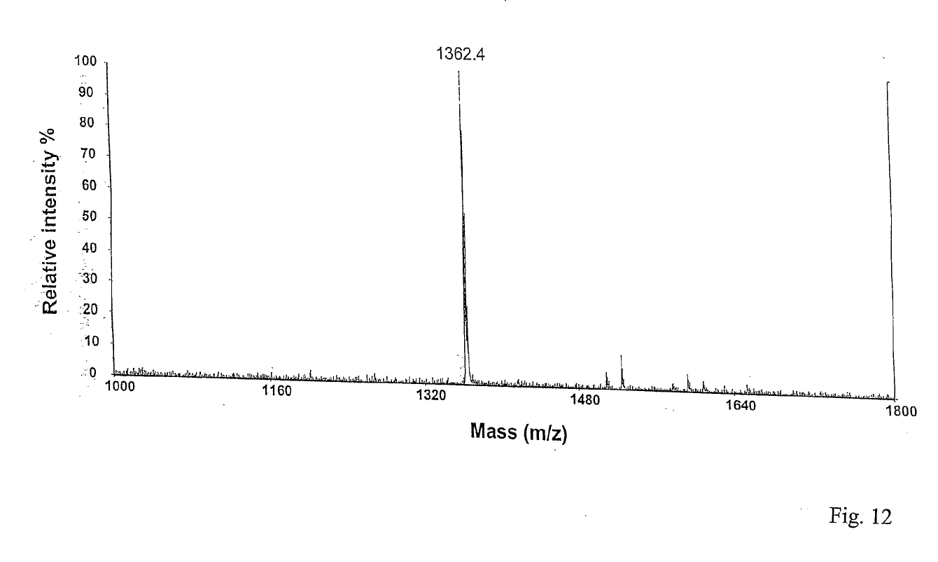

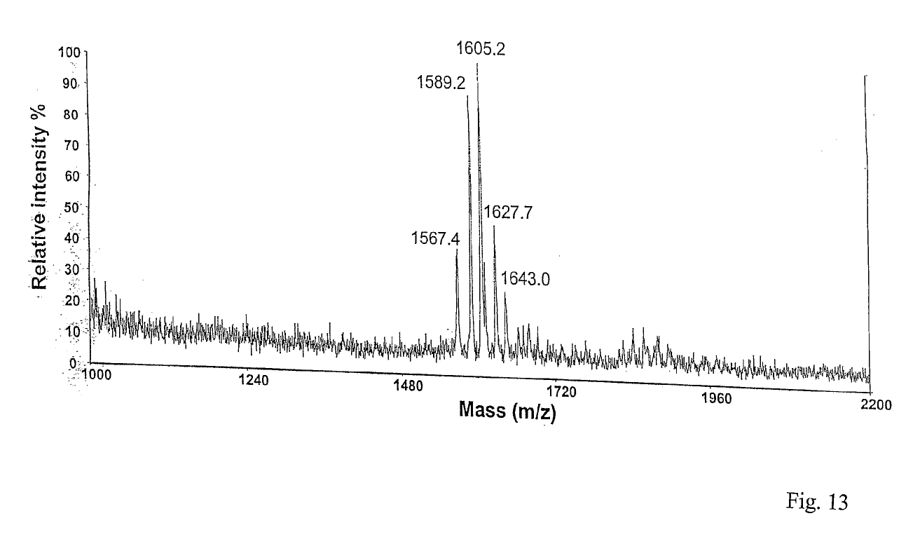

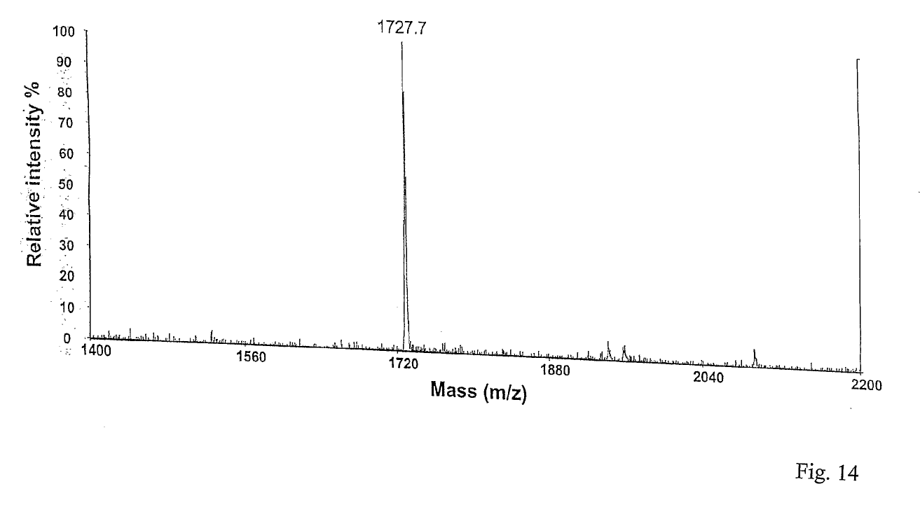

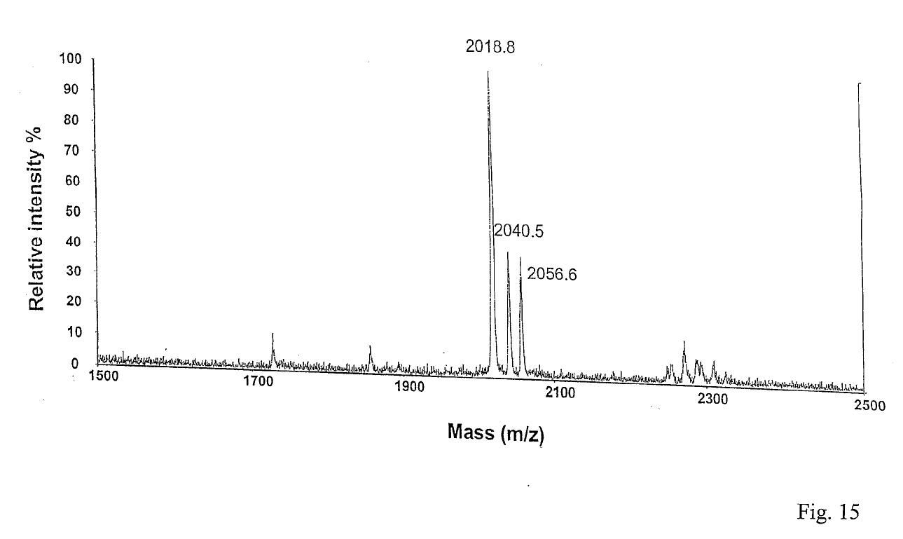

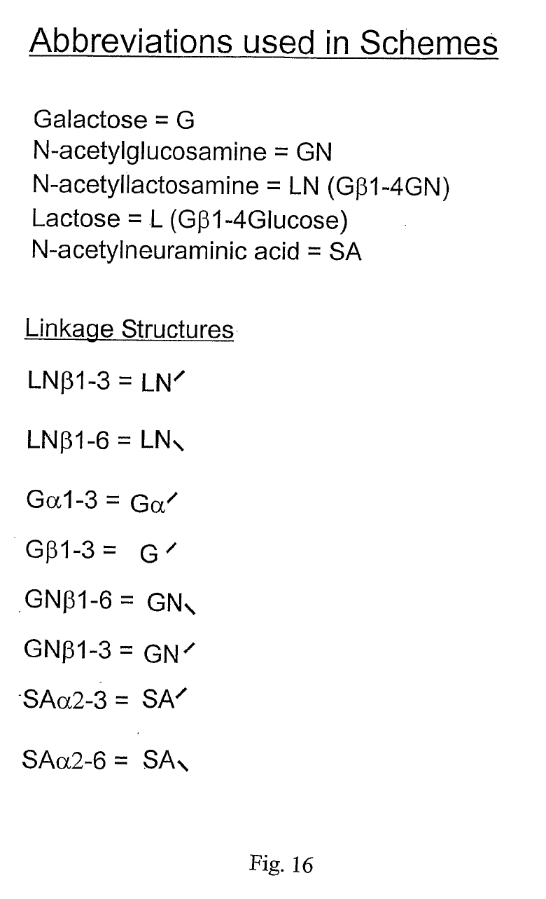

A divalent aminooxy reagent (N,N′-diaminooxyacetic acid amide of 1,3-diaminopropane, DADA) was used to produce divalent carbohydrate molecules through oxime formation. One micromole of DADA was incubated with 5 micromoles of reducing carbohydrate in 0.2 M sodium acetate buffer, pH 4.0, for 42 h at 37° C. The divalent carbohydrate oxime was purified with gel-permeation chromatography, and subjected to NMR spectroscopic analysis. The NMR data confirmed the formation of hydroxylamine-glycosidic bond, but it is also clear that about 50% of the reducing sugar exists in pyranose form FIGS. 6, 7, 8 and 9 represent divalent conjugates of two Neu5Acα6LacNAc, of two Neu5Acα3Lac, of one Neu5Acα6LacNAc and one Neu5Acα6LacNAcβ3Lac, and of two Neu5Acα6LacNAcβ3Lac, respectively. The ¹H-NMR spectrum of DADA conjugates were analysed and special characteristic signals were observed such as signals generated when the glucose is in a non-pyranose or linear form in the oxime and signal generated when glucose is in a pyranose ring form, and signals of the oligosaccharides and the spacers were observed.

### MALDI-TOF Mass Spectrometry

MALDI-TOF mass spectra were collected using an Applied Biosystems Voyager STR mass spectrometer in delayed extraction mode, using nitrogen laser.

Spectra in the positive ion mode were acquired using 2,5-dihydroxybenzoic acid (DHB, 10 milligrams/milliliter in deionised water) as the matrix. Samples were dissolved in water to a concentration of 1-10 pmol/microliter, and one microliter of sample was mixed with one microliter of matrix, and dried with a gentle stream of air to the stainless steel target plate. Typically 50-200 shots were summed for the final spectrum. The spectra were externally calibrated with maltooligosaccharide mixture.

Spectra in the negative ion mode were acquired using trihydroxyacetophenone (THAP, 3 milligrams/milliliter in 10 mM diammonium citrate/acetonitrile, 1:1) as the matrix. Samples were dissolved in water to a concentration of 1-10 pmol/microliter, and 0.3 microliters of sample solution was deposited to the target plate, followed by 0.3 microliter of matrix solution. The droplet was immediately dried under reduced pressure to produce a thin uniform sample spot. Prior to mass spectrometric analysis, the spot was allowed to absorb moisture until clearly white or gray. Typically 50-200 shots were summed for the final spectrum. The spectra were externally calibrated with a mixture of established sialylated oligosaccharides prepared in the laboratory.

### NMR-Spectroscopy of the Branched Oligosaccharide Library

NMR spectroscopy was performed in D2O at 23 degrees of Celsius using a 500 MHz NMR-spectrometer.

Following structural reporter group data were used in defining the structures. The primary structure of lactosaminoglycans can be determined from one dimensional NMR spectra. Structural elements are identified from signals having characteristic chemical shifts. The integration of these signals gives the relative amount of different types of monosaccharides within the glycan. Typical structure reporting signals are the anomeric H1 protons and other protons at or near the site of glycosidic linkage. The anomericity of the monosaccharides is obtained from the H1-H2 coupling constant. Typical values are 3-4 Hz for α anomer and 7-8 Hz for β anomer. In branched lactosaminoglycans H1 protons of Gal and GlcNAc have distinct chemical shift depending on whether Gal is 3- or 6-substituted of if Gal is both 3- and 6-substituted. In repeating 4GlcNAcβ1-3Galβ1 structures Gal H1 and GlcNAc H1 resonate at 4.46 ppm and 4.70 ppm, respectively. Another characteristic feature of GlcNAcβ1-3Gal structures is the Gal H4 signal, which resonates at 4.16 ppm. In structures having Galβ1-4GlcNAcβ1-6Gal the chemical shift of GlcNAc H1 is ˜4.63 ppm. The terminal Gal H1 has different chemical shifts depending on whether it is in the 3- or 6-branch. The chemical shifts are 4.48 ppm and 4.46 ppm, respectively. A GlcNAc in all structures is also identified from the methyl signal of the N-acetyl group between 2.02-2.07 ppm. Sialylated lactosaminoglycans have also easily recognizable signals. The linkage isomers e.g. Neu5Acα2-3/6Gal can be distinguished. In Neu5Acα2-3Gal the equatorial and axial H3 of Neu5Ac resonate at 2.76 ppm and 1.80 ppm, respectively. The Gal H3 signal is observed at 4.12 ppm and Gal H1 resonates at 4.56 ppm. In Neu5Acα2-6Gal the equatorial and axial H3 of Neu5Ac resonate at 2.67 ppm and 1.72 ppm, respectively. The Gal H3 in buried under the signals of other skeletal protons and cannot be assigned from the one dimensional spectrum. In both isomeric forms the methyl signal of the N-acetyl group is observed at approximately 2.06 ppm. More specific shifts were used in defining the key branched precursor structures.

### Hemagglutination Inhibition Studies

In the hemagglutination inhibition studies Influenza viruses were incubated at room temperature for one hour in mixture containing 25 microliters Influenza virus (about 8 Hemagglutination units), 10 microliters buffered inhibitor solution in various concentrations, and 25 microliters erythrocytes. Hemagglutination inhibition was determined as lowest microscopically detectable inhibitory concentration.

Results with large oligosaccharide in hemagglutination inhibition with A/Victoria/3/5

| Oligosaccharide no | Relative effectivity |
|---|---|
| 7 | 66 |
| 16 | 26 |
| 17 | 15 |
| 18 | 8 |
| 19 | <5 |
| 20 | 7.5 |
| 21 | 100 |
| 20 | 19 |
| 1-4 | <2 |

The data indicates that the saccharide 7 modelled on the influenza virus surface is the most active isomer of the branching and sialylation isomeric decasaccharide structures. The data also shows that the doubly α6-sialylated 12-meric saccharide was even more effective. The data further indicates that even α3-sialylated branched polylactosamine structures have reasonable activity in inhibiting hemagglutination.

### Hemagglutination Inhibition by Spacer Linked Divalent Saccharides

| Virus strain | | | | |
|---|---|---|---|---|
| | | | A/Hong | |
| A/Victoria | A/X:31- | A2/Japan | Kong | |
| 3/73, H3N2 | Aichi, H3N2 | 305/57 | 8/68 H3N2 | B/Lee |

Inhibitor

| | | | | | |
|---|---|---|---|---|---|
| None | ++ | ++ | ++ | ++ | ++ |
| Fetuin | − | − | − | − | − |

Oligosaccharide

| | | | | | |
|---|---|---|---|---|---|
| 25 | − | − | ++ | − | ++ |
| 26 | + | ++ | + | ++ | − |
| 27 | − | − | ++ | +/− | ++ |
| 28 | − | − | ++ | +/− | ++ |

The oligosaccharides were tested under conditions as above wherein monovalent epitopes are virtually inactive. The linking of the monovalent structures to divalent ones increases the effectivity of the structures. The divalent structure 25 had an activity comparable with the best polylactosamine structures described above. The data further shows that α3-sialylated simple epitope has some effectivity against α3-sialylated specific strains. The A/Victoria and B/Lee strains were from Charles Rivers Laboratories USA, and origin of the rest of the strains is as described below.

The Effectivity of α3-Sialylated Structures with Several Strains on a Solid Phase Assay

The data in Table 4 shows binding of various influenza strains to branched oligosaccharide structures 8 and 9 when the oligosaccharides are reductively aminated (by cyanogen borohydride) to lipid carrier structure: Lysine-dipalmitate-amide bonded to diaminepropane (C42) or phosphadityethanolamine. The data shows that many new and old influenza strains bind effectively to sialylated complex gangliosides (likely polylactosamines) of human granulocytes. Interestingly almost all strains bind to both types of conjugates with some strains being specific for α3- and some for α6-linked sialic acids. The binding of the conjugates, also α3-linked conjugates was effective and very reproducible. In comparison to monosialylated non-branched glycolipid Neu5Acα3Galβ4GlcNAcβ3LacβCer, the binding was very reproducible and estimated to be at least about order of magnitude more effective. The following viruses of Table 4 were from Hytest, Turku, Finland: A/Taiwan; A/Beijing, A/New Caledonia, A/Kiew, A/Shangdong; the following strains of Table 4 were from Charles Rivers Laboratories (USA): A/PR, A/X31, A/2, A/Hong Kong.

### Additional Modelling Work

Distances between carboxylic acid groups of sialic acid residues in binding conformation were produced with X31-hemagglutinin model. The large divalent saccharide 25 with two α6-sialylpentasaccharides had an extended length (most likely conformation with regard to glycosidic torsion angles) of about 59 Å and it could be docked to the primary and secondary sites, the saccharide 26 had an extended length of 47 Å and it could not be docked both to primary and secondary site, the saccharide 27 had extended length of 36 Å and could be fitted to both primary and secondary sites with a configuration similar to saccharide 17; and the saccharide 28 has the extended length of 49 Å with docking to both primary and secondary site.

### Example 2

### Materials and Methods for ELISA Assays of Peptides

### ELISA Assays on Maleimide-Activated Plates

Peptides containing cysteine were bound through the cysteine sulfhydryl group to maleimide activated plates (Reacti-Bind™ Maleimide activated plates, Pierce). The peptides sequences were as follows:
Biotin-aminohexanoyl-SYACKR (custom product, CSS, Edinburgh, Scotland)
Biotin-aminohexanoyl-SKAYSNC (custom product, CSS, Edinburgh, Scotland)
CYPYDVPDYA (HA11; Nordic Biosite)

All peptides were dissolved in 10 mM sodium phosphate/0.15 M NaCl/2 mM EDTA, pH 7.2, to a concentration of 5 nmol/ml. One hundred microliters of the peptide solution (0.5 nmol of peptide) was added to each well and allowed to react overnight at +4° C. The plate was then washed three times with 10 mM sodium phosphate/0.15 M NaCl/0.05% Tween-20, pH 7.2).

The unreacted maleimide groups were blocked with 2-mercaptoethanol: 150 μl of 1 mM 2-mercaptoethanol in 10 mM sodium phosphate/0.15 M NaCl/2 mM EDTA, pH 7.2 was added to each well and allowed to react for 1 hour at RT. The plate was then washed three times with 10 mM sodium phosphate/0.15 M NaCl/0.05% Tween-20, pH 7.2. The plate was further blocked with 1% bovine serum albumin (BSA) in 10 mM sodium phosphate/0.15 M NaCl/0.05% Tween-20, pH 7.2, and then washed with 10 mM sodium phosphate/0.15 M NaCl/0.05% Tween-20/0.2% BSA, pH 7.2 (washing buffer).

Serum was obtained from six healthy individuals (29-44 years of age), and dilutions 1:10, 1:100 and 1:1000 were prepared from all but one serum sample in the washing buffer. The serum obtained from person nr. 5 was instead diluted 1:25, 1:250 and 1:2500 in the washing buffer. One hundred microliters of each serum sample was added to the wells and incubated for 30 mins at RT. Control wells contained no peptide but both 2-mercaptoethanol and BSA blockings were employed. All incubations were performed in duplicates.

The plate was then washed with the washing buffer 8 times with at least 5 min incubation period between change of the washing liquid.

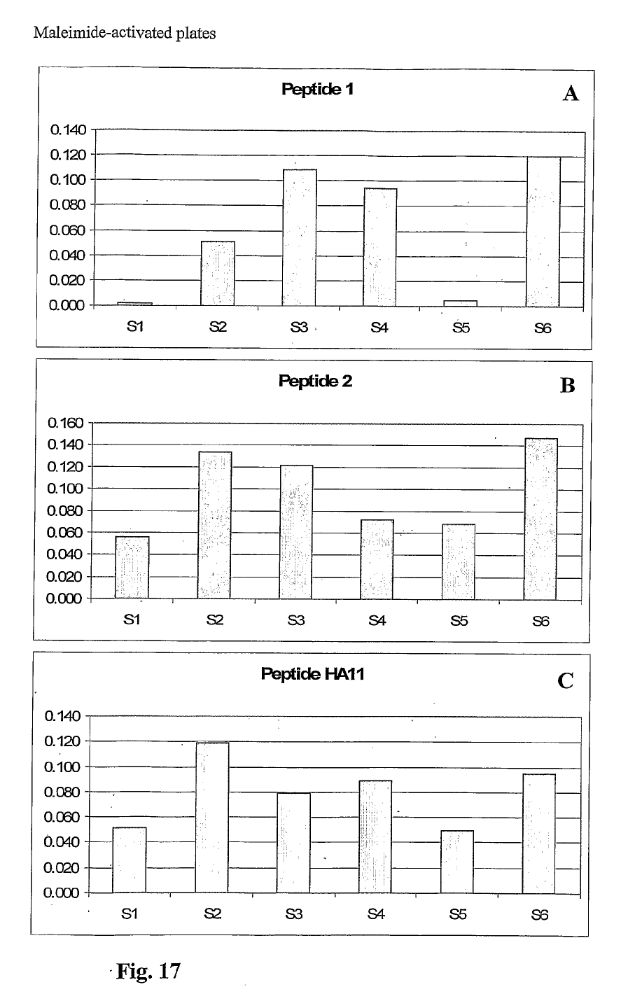

The bound serum antibodies were quantitated by adding anti-human IgG (rabbit)—HRP conjugate (Sigma) in 1:30000 dilution to each well. After one hour incubation at RT, the plate was washed five times with the washing buffer. One hundred microliters of TMB+ color reagent (Dako Cytomation) was then added. The absorbance was read at 650 nm after 15 mins. Immediately after this measurement 100 μl of 1 M sulphuric acid was added and the absorbance read at 450 nm. Results are shown in FIG. 17.

### ELISA Assays on Streptavidin-Coated Plates

Biotinylated peptides were bound to streptavidin-coated plates (Pierce).

The peptides sequences were as follows:
Biotin-aminohexanoyl-PWVRGV (custom product, CSS, Edinburgh, Scotland)
Biotin-aminohexanoyl-SYACKR (custom product, CSS, Edinburgh, Scotland)
Biotin-aminohexanoyl-SKAYSNC (custom product, CSS, Edinburgh, Scotland)

Prior to peptide immobilization, plates were blocked with 150 μl of 0.5% BSA in 10 mM sodium phosphate/0.15 M NaCl/0.05% Tween-20, pH 7.2, for 1.5 h at RT. The plate was then washed three times with 10 mM sodium phosphate/0.15 M NaCl/0.05% Tween-20, pH 7.2.

Peptides were dissolved in 10 mM sodium phosphate/0.15 M NaCl, pH 7.2, to a concentration of 0.5 nmol/ml. One hundred microliters of the peptide solutions (50 pmol of the peptide) were added to the wells and allowed to react overnight at +4° C. The plates were then washed four times with 10 mM sodium phosphate/0.15 M NaCl/0.05% Tween-20/0.2% BSA, pH 7.2 (washing buffer).

Serum was obtained from six healthy individuals (29-44 years of age), and dilutions 1:10, 1:100 and 1:1000 were prepared from all but one serum sample in the washing buffer. The serum obtained from person nr. 5 was instead diluted 1:25, 1:250 and 1:2500 in the washing buffer. One hundred microliters of each serum sample was added to the wells and incubated for 60 mins at RT. Control wells did not contain peptides but were blocked as above. All incubations were performed in duplicates.

After serum incubation the plate was washed with the washing buffer 8 times with at least 5 min incubation period between change of the washing liquid.

The bound serum antibodies were quantitated by adding anti-human IgG (rabbit)—HRP conjugate (Sigma) in 1:30000 dilution to each well. After one hour incubation at RT, the plate was washed five times with the washing buffer. One hundred microliters of TMB+ color reagent (Dako Cytomation) was then added. The absorbance was read at 650 nm after 15 mins. Immediately after this measurement 100 μl of 1 M sulphuric acid was added and the absorbance read at 450 nm.

### Results of ELISA Assays of Antigen Peptides

### Design of the Experiments

Three antigen peptides were analysed against natural human antibodies from healthy adults. The individuals were selected based on the resistance against influenza for several years. The persons had been in close contact with persons with distinct influenza type disease in their families and/or at work but have not been infected for several years. At the time of blood testing two of the persons had influenza type disease at home but persons were suffering from only mild disease. The persons were considered to have good immune defense against current influenza strains.

The antigen peptides were selected to correspond structures present on recent influenza A (H3N2) strains in Finland (home country of the test persons). The assumption was that the persons had been exposed to this type of viruses and they would have antibodies against the peptides, in case the peptides would be as short linear epitopes effectively recognizable by human antibodies and peptide epitopes would be antigenic in human. The invention revealed natural human antibodies against each of the peptides studied. The data indicates that the peptides are antigenic and natural antibodies can recognize effectively such short peptide epitopes.

All antigen peptides 1-3 were tested as N-terminal biotin-spacer conjugates, which were immobilized on a streptavidin plate. Aminohexanoic acid spacer was used to allow recognition of the peptides without steric hindrance from protein. It is realized that the movement of the N-terminal part of peptide was limited, which would give conformational rigidity to the peptide partially mimicking the presence on a polypeptide chain.

The Peptides 1 and 2 were also Tested on Maleimide Coated Plates.

The peptide 1 (Biotin-aminohexanoic-SKAYSNC) was also tested as conjugated from natural C-terminal Cys-residue in a antigen peptide, the peptide further contained spacer-biotin structure at amino terminal end of the peptide. The peptide presented natural C-terminal and Cys-linked presentation at C-terminus of the peptide presenting a preferred conformational structure. The presentation as natural like epitope was further supported by spacer structure blocking the N-terminus and restricting its mobility.

The peptide 2 (Biotin-aminohexanoic-SYACKR) was also tested as conjugated from natural Cys-residue in the middle of the antigen peptide. The peptide presented natural middle Cys-linked presentation at C-terminus of the peptide presenting a preferred conformational structure. The presentation as natural like epitope was further supported by spacer structure blocking the N-terminus and restricting its mobility.

### Control and Core Peptide

A commercial peptide CYPYDVPDYA (HA11-peptide), which has been used as a recognition tag on recombinant proteins was used as a control and for testing of analysis of binding between a free core peptide and human antibodies. Due to restricted availability of at least N-terminal sequence the peptide would not be very effective in immunization against the viral as therapy. This peptide is known to be antigenic in animals under immunization conditions and antibodies including polyclonals from rabbit, mice etc. The ELISA assay was controlled by effective binding of commercial polyclonal antibody from rabbit to the peptide coated on a maleimide plate, while negligible binding was observed without the peptide.

### Results

The absorbance was recorded by two methods (A450 and A650) and with three different dilutions giving similar results (the results with optimal dilutions giving absorbance values about 0.1 AU to about 0.8 AU and by absorbance at 450 nm are shown).

### Peptide 1 as Aminoterminal Conjugate and C-Terminal Cys-Conjugate

Biotin-aminohexanoic-SKAYSNC was tested against the 6 sera as N-terminal conjugate on a streptavidin plate. The sera 3 and 4 showed strongest immune response before serum 2, while sera 1, 5 and 6 were weakly or non-reactive against the construct.

The C-terminal cysteine conjugate of peptide 1 reacted with sera in the order from strongest to weaker: 6, 3, 4, and 2, while 1 and 5 were weakly or non-reactive against the construct. The results indicated, that both conjugates reacted remarkably similarly with antibodies except the serum 6 which contained antibodies preferring the structure including the immobilized cysteine as in natural peptides on viral surface.

### Peptide 2 as Aminoterminal Conjugate and Middle Cys-Conjugate

Biotin-aminohexanoic-SYACKR was tested against the 6 sera as N-terminal conjugate on a streptavidin plate. The sera 2 and 5 showed strongest immune response before sera 3,4 and 6,while serum 1 showed weakest reaction.

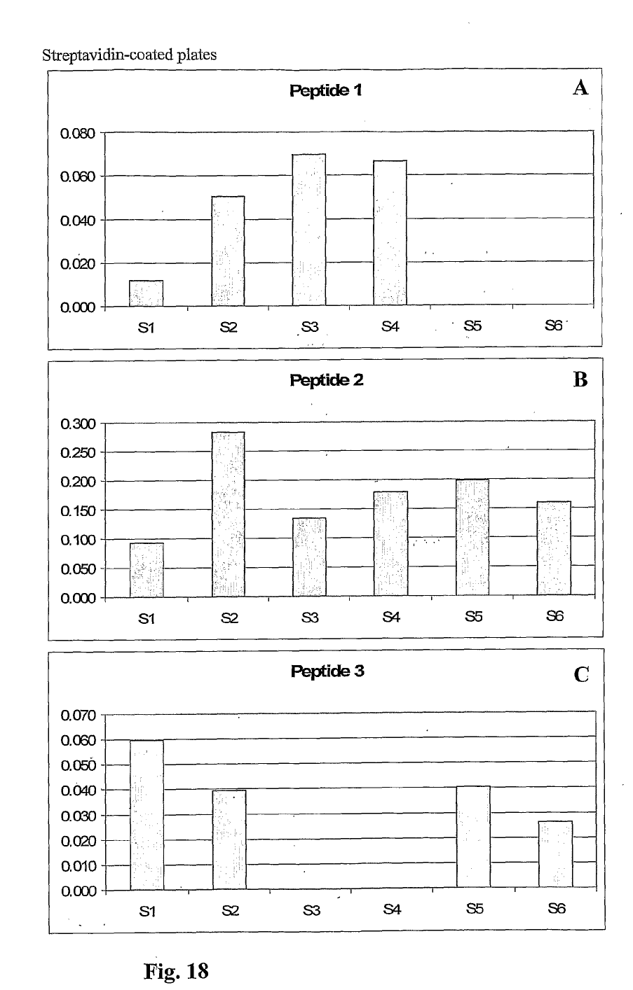

The middle cysteine conjugate of peptide 2 reacted with sera similarity but reactions with serum 5 was weaker and the serum 6 showed the strongest response, see FIG. 18 and Table 5. The results indicated, that both conjugates reacted remarkably similarly with antibodies except the serum 6 which contained antibodies preferring the structure including the immobilized cysteine as in natural peptides on viral surface.

### Peptide 3

Peptide 3 has distinct pattern of immune recognition as shown in Table 5.

Correlation of the Immune Reaction with Viral Presentation of the Peptides 1-3 and HA11

More than hundred recently cloned human influenza A viruses were studied with regard to presentation of peptides 1-3. It was realized that there is one to a few relatively common escape mutants of each one of these, which would be different in antigenicity in comparison to the peptides 1-3. The analysis further reveled that on average the viruses contain two of the peptides 1-3. Thus the result that each influenza resistant test subject had antiserum at least against two of peptides fits well data about the recent viruses in Finland. The data further support the invention about combination of the antigenic peptides. The combination of at least two peptides is preferred.

The control core sequence HA11 is present as very conserved sequence in most influenza A viruses and thus all persons would have been immunized against it as shown by the results in Table 5.

### Example 3

### Analysis of Conserved Peptide Epitopes 1-3 in Hemagglutinins H1, H2, and H3

The presence of hemagglutinin peptide epitopes 1-3 were analysed from hemagglutinin sequences. Tables 6 and 7 shows presence of Peptides 1-3 in H1 hemagglutinins as typical H1 Peptide 1-3 sequences. The analysis revealed further sequences, which are conserved well within H1 hemagglutinins. These are named as PrePept1-4 and PostPept1-4. These conserved aminoacid sequences are preferred for sequence analysis and typing of influenza viruses. The PrePept1-3 and PostPept1-4 sequences were found to be characteristics for H1, with partial conservation of amino acid residue. The PrePept4 in its two forms WGVHHP and more rarely homologous WGIHHP were revealed to be very conserved among all A-influenza viruses.

Table 8 shows Peptide 1-3 sequences from selected H2 viruses. Characteristic sequences for H2-type influenza viruses were revealed.

Table 9 shows analysis Peptides 1-4 from large group recent human influenza viruses containing H3 hemagglutinins. Several homologous sequences for each peptides 1-3 were revealed.

When comparing with data of serum Elisa experiment (see Example 2) a correlation was revealed. In most of the strains only one peptide epitope is likely mutated in the virus, which had immunized the persons, in comparison to peptides selected for the assay. As the immune defense had been likely obtained during 80′ and/or 90′ as the persons have not had several influenza during recent years, the recent variants of peptide 1 and 2 were likely not causing the antibody production, which might have been yielded less pronounced reaction against the peptides 1-3 used in the ELISA experiment. The non-reactivity against peptide 1 may have been caused by X31 type SKAFSN-immunization during earlier decades when this type of sequence would have more frequent, but the antibodies would be less reactive with the hydrophilic variant of SKAYSN used in the experiments.

The invention is further directed to the use of the conserved PrePept and Post Pept sequences for analysis of corresponding Peptide 1-4 sequences. The conserved sequences may be used for example as targets of specific protease sequencing reagents of nucleic acid sequencing reagents such as RT-PCR primers. The peptide 1 can effectively sequences by using closely similar PrePept1 and PostPept1 sequences or other PostPept sequences (which would also yield other Peptide 2, 3 and/or 4 sequences depending on the selection of PostPeptide).

The invention is further directed to analysis of the carbohydrate binding status and/or infectivity of an influenza virus by analysing the sequence of Peptides 1-3 and/or Peptide 4. The invention is directed to the analysis by sequencing the protein and/or corresponding nucleic acids or by recognizing the peptides by specific antibodies, preferably by specific human antibodies.

TABLE 1
Summary of interactions between hemagglutinin X31 Aichi and saccharide 7

Interactions

REGION A

| | |
|---|---|
| Conserved a.a.* | |
| Tyr98 | Hb between Tyr OH and Siaα6 O9 |
| Gly135 | Hydrophobic patch: Gly —CH₂ and Siaα6 acetamido —CH₃ |
| Ser136 | Hb between Ser OH and Siaα6 ⁻OOC— |
| Trp153 | Hydrophobic patch: Trp indole and Siaα6 acetamido —CH₃ |
| His183 | Hb between His NH and Siaα6 O9 |
| Leu194 | VDW packing |
| Gly225 | Hairpin loop |
| Semi- or nonconserved a.a.* | |
| Gly134 | VDW packing |
| Asn137 | Hb between Asn NH and Siaα6 ⁻OOC—(long) |
| Ala138 | Hydrophobic patch: Ala —CH₃ and Leu226 —CH₃ |
| Thr155 | Hydrophobic patch: Thr —CH₃ and Trp153 indole |
| Glu190 | Hb between Glu COO⁻and Siaα6 OH9 |
| Leu226 | VDW packing (see also Ala138) |

REGION B

| | |
|---|---|
| Conserved a.a.* | |
| Ser95 | Hb between Ser OH and Asp68 ⁻COO— |
| Val223 | VDW packing |
| Arg224 | Hydrophobic patch: Arg —CH₂—CH₂— and hydrophobic |
| | side of GlcNAcβ6 |
| Gly225 | Hairpin loop |
| Trp222** | Hydrophobic patch: Trp indole and hydrophobic side of |
| | Manα4GlcNAc of glycan linked to Asn165 |
| Asn165-linked glycan | Possible interactions with saccharide 7 (only first three |
| | glycan sugars are visible by X-ray |
| Semi- or nonconserved a.a.* | |
| Phe 94 | VDW packing |
| Asn96 | Hb between Asn amido C═O and GlcNAcβ6 O3 |
| Asn137 | Hb between Asn amido C═O and GlcNAcβ3 O6 |
| | (short arm) |
| Ala138 | Hydrophobic patch: Ala —CH₃ and Leu226 —CH₃ |
| Lys140 | Hydrophobic and electrostatic interactions with Glcβ |
| Arg207 | Hb between Arg guanidino NH and GlcNAcβ3 O4, |
| | VDW packing |

REGION C

| | |
|---|---|
| Conserved a.a* | |
| Thr65 | Hb between Thr OH and Siaα6 ⁻OOC— |
| Ser71 | Hb between Ser OH and Siaα6 4OH |
| Glu72 | Salt bridge with Arg208 |
| Ser95 | Hb between Ser OH and Asp68 ⁻OOC— |
| Gly98 | Protein fold |
| Pro99 | Protein fold |
| Tyr100 | Hb between tyr OH and Galβ O4 |
| Arg269 | VDW packing (binding site floor) |
| Semi- or nonconserved a.a.* | |
| Ser91 | None |
| Ala93 | VDW packing |
| Tyr105 | Hb between Tyr OH and Siaα6 ⁻OOC— and Galβ4 O4 |
| Arg208 | Bidentate hb between Arg guanidino NH and Siaα6 O7 |

*Concerved, semi- or nonconcerved amino acids refer to a comparison between X31 Aichi and the one hundred most homologous seguences but all cited amino acids refer to X31 Aichi
**It should be noted that strains A/2/Japan/305/57 and A/PR/8/34 are not included in the one hundred most homologous sequences and that their binding of saccharides 7, 17 and 18 are significantly different from the other tested strains. Notably, they both lack the N-linked glycan at Asn165 and Trp222 bordering region B and also reveal significant differences in region C.

TABLE 2
Glycosidic torsion angles of saccharide 7 in complex with X31 Aichi

| Linkage | Angles |
|---|---|
| A | 48, 179 |
| B | 39, 170 |
| C | 73, −12 |
| D | −61, −166, 172 |
| E | −170, 21 |
| F | 55, −12 |
| G | −162, 170, 45 |
| H | 86, −154, 31 |
| I | 40, −26 |

Saccharide 7 with linkage abbreviations:
Neu5Acα2-6[G]Galβ1-4[A]GlcNAcβ1-3[F](Neu5Acα2-6[D]Galβ1-4[I]GlcNAcβ1-3[F]Galβ1-4[B]GlcNAcβ1-6[H])Galβ1-4[C]Glc

TABLE 3
Example of library of branched poly-N-acetylalctosamines
Including simple monosialylated structures.

| | |
|---|---|
| 1 | Neu5Acα2-6Galβ1-4GlcNAcβ1-3Galβ1-4Glc |
| 2 | Neu5Acα2-3Galβ1-4GlcNAcβ1-6Galβ1-4Glc |
| 3 | Neu5Acα2-6Galβ1-4GlcNAcβ1-6Galβ1-4Glc |
| 4 | Neu5Acα2-3Galβ1-4GlcNAcβ1-3Galβ1-4Glc |
| 5 | Neu5Acα2-6Galβ1-4GlcNAcβ1-3(Galβ1-4GlcNAcβ1-6)Galβ1- |
| | 4Glc |
| 6 | Neu5Acα2-3Galβ1-4GlcNAcβ1-3(Neu5Acα2-3Galβ1- |
| | 4GlcNAcβ1-3Galβ1-4GlcNAcβ1-6)Galβ1-4Glc |
| 7 | Neu5Acα2-6Galβ1-4GlcNAcβ1-3(Neu5Acα2-6Galβ1- |
| | 4GlcNAcβ1-3Galβ1-4GlcNAcβ1-6)Galβ1-4Glc |
| 8 | Neu5Acα2-6Galβ1-4GlcNAcβ1-3(Neu5Acα2-6Galβ1- |
| | 4GlcNAcβ1-6)Galβ1-4Glc |
| 9 | Neu5Acα2-3Galβ1-4GlcNAcβ1-3(Neu5Acα2-3Galβ1- |
| | 4GlcNAcβ1-6)Galβ1-4Glc |
| 10 | Neu5Acα2-6Galβ1-4GlcNAcβ1-3Galβ1-4[Fucα1-3]GlcNAcβ1- |
| | 3Galβ1-4Glc |
| 11 | Neu5Acα2-6Galβ1-4GlcNAcβ1-3Galβ1-4GlcNAcβ1-3Galβ1- |
| | 4Glc |
| 12 | Neu5Acα2-3[Galβ1-4GlcNAcβ1-3(Galβ1-4GlcNAcβ1- |
| | 6)Galβ1-4Glc] |
| 13 | Neu5Acα2-6Galβ1-4GlcNAcβ1-3(Galβ1-4[Fucα1- |
| | 3]GlcNAcβ1-6)Galβ1-4Glc |
| 14 | Neu5Acα2-6Galβ1-4GlcNAcβ1-3(Galβ1-4GlcNAcβ1-6)LNβ1- |
| | 3Galβ1-4Glc |
| 15 | Neu5Acα2-6[Galβ1-4GlcNAcβ1-3(Galβ1-4GlcNAcβ1-3Galβ1- |
| | 4GlcNAcβ1-6)Galβ1-4Glc] |
| 16 | Neu5Acα2-6Galβ1-4GlcNAcβ1-3Galβ1-4GlcNAcβ1-3 |
| | (Neu5Acα2-6Galβ1-4GlcNAcβ1-6)Galβ1-4Glc |
| 17 | Neu5Acα2-6Galβ1-4GlcNAcβ1-3(Neu5Acα2-3Galβ1- |
| | 4GlcNAcβ1-3Galβ1-4GlcNAcβ1-6)Galβ1-4Glc |
| 18 | Neu5Acα2-3Galβ1-4GlcNAcβ1-3Galβ1-4GlcNAcβ1- |
| | 3(Neu5Acα2-6Galβ1-4GlcNAcβ1-6)Galβ1-4Glc |
| 19 | Neu5Acα2-6Galβ1-4GlcNAcβ1-3Galβ1-4GlcNAcβ1- |
| | 3(Neu5Acα2-3Galβ1-4GlcNAcβ1-6)Galβ1-4Glc |
| 20 | Neu5Acα2-3Galβ1-4GlcNAcβ1-3 Galβ1-4GlcNAcβ1- |
| | 3(Neu5Acα2-3Galβ1-4GlcNAcβ1-3Galβ1-4GlcNAcβ1- |
| | 6)Galβ1-4Glc |
| 21 | Neu5Acα2-6Galβ1-4GlcNAcβ1-3Galβ1-4GlcNAcβ1- |
| | 3(Neu5Acα2-6Galβ1-4GlcNAcβ1-3Galβ1-4GlcNAcβ1- |
| | 6)Galβ1-4Glc |
| 22 | Neu5Acα2-3Galβ1-4GlcNAcβ1-3(Neu5Acα2-6Galβ1- |
| | 4GlcNAcβ1-3Galβ1-4GlcNAcβ1-6)Galβ1-4Glc |
| 23 | Neu5Acα2-6Galβ1-4GlcNAcβ1-3(Galα1-3Galβ1-4GlcNAcβ1- |
| | 3Galβ1-4GlcNAcβ1-6)Galβ1-4Glc |
| 24 | Neu5Acα2-6Galβ1-4GlcNAcβ1-3(GlcNAcβ1-3Galβ1- |
| | 4GlcNAcβ1-3Galβ1-4GlcNAcβ1-6)Galβ1-4Glc |
| 25 | [Neu5Acα2-6Galβ1-4GlcNAcβ1-3Galβ1-4Glc]₂-DADA-oxime |
| 26 | [Neu5Acα2-3Galβ1-4Glc]₂-DADA-oxime |
| 27 | [Neu5Acα2-6Galβ1-4GlcNAc]₂-DADA-oxime |
| 28 | Neu5Acα2-6Galβ1-4GlcNAcβ1-3Galβ1-4Glc-(Neu5Acα2- |
| | 6Galβ1-4GlcNAc-)DADA-oxime |

TABLE 4
Binding of different human influenza virus A strains to glycolipids.

| | Complex | | | | | |
|---|---|---|---|---|---|---|
| Influenza | gangl. of | 8-C37 | 8-C42 | 9-C37 | 9-C42 | |
| strain | human granulocytes | α6 | α6 | α3 | α3 | Globoside |
| A/Taiwan/1/86 | + | + | + | + | + | − |
| H1N1 | | | | | | |
| A/Beijing/262/95 | + | + | + | + | + | − |
| H1N1 | | | | | | |
| A/New | + | + | + | (+) | (+) | − |
| Caledonia/20/99 | | | | | | |
| A/Kiev/301/94 | + | + | + | + | + | − |
| H3N2 | | | | | | |
| A/Shangdong/9/93 | + | + | + | + | + | − |
| H3N2 | | | | | | |
| A/PR/8/34 | + | + | + | + | + | − |
| H1N1 | | | | | | |
| A/X31, | + | + | + | − | − | − |
| A/Aichi/3/75 | | | | | | |
| H3N2 | | | | | | |
| A/2/Japan/305/57 | + | − | − | + | + | − |
| H2N2 | | | | | | |
| A/Hong | + | + | + | + | + | − |
| Kong/8/68 | | | | | | |
| H3N2 | | | | | | |

C42, lysine-palmitate aminolipid;
C37-phosphatidylethanolamine.
“+” = binding;
“−” = no binding;
“(+)” = trace binding

TABLE 5
Approximate immune reactions of sera from test subjects 1-6
against synthetic peptides.

| | P1-N | P1-C Cys | P2-N | P2-mid Cys | P3-N | HA11-N Cys |
|---|---|---|---|---|---|---|
| Serum 1 | − | − | ++ | + | ++ | + |
| Serum 2 | + | + | ++++ | +++ | + | +++ |
| Serum 3 | ++ | ++ | ++ | +++ | − | ++ |
| Serum 4 | ++ | ++ | +++ | + | − | ++ |
| Serum 5 | − | − | +++ | + | + | + |
| Serum 6 | − | +++ | +++ | +++ | + | ++ |

P1, P2, And P3 indicates peptides 1-3,
HA11 is commercial peptide
N is N-terminal Biotin immobilized conjugate,
Cys-indicates Cys-conjugate,
C is C-terminal.

TABLE 6

Conserved/antigenic peptide epitopes including Peptides 1-3 in selected H1-hemagglutinins.
Prev indicates previous aminoacid belonging as additional residues in the epitope, pos
indicates the position of the aminoacid residue in the hemagglutinin sequence, Past indicates
foolowing amino acid residue belonging as additional residues in the epitope. Sequence
indicates not so frequent variants of the sequence. PrePept and Post Pept sequences are
additional conserved and/or antigenic sequences in the peptide. In column 2, s indicates
presence of possible signal peptide affecting the numbering, s13 is putative signal peptide
of 13 amino acid residue, when sequence positions are compared the signal peptides may be
deducted from the aminoacid position numbers.

| | | | | Peptide1 | | | | | | | |
|---|---|---|---|---|---|---|---|---|---|---|---|
| | | | | NSENGTC(a) | | | | PostPept1 | | | |
| | | PrePept1 | | NPENGTC(b) | | | | YPGYFADYE(a) | | | |
| Hemagglutinin type H1 | | SWSYI(a) | | NSENGIC(c) | | | | YPGYFADYE(b) | | | |
| Virus name | | prev.Pos.past | a) | prev.Pos. | a) | b | c) | c)sequence | a) | b) | |
| A/South Carolina/1/18 | H1N1 | AS79-83VE | 1 | TS88-94 | 1 | | | | 1 | | 95-103 |
| A/Finland/158/91 | H1N1,s13 | KE92-96AE | 1 | TP101-107 | | 1 | | | | 1 | 108-116 |
| A/Mongolia/111/91 | H1N1,s | VR88-92VE | 1 | TP97-103 | | | 1 | | 1 | | 104-112 |
| A/Czechoslovakia/2/88 | H1N1,s13? | KK92-96AE | 1 | TP101-107 | 1 | | | | | 1 | 108-116 |
| A/Fiji/2/88 | H1N1,s13 | KK92-96AE | 1 | TP101-107 | 1 | | | | | 1 | 108-116 |
| A/Trinidad/2/86 | H1N1,s13 | KK92-96AE | 1 | TP101-107 | 1 | | | | | 1 | 108-116 |
| A/duck/WI/259/80 | H1N1,s13 | AN92-96IE | 1 | TS101-107 | 1 | | | YPGEFIDYE | | | 108-116 |
| A/Mongolia/231/85 | H1N1,s13 | KK92-96AE | 1 | TP101-107 | 1 | | | | | 1 | 108-116 |
| A/Texas/22/90 | H1N1,s13 | KE92-96AE | 1 | TP101-107 | | 1 | | | | 1 | 108-116 |

| | | | | | | | Peptide2b | | | |
|---|---|---|---|---|---|---|---|---|---|---|
| | | | | Peptide2 | | | SYAGAS(a) | | | |
| | PrePep2 | | | GVTAAC(a) | | | SHNGKS(b) | | | |
| Hemagglutinin type H1 | SSWPNH(a) | | | GVTASC(b) | | | SHEGKS(c) | | | |
| Virus name | b)sequen. | a) | Prev.Pos.Past | Prev.Pos | a) | b) | d)sequence | a) | b) | c) |
| A/South Carolina/1/18 | | 1 | KT125-130HE | TK135-140 | 1 | | | 1 | | |
| A/Finland/158/91 | | 1 | KE138-143TV | TK148-153 | | 1 | | | 1 | |
| A/Mongolia/111/91 | | 1 | KE134-199N | TN143-148 | 1 | | | | | 1 |
| A/Czechoslovakia/2/88 | | 1 | KE138-143TV | TK148-153 | 1 | | SHKGRS | | | |
| A/Fiji/2/88 | | 1 | KE138-143TV | TK148-153 | | 1 | SHKGKS | | | |
| A/Trinidad/2/86 | | 1 | KE138-143TV | TK148-153 | | 1 | SHKGKC | | | |
| A/duck/WI/259/80 | | 1 | KA138-143ET | TK148-153 | 1 | | SYSGAS | | | |
| A/Mongolia/231/85 | RSWPKH | | KE138-143NV | TR148-153 | | 1 | SHKGKS | | | |
| A/Texas/22/90 | | 1 | KE138-143TV | TK148-153 | | 1 | | | 1 | |

TABLE 7

Conserved/antigenic peptide epitopes including Peptides 1-3 in
selected H1-hemagglutinins. The abbreviation are as in Table 6.

| | | | | | Peptide4 | | | | |
|---|---|---|---|---|---|---|---|---|---|
| | | PrePept4 | | | TDQQSLYQ(a) | | | | |
| | | WGVHHP(a) | | | GDQRAIYH(b) | | | | |
| Hemagglutinin type H1 | | WGIHHP(b) | | | KEQQNLYQ(c) | | | | |
| Virus name | | Prev.Pos.Past | a) | b) | d)sequence | a) | b) | c) | Pre.Pos.Past |
| A/South Carolina/1/18 | H1N1 | VL181-186 PT | 1 | | | 1 | | | G190-197NADAYVSVG |
| A/Finland/158/91 | H1N1,s13 | VL194-199 SN | 1 | | | | 1 | | I203-210TENAYVSVV |
| A/Mongolia/111/91 | H1N1,s | VL189-194 PN | | 1 | | | | 1 | S198-205NENAYVSVV |
| A/Czechoslovakia/2/88 | H1N1,s13? | VL194-199 SN | 1 | | | | 1 | | I203-210TENAYVSVV |
| A/Fiji/2/88 | H1N1,s13 | VL194-199 SN | 1 | | GNQRAIYH | | | | I203-210TENAYVSVV |
| A/Trinidad/2/86 | H1N1,s13 | VL194-199 SN | 1 | | | | 1 | | I203-210TENAYVSVV |
| A/duck/WI/259/80 | H1N1,s13 | VL194-199 PT | 1 | | NEQQSLYQ | | | | V203-210NADAYVSVG |
| A/Mongolia/231/85 | H1N1,s13 | VL194-199 SN | 1 | | EDQKTIYR | | | | I203-210KENAYVSVV |
| A/Texas/22/90 | H1N1,s13 | VL194-199 SN | 1 | | RDQRAIYH | | | | I203-210TENAYVSVV |

| | | | | | | PostPeptide3 | | | |
|---|---|---|---|---|---|---|---|---|---|
| | PrePept3/ | | Peptide3 | | | NYYWTLL(a) | | | |
| | PostPept4 | | RPKVRDQ(a) | | | NYYWTML(b) | | | |
| Hemagglutinin type H1 | RRFTPEI | | RPKVRGQ(b) | | | NYHWTLL(c) | | | |
| Virus name | Prev.PosPast | a) | Prev.PosPast | a) | b) | Pre.PosPast | a) | b) | c) |
| A/South Carolina/1/18 | KYN212-218 | 1 | AA221-227A | 1 | | GRM232-238EPGDTI | 1 | | |
| A/Finland/158/91 | HYS225-231 | 1 | AK234-240E | 1 | | GRI245-251EPGDTI | 1 | | |
| A/Mongolia/111/91 | NYN220-226 | 1 | AE229-235A | 1 | | GRM240-246KPGDTI | 1 | | |
| A/Czechoslovakia/2/88 | HYN225-231 | 1 | AK234-240E | 1 | | GRI245-251EPGDTI | 1 | | |
| A/Fiji/2/88 | HYN225-231 | 1 | AK234-240E | | 1 | GRI245-251EPGDTI | | 1 | |
| A/Trinidad/2/86 | HYN225-231 | 1 | AK234-240E | 1 | | GRI245-251EPGDTI | 1 | | |
| A/duck/WI/259/80 | KYN225-231 | 1 | AA234-240A | | 1 | GRM245-251DQGDTI | | | 1 |
| A/Mongolia/231/85 | NYN225-231 | 1 | AE234-240G | | 1 | GRI245-251EPGDTI | 1 | | |
| A/Texas/22/90 | HYS225-231 | 1 | AK234-240E | 1 | | GRI245-251EPGDTI | 1 | | |

TABLE 8

Conserved/antigenic peptide epitopes, Peptides 1-3, in selected H2-hemagglutinins.
The abbreviation are as in Table 6.

| | | | | | | | | | Peptide3 | | |
|---|---|---|---|---|---|---|---|---|---|---|---|
| | | Peptide1 | | | | Peptide2 | | | RPEVNGQ(a) | | |
| | | NPRNGLC(a) | | | | SQGCAV(a) | | | RPKVNGL(b) | | |
| | | NPRYSLC(b) | | | | SWACAV(b) | | | (c) | | |
| | | (c) | | | | (c) | | | (d) | | |
| Virus name | H2 | Pre.PosPast | a) | b | | Prev.PosPast | a) | b) | Prev.PosPast | a) | b) |
| A/chick./Potsdam/4705 | H2N2,s | KE99-105 | 1 | | KE99-105 | TTGG146-15 | 1 | | AT230-236GG | 1 | |
| A/Korea/426/68 | H2N2,s | KE99-105 | | 1 | KE99-105 | TTGG146-151 | SC | 1 | AA230-236GR | | 1 |

TABLE 9
Conserved/antigenic peptide epitopes including Peptides 1-3 in selected H3-hemagglutinins.

| | | Peptide1 | | | | | | | | Peptide2 |
|---|---|---|---|---|---|---|---|---|---|---|
| | | SKAFSNC(a) | | | | | PostPept1 | | | SNACKR(a) |
| | | SKAYSNC(b) | | | | | YPYDVPDY(a) | | | SYACKR(b) |
| Hemagglutinin type H3 | | STAYSNC(c) | | | | | YPYDVPDYV(b) | | | SSACKR(c) |
| Virus name | | d)sequence. | a) | b | c) | Prev.Pos. | Pos. | a) | b) | Prev.PosPast |
| A/X31 | H3N2 | | 1 | | | ER91-97 | 98-106 | 1 | | ONGG136-141GPGS |
| A/Finland/445/96 | H3N2 | | | | 1 | 91-97 | 98-106 | 1 | | 136-141 |
| A/Finland/539/97 | H3N2 | STAYSNC | | | | 91-97 | 98-106 | 1 | | 136-141 |
| A/Finland/447/96 | H3N2 | SKAYSDC | | | | 91-97 | 98-106 | 1 | | 136-141 |
| A/Finland/313/03 | H3N2 | SKADSNC | | | | 91-97 | 98-106 | 1 | | 136-141 |
| A/Finland/594/98 | H3N2 | | | 1 | | 91-97 | 98-106 | 1 | | 136-141 |
| A/Finland/587/98 | H3N2 | | | 1 | | 91-97 | 98-106 | 1 | | 136-141 |
| A/Finland/590/98 | H3N2 | | | 1 | | 91-97 | 98-106 | 1 | | 136-141 |
| A/Finland/528/97 | H3N2 | | | 1 | | 91-97 | 98-106 | 1 | | 136-141 |
| A/Finland/339/95 | H3N2 | | | 1 | | 91-97 | 98-106 | 1 | | 136-141 |
| A/Finland/380/95 | H3N2 | | | 1 | | 91-97 | 98-106 | 1 | | 136-141 |
| A/Finland/364/95 | H3N2 | | | 1 | | 91-97 | 98-106 | 1 | | 136-141 |
| A/Finland/296/93 | H3N2 | | | 1 | | 91-97 | 98-106 | 1 | | 136-141 |
| A/Finland/256/93 | H3N2 | | | 1 | | 91-97 | 98-106 | 1 | | 136-141 |
| A/Finland/321/93 | H3N2 | | | 1 | | 91-97 | 98-106 | 1 | | 136-141 |
| A/Finland/263/93 | H3N2 | | | 1 | | 91-97 | 98-106 | 1 | | 136-141 |
| A/Finland/190/92 | H3N2 | | | 1 | | 91-97 | 98-106 | 1 | | 136-141 |
| A/Finland/218/92 | H3N2 | | | 1 | | 91-97 | 98-106 | 1 | | 136-141 |
| A/Finland/191/92 | H3N2 | | | 1 | | 91-97 | 98-106 | 1 | | 136-141 |
| A/Finland/110/89 | H3N2 | | | 1 | | 91-97 | 98-106 | 1 | | 136-141 |
| A/Finland/220/92 | H3N2,s16 | | | 1 | | 107-113 | 114-122 | 1 | | 152-167 |
| A/Finland/218/92 | H3N2,s16 | | | 1 | | 107-113 | 114-122 | 1 | | 152-167 |
| A/Beijing/353/89 | H3N2 | | | 1 | | 91-97 | 98-106 | 1 | | 136-141 |
| A/Europe/C2-5/02 | H3N2 | | | 1 | | 91-97 | 98-106 | | 1 | 136-141 |
| A/Finland/C2-10/02 | H3N2 | | | 1 | | 91-97 | 98-106 | | 1 | 136-141 |
| A/Finland/12/02 | H3N2 | | | 1 | | 91-97 | 98-106 | | 1 | 136-141 |
| A/Finland/C2-17/02 | H3N2 | | | 1 | | 91-97 | 98-106 | | 1 | 136-141 |
| A/Finland/C2-14/02 | H3N2 | | | 1 | | 91-97 | 98-106 | | 1 | 136-141 |
| A/Finland/C2-13/02 | H3N2 | | | 1 | | 91-97 | 98-106 | | 1 | 136-141 |
| A/Finland/C2-7/02 | H3N2 | | | 1 | | 91-97 | 98-106 | | 1 | 136-141 |
| A/Finland/684/99 | H3N2 | | | 1 | | 91-97 | 98-106 | 1 | | 136-141 |
| A/Finland/663/99 | H3N2 | | | 1 | | 91-97 | 98-106 | 1 | | 136-141 |
| A/Finland/645/99 | H3N2 | | | 1 | | 91-97 | 98-106 | 1 | | 136-141 |
| A/Finland/455/04 | H3N2 | | | 1 | | 91-97 | 98-106 | 1 | | 136-141 |
| A/Finland/481/04 | H3N2 | | | 1 | | 91-97 | 98-106 | 1 | | 136-141 |
| A/Finland/482/04 | H3N2 | | | 1 | | 91-97 | 98-106 | 1 | | 136-141 |
| A/Finland/485/04 | H3N2 | | | 1 | | 91-97 | 98-106 | 1 | | 136-141 |
| A/Finland/486/04 | H3N2 | | | 1 | | 91-97 | 98-106 | 1 | | 136-141 |
| A/Finland/C4-22/03 | H3N2 | | | 1 | | 91-97 | 98-106 | 1 | | 136-141 |
| A/Finland/C4-23/03 | H3N2 | | | 1 | | 91-97 | 98-106 | 1 | | 136-141 |
| A/Finland/435/03 | H3N2 | | | 1 | | 91-97 | 98-106 | 1 | | 136-141 |
| A/Finland/272/03 | H3N2 | | | 1 | | 91-97 | 98-106 | 1 | | 136-141 |
| A/Finland/358/03 | H3N2 | | | 1 | | 91-97 | 98-106 | 1 | | 136-141 |
| A/Finland/437/03 | H3N2 | | | 1 | | 91-97 | 98-106 | 1 | | 136-141 |
| A/Finland/402/03 | H3N2 | | | 1 | | 91-97 | 98-106 | 1 | | 136-141 |
| A/Finland/1/02 | H3N2 | | | 1 | | 91-97 | 98-106 | 1 | | 136-141 |

| | | | | Post Pept2 | | | PrePept4 | | | | |
|---|---|---|---|---|---|---|---|---|---|---|---|
| | | | | GFFSRL(a) | | | WGVHHP(a) | | | | |
| Hemagglutinin typeH3 | | | | SFFSRL(b) | | | WGIHHP(b) | | | | |
| Virus name | a) | b) | c) | Pos,Past | a) | b) | d)sequence | a) | b) | c) | Prev.Pos.Past |
| A/X31 | 1 | | | 146-151NWLTK | 1 | | | | 1 | | YI180-185ST |
| A/Finland/445/96 | | 1 | | 146-151NWL | | 1 | | 1 | | | YI180-185 |
| A/Finland/539/97 | | 1 | | 146-151NWL | | 1 | | 1 | | | YI180-185 |
| A/Finland/447/96 | | 1 | | 146-151NWL | | 1 | | 1 | | | YI180-185 |
| A/Finland/313/03 | | | 1 | 146-151NWL | | 1 | | 1 | | | YI180-185 |
| A/Finland/594/98 | | 1 | | 146-151NWL | | 1 | | 1 | | | YI180-185 |
| A/Finland/587/98 | | 1 | | 146-151NWL | | 1 | | 1 | | | YI180-185 |
| A/Finland/590/98 | | 1 | | 146-151NWL | | 1 | | 1 | | | YI180-185 |
| A/Finland/528/97 | | 1 | | 146-151NWL | | 1 | | 1 | | | YI180-185 |
| A/Finland/339/95 | | 1 | | 146-151NWL | | 1 | | 1 | | | YI180-185 |
| A/Finland/380/95 | | 1 | | 146-151NWL | | 1 | | 1 | | | YI180-185 |
| A/Finland/364/95 | | 1 | | 146-151NWL | | 1 | | 1 | | | YI180-185 |
| A/Finland/296/93 | | 1 | | 146-151NWL | | 1 | | 1 | | | YI180-185 |
| A/Finland/256/93 | | 1 | | 146-151NWL | | 1 | | 1 | | | YI180-185 |
| A/Finland/321/93 | | 1 | | 146-151NWL | | 1 | | 1 | | | YI180-185 |
| A/Finland/263/93 | | 1 | | 146-151NWL | | 1 | | 1 | | | YI180-185 |
| A/Finland/190/92 | | 1 | | 146-151NWL | | 1 | | 1 | | | YI180-185 |
| A/Finland/218/92 | | 1 | | 146-151NWL | | 1 | | 1 | | | YI180-185 |
| A/Finland/191/92 | | 1 | | 146-151NWL | | 1 | | 1 | | | YI180-185 |
| A/Finland/110/89 | | 1 | | 146-151NWL | | 1 | | 1 | | | YI180-185 |
| A/Finland/220/92 | | 1 | | 146-151NWL | | 1 | | 1 | | | YI180-185 |
| A/Finland/218/92 | | 1 | | 146-151NWL | | 1 | | 1 | | | YI180-185 |
| A/Beijing/353/89 | | 1 | | 146-151NWL | | 1 | | 1 | | | YI180-185 |
| A/Europe/C2-5/02 | | | 1 | 146-151NWL | | 1 | | 1 | | | YI180-185 |
| A/Finland/C2-10/02 | | | 1 | 146-151NWL | | 1 | | 1 | | | YI180-185 |
| A/Finland/12/02 | | | 1 | 146-151NWL | | 1 | WVGLHP | | | | YI180-185 |
| A/Finland/C2-17/02 | | | 1 | 146-151NWL | | 1 | | 1 | | | YI180-185 |
| A/Finland/C2-14/02 | | | 1 | 146-151NWL | | 1 | | 1 | | | YI180-185 |
| A/Finland/C2-13/02 | | | 1 | 146-151NWL | | 1 | | 1 | | | YI180-185 |
| A/Finland/C2-7/02 | | | 1 | 146-151NWL | | 1 | | 1 | | | YI180-185 |
| A/Finland/684/99 | | | 1 | 146-151NWL | | 1 | | 1 | | | YI180-185 |
| A/Finland/663/99 | | | 1 | 146-151NWL | | 1 | | 1 | | | YI180-185 |
| A/Finland/645/99 | | | 1 | 146-151NWL | | 1 | | 1 | | | YI180-185 |
| A/Finland/455/04 | | | 1 | 146-151NWL | | 1 | | 1 | | | YI180-185 |
| A/Finland/481/04 | | | 1 | 146-151NWL | | 1 | | 1 | | | YI180-185 |
| A/Finland/482/04 | | | 1 | 146-151NWL | | 1 | | 1 | | | YI180-185 |
| A/Finland/485/04 | | | 1 | 146-151NWL | | 1 | | 1 | | | YI180-185 |
| A/Finland/486/04 | | | 1 | 146-151NWL | | 1 | | 1 | | | YI180-185 |
| A/Finland/C4-22/03 | | | 1 | 146-151NWL | | 1 | | 1 | | | YI180-185 |
| A/Finland/C4-23/03 | | | 1 | 146-151NWL | | 1 | | 1 | | | YI180-185 |
| A/Finland/435/03 | | | 1 | 146-151NWL | | 1 | | 1 | | | YI180-185 |
| A/Finland/272/03 | | | 1 | 146-151NWL | | 1 | | 1 | | | YI180-185 |
| A/Finland/358/03 | | | 1 | 146-151NWL | | 1 | | 1 | | | YI180-185 |
| A/Finland/437/03 | | | 1 | 146-151NWL | | 1 | | 1 | | | YI180-185 |
| A/Finland/402/03 | | | 1 | 146-151NWL | | 1 | | 1 | | | YI180-185 |
| A/Finland/1/02 | | | 1 | 146-151NWL | | 1 | | 1 | | | YI180-185 |

### REFERENCES

Glick G D, et al, (1991) J. of Biological Chemistry 266(35):23660-23669
Hennecke J, et al, (2000) The EMBO Journal 19(21):5611-5624
Lin A H & Cannon P M (2002) Virus Res. 83(1-2):43-56
Lu Y, et al (2002) Int Arch Allergy Immunol. 127(3):245-250
Sauter N K, et al (1992) Proc. Natl. Acad. Sci. USA 89:324-328
Suzuki Y, et al (1992) Virology 189:121-131

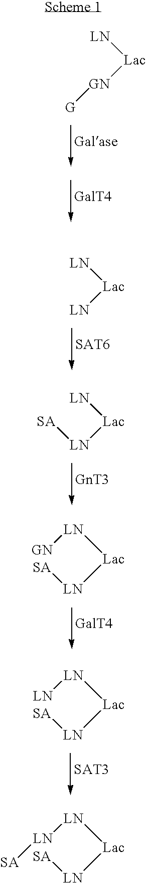

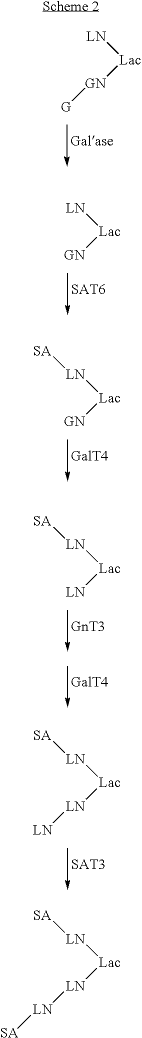

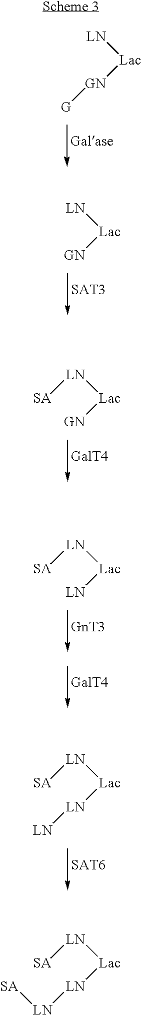

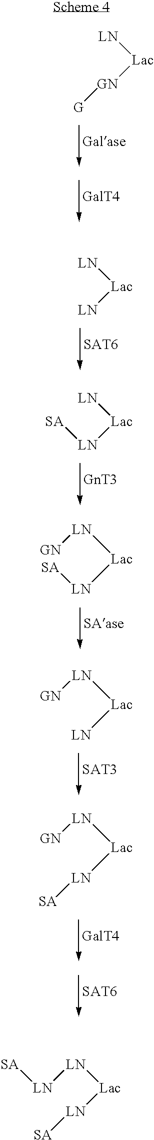

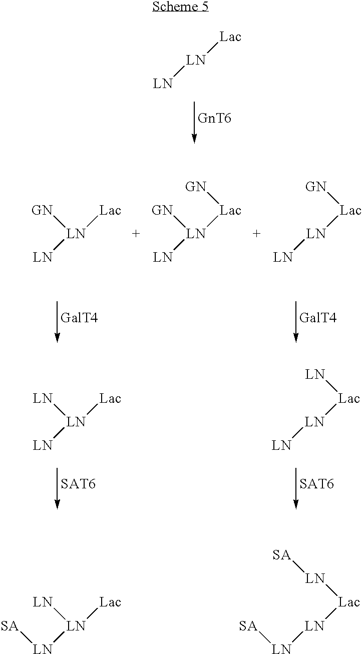

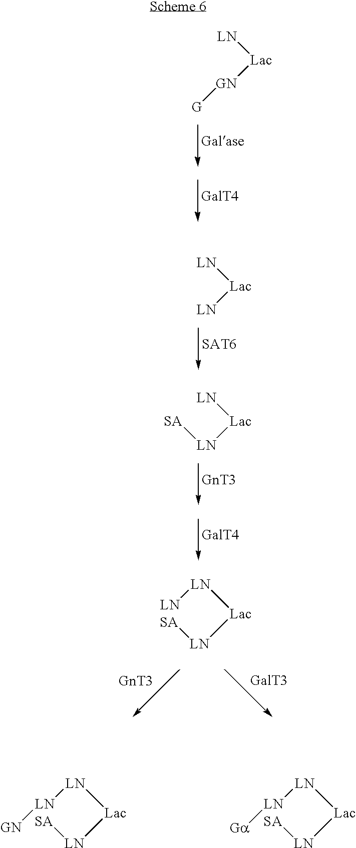

1-68. (canceled)

69. A method for evaluating the potential of a chemical entity to bind to a peptide epitope derived from the divalent sialoside binding site of hemagglutinin protein of influenza virus comprising the steps of:
(i) contacting said chemical entity with said peptide under conditions that allow said chemical entity to bind said peptide; and
(ii) detecting the presence of a complex of said chemical entity and said peptide.

70. The method according to claim 69 wherein said binding site is defined by structure coordinates of influenza hemagglutinin amino acids Tyr98, Gly135, Trp153, His183, Leu194 and Gly225 of Region A; and Ser95, Val223, Arg224, Gly225 and Asn165 of Region B; and Thr65, Ser71, Glu72, Ser95, Gly98, Pro99, Tyr100 and Arg269 of Region C according to FIG. 1.

71. The method according to claim 70, wherein said large binding site is further defined by at least one of the structure coordinates of influenza hemagglutinin semi- or nonconserved amino acids Gly134, Asn137, Ala138, Thr155, Glu190 and Leu226 of Region A; Phe94, Asn96, Asn137, Ala138, Lys140 and Arg207 of Region B; Ser91, Ala 93, Tyr105 and Arg208 of Region C.

72. The method according to claim 69, wherein said peptide is selected from the group consisting of KVR-region peptides of hemagglutinin type 1, WVR-region peptides of hemagglutinin type 3, KVN-region peptides of hemagglutinin type 5, TSNSENGT(C)-region of hemagglutinin type 1, SKAFSN(C)-region peptides of hemagglutinin type 3, KXNPVNXL(C) region of hemagglutinin type 5, TTKGVTAA(C)-region of hemagglutinin type 1, GGSNA-region peptides of hemagglutinin type 3, and DASSGVSSA(C)PY-region of hemagglutinin type 5.

73. The method according to claim 69, wherein said chemical entity is an antibody.

74. The method according to claim 69, wherein the method is used for vaccine development.

75. The method according to claim 69, wherein the method is used for screening binding agents from a library.

76. The method according to claim 69, wherein the method is used for screening antibodies from human serum.

77. The method according to claim 69, wherein said peptide epitope is a peptide set forth in SEQ ID NO:12.

78. A method for producing a peptide vaccine against influenza comprising steps of:
preparing a peptide conjugate comprising at least two peptides selected from the group consisting of KVR-region peptides of hemagglutinin type 1, WVR-region peptides of hemagglutinin type 3, KVN-region peptides of hemagglutinin type 5, TSNSENGT(C)-region of hemagglutinin type 1, SKAFSN(C)-region peptides of hemagglutinin type 3, KXNPVNXL(C)-region of hemagglutinin type 5, TTKGVTAA(C)-region of hemagglutinin type 1, GGSNA-region peptides of hemagglutinin type 3, and DASSGVSSA(C)PY-region of hemagglutinin type 5;
administering said peptide conjugate to an animal; and
monitoring the animal in order to detect immune response against the peptide conjugate.

79. A peptide conjugate comprising at least two peptides selected from the group consisting of KVR-region peptides of hemagglutinin type 1, WVR-region peptides of hemagglutinin type 3, KVN-region peptides of hemagglutinin type 5, TSNSENGT(C)-region of hemagglutinin type 1, SKAFSN(C)-region peptides of hemagglutinin type 3, KXNPVNXL(C)-region of hemagglutinin type 5, TTKGVTAA(C)-region of hemagglutinin type 1, GGSNA-region peptides of hemagglutinin type 3, DASSGVSSA(C)PY-region of hemagglutinin type 5 and a peptide set forth in SEQ ID NO:12.

80. The peptide conjugate according to claim 79, comprising a carrier, other immunogenic peptides, or an adjuvant, wherein said peptide is optionally covalently linked to the surface of a carrier protein.

81. A vaccine composition comprising the peptide conjugate according to claim 79.

82. A method of identifying influenza virus in a biological sample, the method comprising: (a) contacting the biological sample with a nucleic acid primers amplifying the part of virus genome encoding for the divalent sialoside binding site of hemagglutinin protein under conditions allowing polymerase chain reaction; and (b) determining the sequence of the amplified nucleic acid in the biological sample, to thereby identify the presence and type of influenza virus.

83. The method according to claim 69, for identifying influenza virus in a biological sample, the method comprising: (a) contacting the biological sample with an antibody or antibody fragment specifically recognizing the divalent sialoside binding site of hemagglutinin protein; and (b) detecting immunocomplexes including said antibody or antibody fragment in the biological sample, to thereby identify the presence and type of influenza virus in the biological sample.

84. A method for determining nucleic acid or amino acid sequence of the divalent sialoside binding site of a hemagglutinin protein of influenza virus comprising the steps of:
(a) isolating genomic nucleic acid of an influenza virus; and
(b) sequencing a nucleic acid sequence encoding the cysteine 97 region, cysteine 139 region and the region of amino acids 220-226 as defined by the amino acid sequence of X31-hemaglutinin,
wherein said method optionally comprises a further step of designing peptides for influenza vaccine development based on the sequencing results obtained in step (b).

85. A method for evaluating the potential of a chemical entity to bind to:
a) a molecule or molecular complex comprising a large binding site defined by structure coordinates of influenza hemagglutinin amino acids Tyr98, Gly135, Trp153, His183, Leu194 and Gly225 of Region A; and Ser95, Val223, Arg224, Gly225 and Asn165 of Region B; and Thr65, Ser71, Glu72, Ser95, Gly98, Pro99, Tyr100 and Arg269 of Region C according to FIG. 1; or
b) a homologue of said molecule or molecular complex, wherein said homologue comprises a binding site that has a root mean square deviation from the backbone atoms of said amino acids of not more than 1.5 Å comprising the steps of:
(i) employing computational means to perform a fitting operation between the chemical entity and the large binding site of the molecule or molecular complex; and
(ii) analyzing the results of said fitting operation to quantify the association between the chemical entity and the large binding site
and wherein said large binding site is optionally further defined by at least one of the structure coordinates of influenza hemagglutinin semi- or nonconserved amino acids Gly134, Asn137, Ala138, Thr155, Glu190 and Leu226 of Region A; Phe94, Asn96, Asn137, Ala138, Lys140 and Arg207 of Region B; Ser91, Ala 93, Tyr105 and Arg208 of Region C.

86. The method according to claim 85 for identifying a potential agonist or antagonist of a molecule comprising a large binding site of influenza hemagglutinin comprising the steps of:
a) using the atomic coordinates of influenza hemagglutinin amino acids Tyr98, Gly135, Trp153, His183, Leu194 and Gly225 of Region A; and Ser95, Val223, Arg224, Gly225 and Asn165 of Region B; and Thr65, Ser71, Glu72, Ser95, Gly98, Pro99, Tyr100 and Arg269 of Region C according to FIG. 1 +/−a root mean square deviation from the backbone atoms of said amino acids of not more than 1.5 Å, to generate a three-dimensional structure of a molecule comprising a large binding pocket of influenza hemagglutinin;
b) employing said three-dimensional structure to design or select said agonist or antagonist;
c) synthesizing said agonist or antagonist; and
d) contacting said agonist or antagonist with said molecule to determine the ability of said agonist or antagonist to interact with said molecule.

87. A computer for producing a three dimensional representation of:
a) a molecule or a molecular complex, wherein said molecule or molecular complex comprises a binding site defined by structure coordinates of influenza hemagglutinin amino acids Tyr98, Gly135, Trp153, His183, Leu194 and Gly225 of Region A; and Ser95, Val223, Arg224, Gly225 and Asn165 of Region B; and Thr65, Ser71, Glu72, Ser95, Gly98, Pro99, Tyr100 and Arg269 of Region C according to FIG. 1; or
b. a homologue of said molecule or molecular complex, wherein said homologue comprises a binding site that has a root mean square deviation from the backbone atoms of said amino acids not more than 1.5 Å, wherein said computer comprises:
c. a computer-readable data storage medium comprising a data storage material encoded with computer-readable data, wherein said data comprises the structure coordinates of influenza hemagglutinin amino acids Tyr98, Gly135, Trp153, His183, Leu194 and Gly225 of Region A; and Ser95, Val223, Arg224, Gly225 and Asn165 of Region B; and Thr65, Ser71, Glu72, Ser95, Gly98, Pro99, Tyr100 and Arg269 of Region C according to FIG. 1;
d. a working memory for storing instructions for processing said computer-readable data;
e. a central processing unit coupled to said working memory and to said computer-readable data storage medium for processing said computer-machine readable data into said three-dimensional representation; and
f. a display coupled to said central-processing unit for displaying said three-dimensional representation.

88. A divalent alpha-sialoside, wherein the distance between sialic acid residues is between about 25 Å and 65 Å and which comprises a spacer between sialyl-oligosaccharide residues with length about 8-15 Å, wherein the spacer comprises 2-4 N-acetyllactosamines and Gal residue or analogs thereof or
the spacer is a flexible comprising 8-16 atomic bonds between ring structures of the oligosaccharide sequences and sialic acid is NeuNAc or natural or synthetic sialic acid structural analogue capable of replacing Neu5Ac in one or both of the sialic acid binding sites as defined in FIG. 1
and the sialosides comprise alpha3- and/or alpha6-sialylated di-, tri-, tetra, or pentasaccharides,
and the oligosaccharides are linked from the reducing end by the spacer.

89. The sialoside according to claim 88, wherein said sialoside is for the treatment or prevention of influenza.

90. A method for identifying a modulator of binding between the large binding site of influenza hemagglutinin and its ligand divalent sialoside, comprising steps of:
(a) contacting the large binding site of influenza hemagglutinin and its ligand in the presence and in the absence of a putative modulator compound;
(b) detecting binding between the large binding site of influenza hemagglutinin and its ligand in the presence and absence of the putative modulator; and
(c) identifying a modulator compound in view of decreased or increased binding between the large binding site of influenza hemagglutinin and its ligand in the presence of the putative modulator, as compared to binding in the absence of the putative modulator, wherein the modulator binds to peptide epitope according to claim 69.

91. The method according to claim 90, further comprising a step of:
(d) making a modulator composition by formulating a modulator identified according to step (c) in a pharmaceutically acceptable carrier.

92. A method for selecting peptide epitopes for immunization and developing peptide vaccines against influenza comprising at least one di- to decapeptide epitope of the large binding site described in Table 1, wherein the method involves analysis according to the claim 69 for antibody as a chemical entity blocking the large binding site.

93. The method according to the claim 92, wherein said peptide comprises at least two conserved amino acid residues from region B in Table 1.
